(19) United States
(12) Patent Application Publication
(10) Pub. No.: US 2022/0221443 A1
(43) Pub. Date: Jul. 14, 2022

(54) BIOMARKER DETECTION FOR CANCER DIAGNOSIS AND PROGNOSIS

(71) Applicant: IP2IPO Innovations Limited, London (GB)

(72) Inventors: George Hanna, Northwood (GB); Bhamini Vadhwana, Hounslow (GB); Ilaria Belluomo, London (GB); Piers Boshier, London (GB)

(21) Appl. No.: 17/614,374

(22) PCT Filed: May 28, 2020
(86) PCT No.: PCT/GB2020/051285

§ 371 Date: Nov. 26, 2021

(30) Foreign Application Priority Data
May 29, 2019 (GB) 1907550.6

(51) Int. Cl. G01N 33/497; G01N 33/574; G01N 33/68

(52) U.S. Cl. CPC G01N 33/4972; G01N 33/574; G01N 2033/4975; G01N 33/6848; G01N 33/6812

(57) ABSTRACT

The invention relates to a method for diagnosing a subject suffering from cancer, or a pre-disposition thereto. The method comprises detecting, in a bodily sample from a test subject, the concentration of a signature compound resulting from the metabolism of at least one sugar, and/or at least one amino acid or a precursor thereof, and/or at least one polyol present in a composition previously administered to the subject. The sugar is present in the composition at a concentration of more than 20,000 mg/100 ml, the amino acid or a precursor thereof is present in the composition at a concentration of at least 500 mg/ml, and the polyol is present in the composition at a concentration of more than 25,000 mg/100 ml. The method further comprises comparing this concentration with a reference for the concentration of the signature compound in an individual who does not suffer from cancer. In particular, an increase or decrease in the concentration of the signature compound compared to the reference, suggests that the subject is suffering from cancer, or has a pre-disposition thereto, or provides a negative prognosis of the subject's condition.

The present invention relates to the detection of biomarkers, and particularly although not exclusively, to methods, compositions and kits for the detection of biological markers for diagnosing various conditions, such as cancer. In particular, the invention relates to the detection of compounds as diagnostic and prognostic markers for detecting cancer, such as oesophago-gastric cancer or metastasised cancer.

Oesophageal adenocarcinoma is among the most common five cancers and has the fastest rising incidence of any cancer in the Western population. The UK has the highest incidence of oesophageal adenocarcinoma worldwide. Stomach cancer is the third leading cause of cancer death worldwide. Five-year survival for oesophageal and gastric cancer in the UK remains very poor (13% and 18% respectively), among the worst in Europe. The key to improving cancer-survival is earlier diagnosis. However, symptoms are non-specific and commonly-shared with benign diseases. By the time symptoms become cancer-specific, the disease is often at an advanced stage with poor prognosis. Cancer burden and unnecessary investigations of patients with non-specific symptoms result in substantial costs. There is, thus, an urgent need for a non-invasive test for patients with non-specific gastrointestinal symptoms in order to effectively triage patients to have endoscopy and other diagnostic modalities.

Prior research has shown an association between oesophago-gastric cancer and volatile organic compounds (VOCs), and an approach for its diagnosis is exhaled breath testing. Researchers using gas chromatography mass spectrometry (GC-MS) have suggested the existence of a breath volatile organic compounds (VOCs) profile specific to a specific cancer [4]. GC-MS is a good technique for VOC identification, but it is semi-quantitative in nature, unless robust calibration curves employed, and therefore limited in its ability of research findings to be reproduced by different research groups. Furthermore, there is a substantial analytical time for each sample, which does not naturally lend itself to high throughput analysis. Direct injection mass spectrometry, such as selected ion flow tube mass spectrometry (SIFT-MS) and proton transfer reaction time of flight mass spectrometry (PTR-ToF-MS) have the advantage of being quantitative and permit real-time analysis [5,6].

What is required is a reliable non-invasive diagnostic test to identify patients suffering from cancers, such as oesophago-gastric cancer. A diagnostic method to identify those patients with cancer would be of immense benefit to patients and would raise the possibility of early treatment and improved prognosis.

The inventors had previously developed a non-invasive test for cancer based on the detection of signature compounds, such as volatile organic compounds (VOCs), in exhaled breath. The inventors have now developed new methods and compositions that result in improved accuracy and more rapid testing, which is achieved by means of administering optimised concentrations of an oral stimulus foodstuff (e.g. a drink, capsule or solid foodstuff), which transiently induces or “stimulates” cancer to produce greater quantities of distinctive signature compounds (e.g. VOCs), and thereby improving test performance and diagnostic and/or prognostic accuracy. This will allow patients with non-specific symptoms, yet at a high-risk of oesophago-gastric cancer, to be identified earlier and referred for further investigation and treatment.

Accordingly, in a first aspect of the invention, there is provided a method for diagnosing a subject suffering from cancer, or a pre-disposition thereto, or for providing a prognosis of the subject's condition, the method comprising:
(i) detecting, in a bodily sample from a test subject, the concentration of a signature compound resulting from the metabolism of at least one sugar and/or at least one amino acid or a precursor thereof and/or at least one polyol present in a composition previously administered to the subject, wherein the sugar is present in the composition at a concentration of more than 20,000 mg/100 ml, the amino acid or a precursor thereof is present in the composition at a concentration of at least 500 mg/ml and the polyol is present in the composition at a concentration of more than 25,000 mg/100 ml; and
(ii) comparing this concentration with a reference for the concentration of the signature compound in an individual who does not suffer from cancer,

wherein an increase or a decrease in the concentration of the signature compound compared to the reference, suggests that the subject is suffering from cancer, or has a pre-disposition thereto, or provides a negative prognosis of the subject's condition.

Detection step (i) may comprise detecting a signature compound up to 30 minutes, up to 25 minutes, up to 20 minutes, up to 15 minutes, up to 10 minutes or up to 5 minutes from administration of the composition comprising at least one sugar and/or an amino acid or a precursor thereof and/or at least one polyol. Detection step (i) may comprise detecting a signature compound in less than 30 minutes, in less than 25 minutes, in less than 20 minutes, in less than 15 minutes, in less than 10 minutes or in less than 5 minutes from administration of the composition comprising at least one sugar and/or an amino acid or a precursor thereof and/or at least one polyol. Preferably, the detection step is performed when the composition comprises at least one sugar which is previously administered to the test subject.

Detection step (i) may further comprise detecting a signature compound at between 30 and 60 minutes from administration of the composition comprising at least one sugar and/or an amino acid or a precursor thereof and/or at least one polyol, more preferably between 30 and 55 minutes, or between 30 and 50 minutes, or between 30 and 45 minutes, or between 30 and 40 minutes, or between 35 and 60 minutes, or between 35 and 55 minutes, or between 35 and 50 minutes, or between 35 and 45 minutes, or between 35 and 40 minutes from administration of the composition comprising at least one sugar and/or an amino acid or a precursor thereof and/or at least one polyol. Preferably, detection step (i) further comprises detecting a second signature compound at between 35 and 45 minutes from administration of the composition comprising at least one sugar and/or an amino acid or a precursor thereof and/or at least one polyol. Preferably, such detection step is performed when the composition comprises at least one amino acid and/or at least one polyol.

Thus, preferably, detection step (i) comprises:
a) detecting a signature compound up to 30 minutes, up to 25 minutes, up to 20 minutes, up to 15 minutes, up to 10 minutes or up to 5 minutes, in less than 30 minutes, in less than 25 minutes, in less than 20 minutes, in less than 15 minutes, in less than 10 minutes or in less than 5 minutes from administration of the composition comprising at least one sugar and/or an amino acid or a precursor thereof and/or at least one polyol; and
b) detecting a signature compound between 30 and 60 minutes from administration of the composition comprising at least one sugar and/or an amino acid or a precursor thereof and/or at least one polyol, more preferably between 30 and 55 minutes, or between 30 and 50 minutes, or between 30 and 45 minutes, or between 30 and 40 minutes, or between 35 and 60 minutes, or between 35 and 55 minutes, or between 35 and 50 minutes, or between 35 and 45 minutes, or between 35 and 40 minutes from administration of the composition comprising at least one sugar and/or an amino acid or a precursor thereof and/or at least one polyol.

Preferably, an increase in the concentration of the signature compound compared to the reference, suggests that the subject is suffering from cancer, or has a pre-disposition thereto, or provides a negative prognosis of the subject's condition Preferably, the increase in the concentration of the signature compound is at least a 10%, 20%, 30%, 40%, 50%, 100%, 200%, 300%, 400%, 500%, 600%, 700%, 800%, 900% or 1000% increase in the concentration of signature compound when compared to the reference.

Preferably, the sugar is present at a concentration of at least 20,000 mg/100 ml, at least 20,500 mg/100 ml, at least 21,000 mg/100 ml, at least 25,000 mg/100 ml, at least 50,000 mg/100 ml or at least 75,000 mg/100 ml. Preferably, the sugar is present at a concentration of about 25,000 mg/100 ml. Preferably, the sugar is present at a concentration of more than 20,000 mg/100 ml, more than 20,500 mg/100 ml, more than 21,000 mg/100 ml, more than 25,000 mg/100 ml, more than 50,000 mg/100 ml or more than 75,000 mg/100 ml.

Preferably, the composition comprises sugar, preferably at a concentration of between about 20,000 mg/100 mL and 10,0000 mg/100 mL, more preferably between 25,000 mg/100 mL and 75,000 mg/100 mL.

The skilled person would understand that the term sugar may refer to mono, di, tri, oligo and poly-saccharides or sugar alcohols. The sugar may be selected from a group consisting of: D-glucose, D-sucrose, D-lactose, D-fructose, D-mannose, D-gulose, D-galactose, D-Xylose, D-arabinose, D-lyxose, D-ribose, D-allose, D-altrose, D-talose, D-idose, L-arabinose, L-rhamnose, L-xylulose, di-, trioligo and poly-saccharides, sorbitol, c4, c7 and >c8 monosaccharides, sorbitol, mannitol, maltitol, lactitol, erythritol.

Preferably, the sugar is glucose, sorbitol, mannose or lactose. More preferably, the sugar is glucose, mannose or lactose. Most preferably, the sugar is glucose or lactose.

Thus, preferably, the composition comprises glucose, and preferably glucose is present in the composition at a concentration of at least 25,000 mg/100 ml. More preferably, the sugar is glucose and is present in the composition at a concentration of at least 25,000 mg/100 ml and the signature compound is detected up to 10 minutes from administration of the composition comprising glucose.

The composition administered to the subject may comprise citric acid. This may be instead of, or in addition to, the sugar. Preferably, citric acid is used in combination with the sugar. Preferably, the sugar is glucose. Thus, preferably the composition comprises citric acid and glucose.

Preferably, the citric acid is present in the composition at a concentration of at least 1,000 mg/100 ml, at least 1,100 mg/100 ml, at least 1,200 mg/100 ml, at least 1,300 mg/100 ml or at least 1,400 mg/100 ml. Preferably, the citric acid is present at a concentration of about 1,400 mg/100 ml.

Thus, preferably, the composition comprises glucose and citric acid, and preferably glucose is present in the composition at a concentration of at least 25,000 mg/100 ml, and the citric acid is present in the composition at a concentration of at least 1,400 mg/100 ml. More preferably, the composition comprises glucose present in the composition at a concentration of at least 25,000 mg/100 ml, and citric acid present in the composition at a concentration of at least 1,400 mg/ml, and the signature compound is detected up to 10 minutes from administration of the composition comprising glucose and citric acid.

In another embodiment, the composition preferably comprises an amino acid, preferably at a concentration of at least 500 mg/100 ml, at least 1000 mg/100 ml, at least 2000 mg/100 ml, at least 3000 mg/100 ml, at least 4000 mg/100 ml, at least 5000 mg/100 ml, or at least 6000 mg/100 ml.

Preferably, the composition comprises an amino acid, preferably at a concentration of more than 500 mg/100 ml, more than 1000 mg/100 ml, more than 2000 mg/100 ml, more than 3000 mg/100 ml, more than 4000 mg/100 ml, more than 5000 mg/100 ml, or more than 6000 mg/100 ml.

Preferably, the amino acid is present in the composition at a concentration of between 500 mg/100 ml and 10,000 mg/100 ml, 500 mg/100 ml and 6000 mg/100 ml, between 500 mg/100 ml and 5000 mg/100 ml, between 500 mg/100 ml and 4000 mg/100 ml, between 500 mg/100 ml and 3000 mg/100 ml, between 500 mg/100 ml and 2500 mg/100 ml, between 500 mg/100 ml and 2000 mg/100 ml, between 1000 mg/100 ml and 10000 mg/100 ml, between 1500 mg/100 ml and 10000 mg/100 ml, between 2000 mg/100 ml and 10000 mg/100 ml, between 2500 mg/100 ml and 10000 mg/100 ml, between 3000 mg/100 ml and 10000 mg/100 ml, between 4000 mg/100 ml and 10000 mg/100 ml, between 5000 mg/100 ml and 10000 mg/100 ml, between 6000 mg/100 ml and 10000 mg/100 ml, between 1000 mg/100 ml and 5000 mg/100 ml, between 1000 mg/100 ml and 3000 mg/100 ml, between 1000 mg/100 ml and 2500 mg/100 ml, between 1000 mg/100 ml and 2000 mg/100 ml, between 1500 mg/100 ml and 10000 mg/100 ml, between 1500 mg/100 ml and 5000 mg/100 ml, between 1500 mg/100 ml and 3000 mg/100 ml, between 1500 mg/100 ml and 2500 mg/100 ml, or between 1500 mg/100 ml and 2000 mg/100 ml.

Preferably, the amino acid is present in the composition at a concentration of about 2000 mg/ml.

The amino acid may be selected from a group consisting of: tyrosine, glutamic acid, glutamate, phenylalanine, tryptophan, proline and histidine.

Preferably, when the amino acid is glutamic acid, the concentration of amino acid is at least 5,000 mg/100 ml, is at least 5,100 ml/100 ml, is at least 5,200 mg/100 ml, is at least 5,300 mg/100 ml, is at least 5,400 mg/100 ml, is at least 5,500 mg/100 ml, is at least 6000 mg/100 ml, more than 5,000ml/100 ml, more than 5,100 mg/100 ml, more than 5,200 mg/100 ml, more than 5,300 mg/100 ml, more than 5,400 mg/100 ml, more than 5,500 mg/100 ml, or more than 6,000 mg/100 ml. Preferably, when the amino acid is glutamic acid, the concentration of amino acid is between 1,800 mg/100 ml and 2,200 mg/100 ml, between 1,900 mg/100 ml and 2,100 mg/100 ml. Preferably, when the amino acid is glutamic acid, the concentration of amino acid is 1,900 mg/100 ml, 2,000 mg/100 ml, 2,100 mg/100 ml, 2,200 mg/100 ml or 2,300 mg/100 ml. Preferably when the amino acid is glutamic acid, the concentration of amino acid is 2,100 mg/ml. In one embodiment, however, the amino acid is not glutamic acid.

Most preferably, the amino acid is tyrosine.

Thus, preferably, the composition comprises tyrosine and preferably tyrosine is present in the composition at a concentration of at least 2,000 mg/100 ml. More preferably, the amino acid is tyrosine and is present in the composition at a concentration of at least 2,000 mg/100 ml and the signature compound is detected between 35 and 45 minutes from administration of the composition comprising tyrosine.

The composition administered to the subject may comprise an amino acid precursor. This may be instead of, or in addition to, the amino acid and/or sugar. Preferably, the amino acid precursor is phenylalanine. Preferably, the amino acid precursor is used in combination with its respective amino acid. Thus, preferably the composition comprises tyrosine and phenylalanine.

Preferably, the amino acid precursor is present in the composition at a concentration of at least 500 mg/100 ml, at least 1000 mg/100 ml, at least 2000 mg/100 ml, at least 3000 mg/100 ml at least 4000 mg/100 ml or at least 5000 mg/100 ml. Preferably, the amino acid precursor is present in the composition at a concentration of at least 500 mg/100 ml, at least 1000 mg/100 ml, at least 2000 mg/100 ml, at least 3000 mg/100 ml at least 4000 mg/100 ml or at least 5000 mg/100 ml. Preferably, the amino acid precursor is present in the composition at a concentration of between 500 mg/100 ml and 10000 mg/100 ml, between 500 mg/100 ml and 5000 mg/100 ml, is between 500 mg/100 ml and 4000 mg/100 ml, between 500 mg/100 ml and 3000 mg/100 ml, between 500 mg/100 ml and 2500 mg/100 ml, between 500 mg/100 ml and 2000 mg/100 ml, between 1000 mg/100 ml and 10000 mg/100 ml, between 1500 mg/100 ml and 10000 mg/100 ml, between 2000 mg/100 ml and 10000 mg/100 ml, between 2500 mg/100 ml and 10000 mg/100 ml, between 3000 mg/100 ml and 10000 mg/100 ml, between 1000 mg/100 ml and 5000 mg/100 ml, between 1000 mg/100 ml and 3000 mg/100 ml, between 1000 mg/100 ml and 2500 mg/100 ml, between 1000 mg/100 ml and 2000 mg/100 ml, between 1500 mg/100 ml and 10000 mg/100 ml, 1500 mg/100 ml and 5000 mg/100 ml, between 1500 mg/100 ml and 3000 mg/100 ml, between 1500 mg/100 ml and 2500 mg/100 ml, or between 1500 mg/100 ml and 2000 mg/100 ml.

Preferably, the amino acid precursor is phenylalanine. Preferably, phenylalanine is present at a concentration of 3000 mg/100 ml.

Preferably, the composition comprises phenylalanine and tyrosine.

In one embodiment, the composition comprises tyrosine, phenylalanine and glutamic acid. Preferably, tyrosine is present at a concentration of at least 2,000 mg/100 ml, phenylalanine is present at a concentration of at least 3,000 mg/100 ml, and glutamic acid is present at a concentration of at least 2,100 mg/100 ml.

Preferably, the polyol is present in the composition at a concentration of more than 25,000 mg/100 ml. Preferably, the polyol is present in the composition at a concentration of more than 26,000 mg/100 ml, more than 27,000 mg/100 ml, more than 28,000 mg/100 ml, or more than 29,000 mg/100 ml. Preferably, the polyol is present in the composition at a concentration of more than 30,000 mg/100 ml, more than 35,000 mg/100 ml, more than 40,000 mg/ml, more than 45,000 mg/100 ml, more than 50,000 mg/100 ml. Preferably, the polyol is present in the composition at a concentration of at least 30,000 mg/100 ml, at least 35,000 mg/100 ml, at least 40,000 mg/ml, at least 45,000 mg/100 ml, at least 50,000 mg/100 ml.

Preferably, the polyol is present in the composition at a concentration of 50,000 mg/100 ml. Most preferably, the polyol is present in the composition at a concentration of between 23,000 mg/100 ml and 27,000 mg/100 ml, or between 24,000 mg/100 ml and 26,000 mg/100 ml.

Preferably, the polyol is glycerol. Preferably, glycerol is present in the composition at a concentration of more than 30,000 mg/ml, more preferably 50,000 mg/100 ml. Most preferably, the glycerol is present in the composition at a concentration of between 23,000 mg/100 ml and 27,000 mg/100 ml, or between 24,000 mg/100 ml and 26,000 mg/100 ml.

In one embodiment, the at least one sugar and/or at least one amino acid or a precursor thereof, and/or at least one polyol is metabolised by a cancer-associated microorganism.

It will be appreciated that “prognosis” may relate to determining the therapeutic outcome in a subject that has been diagnosed with cancer. Prognosis may relate to predicting the rate of progression or improvement and/or the duration of cancer in a subject, the probability of survival, and/or the efficacy of various treatment regimes. Thus, a poor prognosis may be indicative of cancer progression, low probability of survival and reduced efficacy of a treatment regime. A favourable prognosis may be indicative of cancer improvement, high probability of survival and increased efficacy of a treatment regime.

The cancer-associated microorganism may be a bacterium. It will be appreciated that the microorganisms and bacteria present in the gut form the so-called “microbiome”.

Therefore, the cancer-associated microorganism that metabolises the at least one substrate into a signature compound, which is detected and/or analysed in the methods of the invention to diagnose cancer, preferably form part of the microbiome.

The cancer-associated microorganism may be Streptococcus, Lactobacillus, Veillonella, Prevotella, Neisseria, Haemophilus, L. coleohominis, Lachnospiraceae, Klebsiella, Clostridiales, Erysipelotrichales, or any combination thereof.

The cancer-associated microorganism may be S. pyogenes, Klebsiella pneumoniae, Lactobacillus acidophilus, or any combination thereof.

The cancer-associated microorganism may be E. coli, P. mirabili, B. cepacia, S. pyogenes, Streptococcus salivarius, Actinomyces naeslundii, Lactobacillus fermentum, Streptococcus anginosus, Clostridium bifermentans, Clostridium perfringens, Clostridium septicum, Clostridium sporogenes, Clostridium tertium, Eubacterium lentum, Eubacterium sp., Fusobacterium simiae, Fusobacterium necrophorum, Lactobacillus acidophilus, Peptococcus niger, Peptostreptococcus anaerobius, Peptostreptococcus asaccharolyticus, Peptostreptococcus prevotii, P. aeruginosa, S. aureus, P. mirabilis, E. faecalis, S. pneumoniae, N. meningitides, Acinetobacter baumannii, Bacteroides capillosus, Bacteroides fragilis, Bacteroides pyogenes, Clostridium difficile, Clostridium ramosum, Enterobacter cloacae, Klebsiella pneumoniae, Nocardia sp., Propionibacterium acnes, Propionibacterium propionicum, or any combination thereof. Preferably, the cancer-associated microorganism is E. coli, L. fermentum, S. salivarius, S. anginosus or K. pneumoniae.

In an embodiment, the cancer is oesophago-gastric junction cancer, gastric cancer, oesophageal cancer, oesophageal squamous-cell carcinoma (ESCC), oesophageal adenocarcinoma (EAC). Therefore, in a preferred embodiment, the diagnosis is for diagnosing oesophago-gastric junction cancer, gastric cancer, oesophageal cancer, oesophageal squamous-cell carcinoma (ESCC), or oesophageal adenocarcinoma (EAC). Most preferably, the cancer is oesophago-gastric cancer, such that this condition can be diagnosed or prognosed. The cancer may be metastatic.

Preferably, the cancer is gastric cancer, oesophageal cancer or a metastasised cancer.

In an embodiment, the cancer is pancreatic cancer or colorectal cancer. Accordingly, the diagnosis or prognosis may be for diagnosing or prognosing pancreatic cancer or colorectal cancer.

In a second aspect, there is provided a method for detecting a signature compound in a test subject, the method comprising:
(i) providing the subject with a composition comprising at least one substrate according to the first aspect into a signature compound; and
(ii) detecting the concentration of the signature compound in a bodily sample from the subject.

Preferably, the detection step is performed as according to the first aspect.

In a third aspect of the invention, there is provided a composition comprising at least one sugar and/or at least one amino acid or a precursor thereof and/or at least one polyol present suitable for metabolism into a signature compound, wherein the sugar is present in the composition at a concentration of more than 20,000 mg/100 ml and the amino acid is present in the composition at a concentration of at least 500 mg/ml and the polyol is present in the composition at a concentration of more than 25,000 mg/100 ml, for use in a method of diagnosis or prognosis, preferably of cancer.

Preferably, the composition and cancer is as defined in the first aspect.

In a fourth aspect, there is provided a composition comprising at least one substrate which is suitable for metabolism by a cancer-associated microorganism into a signature compound, for use in the method of the first or the second aspect.

In a fifth aspect, there is provided a kit for diagnosing a subject suffering from cancer, or a pre-disposition thereto, or for providing a prognosis of the subject's condition, the kit comprising:
(a) a composition comprising at least one substrate as defined in the first aspect;
(b) means for determining the concentration of a signature compound in a sample from a test subject; and
(c) a reference for the concentration of the signature compound in a sample from an individual who does not suffer from cancer,
wherein the kit is used to identify an increase or a decrease in the concentration of the signature compound in the bodily sample from the test subject, compared to the reference, thereby suggesting that the subject suffers from cancer, or has a pre-disposition thereto, or provides a negative prognosis of the subject's condition.

Preferably, the composition and cancer is as defined in the first aspect.

Methods of the first and second aspect may comprise administering or having administered, to the subject, a therapeutic agent or putting the subject on a specialised diet or carrying out chemotherapy or chemoradiotherapy, which prevents, reduces or delays progression of cancer.

Thus, in a sixth aspect, there is provided a method of treating a subject suffering from cancer, said method comprising the steps of:
(i) providing the subject with a composition comprising at least one substrate as defined in the first aspect;
(ii) analysing the concentration of a signature compound resulting from metabolism of the at least one substrate in a bodily sample from a test subject and comparing this concentration with a reference for the concentration signature compound in an individual who does not suffer from cancer, wherein an increase or a decrease in the concentration of the signature compound in the bodily sample from the test subject compared to the reference suggests that the subject is suffering from cancer, or has a pre-disposition thereto, or has a negative prognosis; and
(iii) administering or having administered, to the subject, a therapeutic agent or putting the subject on a specialised diet or carrying out chemotherapy or chemoradiotherapy, wherein the therapeutic agent or the specialised diet or chemotherapy or chemoradiotherapy prevents, reduces or delays progression of cancer.

Preferably, the composition and cancer is as defined in the first aspect.

The methods of the invention are useful for monitoring the efficacy of a treatment for the relevant cancer. For example, the treatment for resectable oesophago-gastric cancer may comprise neoadjuvant chemotherapy, or chemoradiotherapy followed by surgery and adjuvant chemotherapy. The treatment for very early stage oesophago-gastric cancer may comprise endoscopic resection. The treatment for advanced oesophago-gastric cancer may comprise palliative chemotherapy. It has recently been shown that cancer-associated microbiome enhances metastasis to the liver (Bullman et al., Science, 2017). Hence, the invention described herein may be used to monitor the response of therapy directed towards the cancer-associated microbiome.

If the cancer is pancreatic cancer, then treatment may comprise administration of chemotherapy, chemoradiotherapy with or without surgery. For example, if the cancer is colorectal cancer, then treatment may comprise administration of chemotherapy, chemoradiotherapy with or without surgery, or endoscopic resection.

In a seventh aspect, there is provided a method for determining the efficacy of treating a subject suffering from cancer with a therapeutic agent or a specialised diet or chemotherapy or chemoradiotherapy, the method comprising:
(i) providing the subject with a composition comprising at least one substrate according to the first aspect; and
(ii) analysing the concentration of the signature compound resulting from metabolism of the at least one substrate in a bodily sample from a test subject, and comparing this concentration with a reference for the concentration of the signature compound in an individual who does not suffer from cancer,
wherein an increase or a decrease in the concentration of the signature compound in the bodily sample from the test subject compared to the reference suggests that the treatment regime with the therapeutic agent or the specialised diet or chemotherapy or chemoradiotherapy is effective or ineffective.

Preferably, the composition and cancer is as defined in the first aspect.

The composition may be an existing composition, foodstuff or drink, which comprises any one of the aforementioned constituents. Preferably, the composition comprises water. The composition of the invention is ingested by the subject. The composition may be solid or fluid, which may be eaten or swallowed. In an embodiment, the composition may be chewable, which results in release of the substrate and it being taken down into the gut. In an embodiment, the composition may be in the form of a capsule that is designed to degrade at a certain position with the gastrointestinal tract, thereby offering targeted release of the at least one substrate. However, the composition is preferably a liquid (i.e. a drink), which may be swallowed, and which may be referred to as an oral stimulant drink (OSD).

Preferably, a sample is taken from the subject, and the signature compound in the bodily sample is then detected. In some embodiments, the concentration of the signature compound is measured.

A signature compound may be any compound that can indicate or correlate with the presence of a microorganism. The signature compounds, which are detected, may be volatile organic compounds (VOCs), which lead to a fermentation profile, and they may be detected in the bodily sample by a variety of techniques. In one embodiment, these compounds may be detected within a liquid or semi-solid sample in which they are dissolved. In a preferred embodiment, however, the compounds are detected from gases or vapours. For example, as the signature compounds are VOCs, they may emanate from, or form part of, the sample, and may thus be detected in gaseous or vapour form.

An increase or a decrease in the concentration of these signature compounds compared to the reference, suggests that the subject is suffering from cancer, or has a pre-disposition thereto, or provides a negative prognosis of the subject's condition. Preferably, an increase in the concentration of these signature compounds compared to the reference, suggests that the subject is suffering from cancer, or has a pre-disposition thereto, or provides a negative prognosis of the subject's condition.

The VOCs may be short chain fatty acids, aldehydes, alcohols or any combination thereof.

The VOCs may be a C₁-C₃ aldehyde, a C₁-C₃ alcohol, a C₂-C₁₀ alkane wherein a first carbon atom is substituted with the ═O group and a second carbon atom is substituted with an —OH group, a C₁-C₂₀ alkane, a C₄-C₁₀ alcohol, a C₁-C₆ carboxylic acid, a C₄-C₂₀ aldehyde, phenol optionally substituted with a C₁-C₆ alkyl group, a C₂ aldehyde, a C₃ aldehyde, a C₈ aldehyde, a C₉ aldehyde, a C₁₀ aldehyde, a C₁₁ aldehyde, an analogue or derivative of any aforementioned species, or any combination thereof.

The C₁-C₆ carboxylic acid may be selected from the group consisting of formic acid, acetic acid, propanoic acid, butanoic acid, pentanoic acid and hexanoic acid. The C₁-C₃ aldehyde may be selected from the group consisting of formaldehyde, acetaldehyde and propanal. The C₄-C₂₀ aldehyde may be a C₄-C₁₀ aldehyde. C₄-C₂₀ aldehyde may be selected from the group consisting of butanal, pentanal, hexanal, heptanal, octanal, nonanal, decanal, undecanal, dodecanal, tridecanal, tetradecanal, pentradecanal, hexadecanal, heptadecanal, octadecanal, nonadecanal and icodanal. The C₁-C₂₀ alkane is preferably a C₄-C₁₆ alkane, and more preferably a C₈-C₁₄ alkane. The C₁-C₂₀ alkane may be methane, ethane, propane, butane, pentane, hexane, heptane, octane, nonane, decane, undecane, dodecane, tridecane, tetradecane, pentradecane, hexadecane, heptadecane, octadecane, nonadecane and icodane. The phenol may be unsubstituted. Alternatively, the phenol may be substituted with a C₁-C₆ alkyl group in the trans position. The phenol may be substituted with a C₁-C₃ alkyl group. The phenol optionally substituted with a C₁-C₆ alkyl group may be phenol, 1-hydroxy-4-ethylbenzene or P-cresol.

Preferably, the volatile organic compound (VOC) is selected from a group consisting of: acetic acid, butanoic acid, hexanoic acid, pentanoic acid, propanoic acid, acetaldehyde, decanal, heptanal, hexanal, nonanal, octanal, pentanal, butanal, propanal, 1-hydroxy-4-ethylbenzene, decane, dodecane, P-cresol and phenol or any combination thereof.

When the substrate is a sugar, preferably glucose, the signature compound may be acetic acid, butanoic acid, pentanoic acid, propanoic acid, hexanoic acid, acetaldehyde, propanal, butanal, hexanal, pentanal, decanal, 1-hydoxytheylbenzene and/or P-cresol.

When the substrate is a sugar, preferably glucose, an increase in acetic acid, butanoic acid, pentanoic acid, propanoic acid, acetaldehyde, butanal, hexanal, pentanal, 1-hydoxytheylbenzene and/or P-cresol may be indicative of gastric cancer. Preferably, when the substrate is glucose and the signature compound is butanoic acid, the increase in the concentration of the signature compound is an at least 300% increase in the concentration of butanoic acid compound when compared to the reference and is indicative of gastric cancer. Preferably, when the substrate is glucose and the signature compound is propanoic acid, the increase in the concentration of the signature compound is an at least 100% increase in the concentration of propanoic acid compound when compared to the reference and is indicative of gastric cancer. Preferably, when the substrate is glucose and the signature compound is acetic acid, the increase in the concentration of the signature compound is an at least 200% increase in the concentration of acetic acid compound when compared to the reference and is indicative of gastric cancer. Preferably, when the substrate is glucose and the signature compound is pentanoic acid, the increase in the concentration of the signature compound is an at least 50% increase in the concentration of pentanoic acid compound when compared to the reference and is indicative of gastric cancer.

When the substrate is a sugar, preferably glucose, an increase in acetic acid, pentanoic acid, propanoic acid, butanal, propanal, and/or hexanoic acid may be indicative of oesophageal cancer. Preferably, when the substrate is glucose and the signature compound is butanoic acid, propanoic acid and/or acetic acid, the increase in the concentration of the signature compound is an at least 50% increase in the concentration of butanoic acid, propanoic acid and/or acetic acid compound when compared to the reference and is indicative of oesophageal cancer.

When the substrate is a sugar, preferably glucose, and in combination with citric acid, an increase in the signature compound butanoic acid, propanoic acid, and/or propanal may be indicative of oesophageal cancer.

When the substrate is a sugar, preferably glucose, and in combination with citric acid, an increase in the signature compound butanoic acid, propanoic acid, and/or propanal may be indicative of gastric cancer.

When the substrate is an amino acid or precursor thereof the signature compound may be butanal, decanal, heptanal, hexanal, phenol, decane, P-cresol, 1-hydoxytheylbenzene and/or dodecane. Preferably, when the substrate is an amino acid or precursor thereof the increase in the concentration of the signature compound is an at least 10%, 20%, 30%, 40%, or 50% increase when compared to the reference.

When the substrate is tyrosine the signature compound may be butanal, decanal, heptanal, hexanal, phenol, decane, P-cresol and/or dodecane. Preferably the signature compound is decanal and/or dodecane. Preferably, when the substrate is tyrosine, the increase in the concentration of the signature compound is an at least 10%, 20%, 30%, 40% or 50% increase when compared to the reference.

When the substrate is tyrosine, an increase in decanal may be indicative of oesophageal cancer.

When the substrate is tyrosine, an increase in dodecane may be indicative of gastric cancer.

When the substrate is phenylalanine, the signature compound may be dodecane, decane, phenol, decanal and/or dodecane.

When the substrate is phenylalanine, an increase in the signature compound decanal, 1-hydoxytheylbenzene, decane, dodecane, p-cresol and/or phenol may be indicative of oesophageal cancer.

When the substrate is phenylalanine, an increase in the signature compound hydoxytheylbenzene, decane, dodecane, p-cresol and/or phenol may be indicative of gastric cancer.

When the substrate is glutamic acid, the signature compound may be propanal, dodecane, phenol and/or butanoic acid.

When the substrate is glutamic acid, an increase in the signature compound propanal, dodecane, phenol and/or butanoic acid may be indicative of oesophageal cancer.

When the substrate is glutamic acid, an increase in the signature compound propanal, dodecane, phenol and/or butanoic acid may be indicative of gastric cancer.

When the substrate is a polyol, preferably glycerol, the signature compound may be butanoic acid, acetic acid, hexanoic acid, pentanoic acid, propanoic acid, butanal, hexanal, pentanal, and/or propanal.

When the substrate is a polyol, preferably glycerol, an increase in the signature compound butanoic acid, acetic acid, hexanoic acid, pentanoic acid, propanoic acid, butanal, hexanal, pentanal and/or propanal may be indicative of oesophageal cancer.

When the substrate is a polyol, preferably glycerol, an increase in the signature compound butanoic acid, acetic acid, hexanoic acid, pentanoic acid, propanoic acid, butanal, hexanal, pentanal and/or propanal may be indicative of gastric cancer.

Preferably, the sample is any bodily sample into which the signature compound is present or secreted. Preferably, the detection or diagnostic method is therefore performed in vitro. The prognostic method, however, may be performed in vivo. For example, the sample may comprise urine, faeces, hair, sweat, saliva, blood, or tears. In one embodiment, the sample may be assayed for the signature compound's levels immediately. Alternatively, the sample may be stored at low temperatures, for example in a fridge or even frozen before the concentration of signature compound is determined. Measurement of the signature compound in the bodily sample may be made on the whole sample or a processed sample, for instance whole blood or processed blood.

In an embodiment, the sample may be a urine sample. It is preferred that the concentration of the signature compound in the bodily sample is measured in vitro from a urine sample taken from the subject. The compound may be detected from gases or vapours emanating from the urine sample. It will be appreciated that detection of the compound in the gas phase emitted from urine is preferred.

It will also be appreciated that “fresh” bodily samples may be analysed immediately after they have been taken from a subject. Alternatively, the samples may be frozen and stored. The sample may then be de-frosted and analysed at a later date.

Most preferably, however, the bodily sample may be a breath sample from the test subject. The sample may be collected by the subject performing exhalation through the mouth, preferably after nasal inhalation. Preferably, the sample comprises the subject's alveolar air. Preferably, the alveolar air is collected over dead space air by capturing end-expiratory breath. VOCs from breath bags are then preferably pre-concentrated onto thermal desorption tubes by transferring breath across the tubes.

The difference in concentration of signature compound, which would indicate cancer in the subject or a predisposition thereto, may be an increase or a decrease compared to the reference. It will be appreciated that the concentration of signature compound in patients suffering from a disease is highly dependent on a number of factors, for example how far the disease has progressed, and the age and gender of the subject. It will also be appreciated that the reference concentration of signature compound in individuals who do not suffer from the disease may fluctuate to some degree, but that on average over a given period of time, the concentration tends to be substantially constant. In addition, it should be appreciated that the concentration of signature compound in one group of individuals who suffer from a disease may be different to the concentration of that compound in another group of individuals who do not suffer from the disease. However, it is possible to determine the average concentration of signature compound in individuals who do not suffer from the cancer, and this is referred to as the reference or ‘normal’ concentration of signature compound. The normal concentration corresponds to the reference values discussed above.

In one embodiment, the methods of the invention preferably comprise determining the ratio of chemicals within the breath (i.e. use other components within it as a reference), and then compare these markers to the disease to show if they are elevated or reduced.

The signature compound is preferably a volatile organic compound (VOC), which provides a profile, and it may be detected in or from the bodily sample by a variety of techniques. Thus, these compounds may be detected using a gas analyser. Examples of suitable detector for detecting the signature compound preferably includes an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

The inventors have demonstrated that the signature compounds can be reliably detected using gas chromatography, mass spectrometry, GCMS or TOF. Dedicated sensors could be used for the detection step.

The reference values may be obtained by assaying a statistically significant number of control samples (i.e. samples from subjects who do not suffer from the disease). Accordingly, the reference (ii) according to the kit of the fifth aspect of the invention may be a control sample (for assaying).

The apparatus preferably comprises a positive control (most preferably provided in a container), which corresponds to the signature compound(s). The apparatus preferably comprises a negative control (preferably provided in a container). In a preferred embodiment, the kit may comprise the reference, a positive control and a negative control. The kit may also comprise further controls, as necessary, such as “spike-in” controls to provide a reference for concentration, and further positive controls for each of the signature compounds, or an analogue or derivative thereof.

Accordingly, the inventors have realised that the difference in concentrations of the signature compound between the reference normal (i.e. control) and increased/decreased levels, can be used as a physiological marker, suggestive of the presence of a disease in the test subject. It will be appreciated that if a subject has an increased/decrease concentration of one or more signature compounds which is considerably higher/lower than the ‘normal’ concentration of that compound in the reference, control value, then they would be at a higher risk of having the disease, or a condition that was more advanced, than if the concentration of that compound was only marginally higher/lower than the ‘normal’ concentration.

The skilled technician will appreciate how to measure the concentrations of the signature compound in a statistically significant number of control individuals, and the concentration of compound in the test subject, and then use these respective figures to determine whether the test subject has a statistically significant increase/decrease in the compound's concentration, and therefore infer whether that subject is suffering from the disease for which the subject has been screened.

The kit of the fifth aspect may comprise sample extraction means for obtaining the sample from the test subject. The sample extraction means may comprise a needle or syringe or the like. The kit may comprise a sample collection container for receiving the extracted sample, which may be liquid, gaseous or semi-solid. The kit may further comprise instructions for use.

In a further aspect, there is provided a method for diagnosing a subject suffering from cancer, or a pre-disposition thereto, or for providing a prognosis of the subject's condition, the method comprising:
(i) detecting, in a bodily sample from a test subject, the concentration of a signature compound resulting from the metabolism of at least one sugar and/or at least one amino acid or a precursor thereof and/or at least one polyol present in a composition previously administered to the subject, wherein the sugar is present in the composition at a concentration of more than 20,000 mg/100 ml, the amino acid or a precursor thereof is present in the composition at a concentration of at least 500 mg/ml and the polyol is present in the composition at a concentration of more than 30,000 mg/100 ml; and
(ii) comparing this concentration with a reference for the concentration of the signature compound in an individual who does not suffer from cancer, wherein an increase or a decrease in the concentration of the signature compound compared to the reference, suggests that the subject is suffering from cancer, or has a pre-disposition thereto, or provides a negative prognosis of the subject's condition.

In another aspect, there is provided a composition comprising at least one sugar and/or at least one amino acid or a precursor thereof and/or at least one polyol present suitable for metabolism into a signature compound, wherein the sugar is present in the composition at a concentration of more than 20,000 mg/100 ml and the amino acid is present in the composition at a concentration of at least 500 mg/ml and the polyol is present in the composition at a concentration of more than 30,000 mg/100 ml, for use in a method of diagnosis or prognosis, preferably of cancer.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figure, in which:

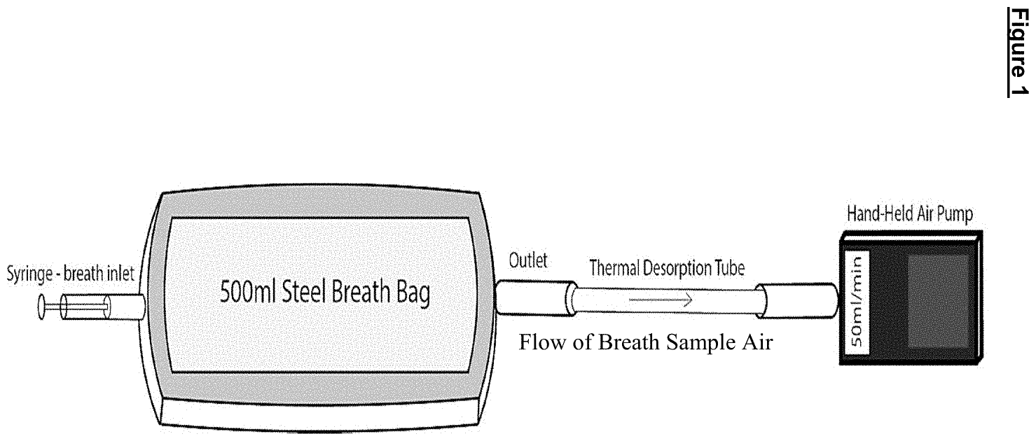

FIG. 1 shows an embodiment of an apparatus and a method used for concentrating VOCs from steel breath bags onto thermal desorption tubes;

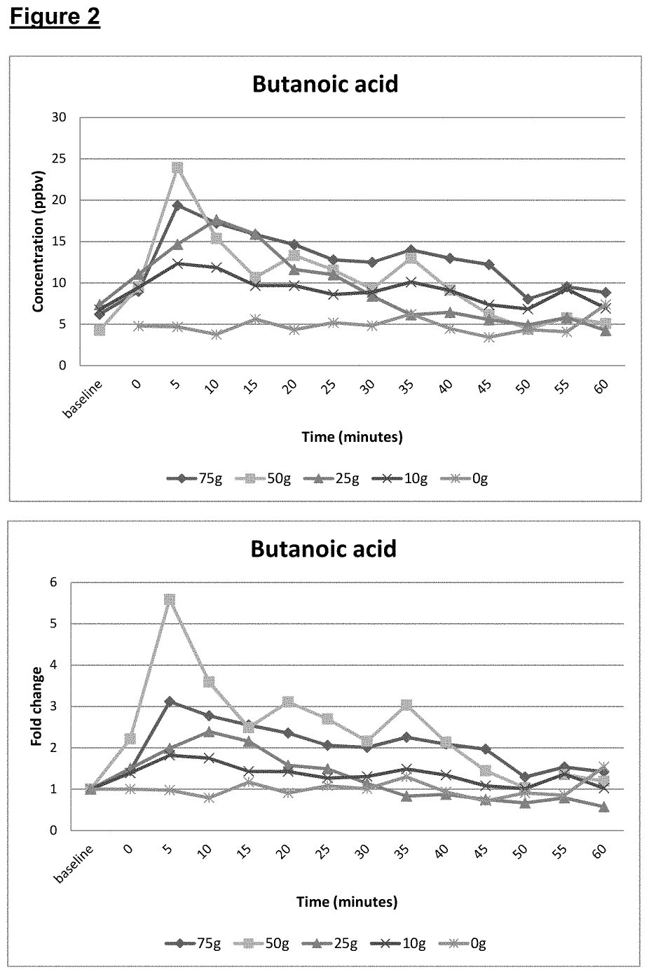

FIG. 2 shows butanoic acid concentrations detected within exhaled breath at varying doses (upper panel shows fold change; lower panel shows concentration (ppbv). Optimal dose responses between 25-75 g of glucose, 5-10 minutes after glucose consumption in subject 1;

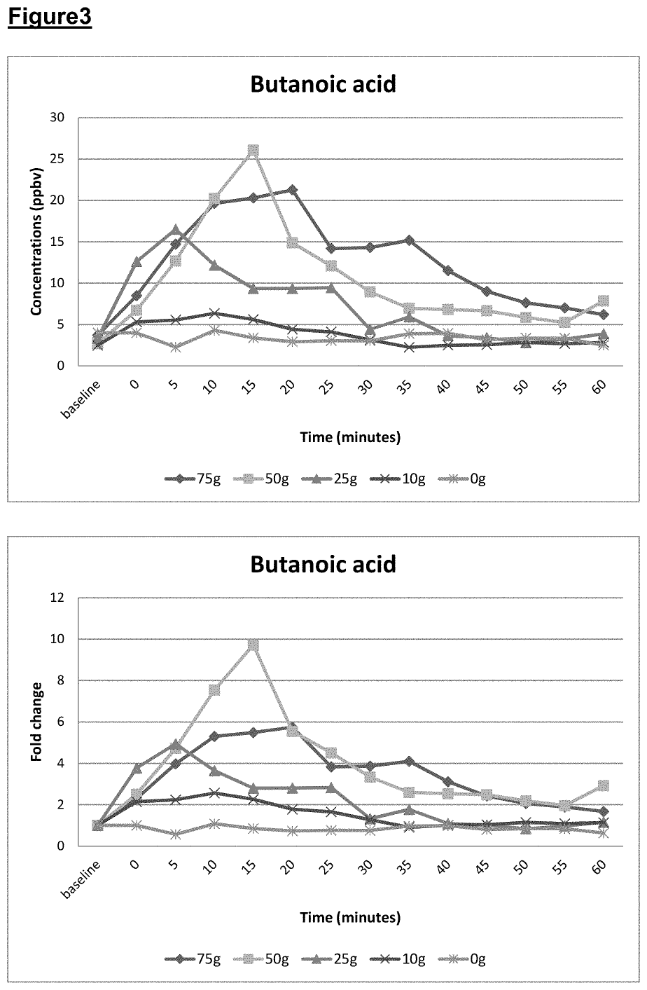

FIG. 3 shows butanoic acid concentrations detected within exhaled breath at varying doses. Optimal dose responses between 25-75 g of glucose, 5-15 minutes after glucose consumption in subject 2;

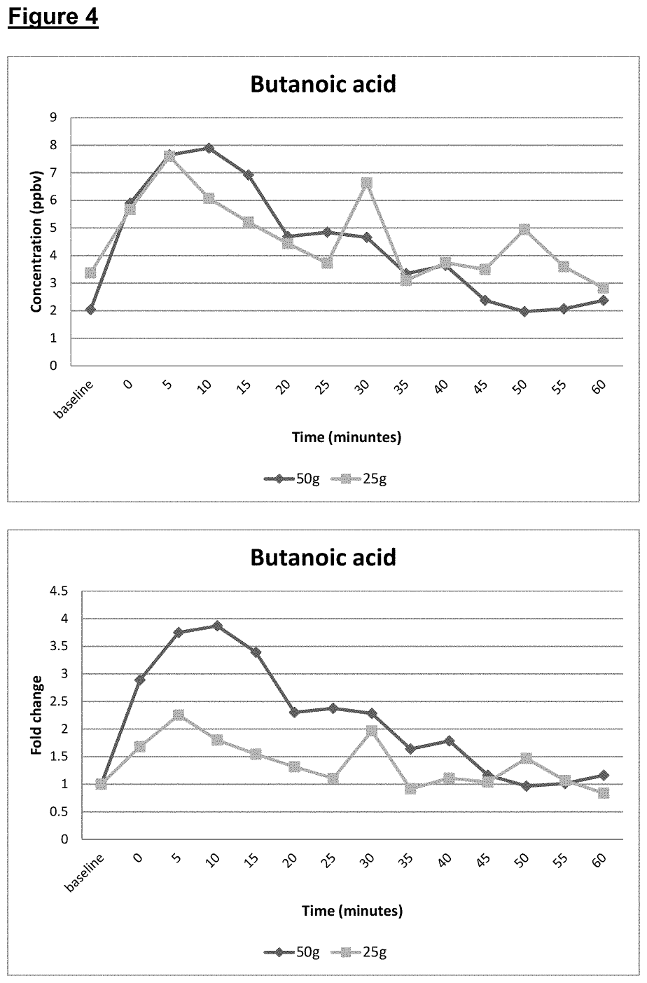

FIG. 4 shows butanoic acid concentrations detected within exhaled breath at varying doses **only 2 doses of 5 completed. Comparable dose responses between 25-50 g of glucose, 5-10 minutes after glucose consumption. 50 g glucose demonstrates approximately double the fold change compared to 25 g glucose in subject 3;

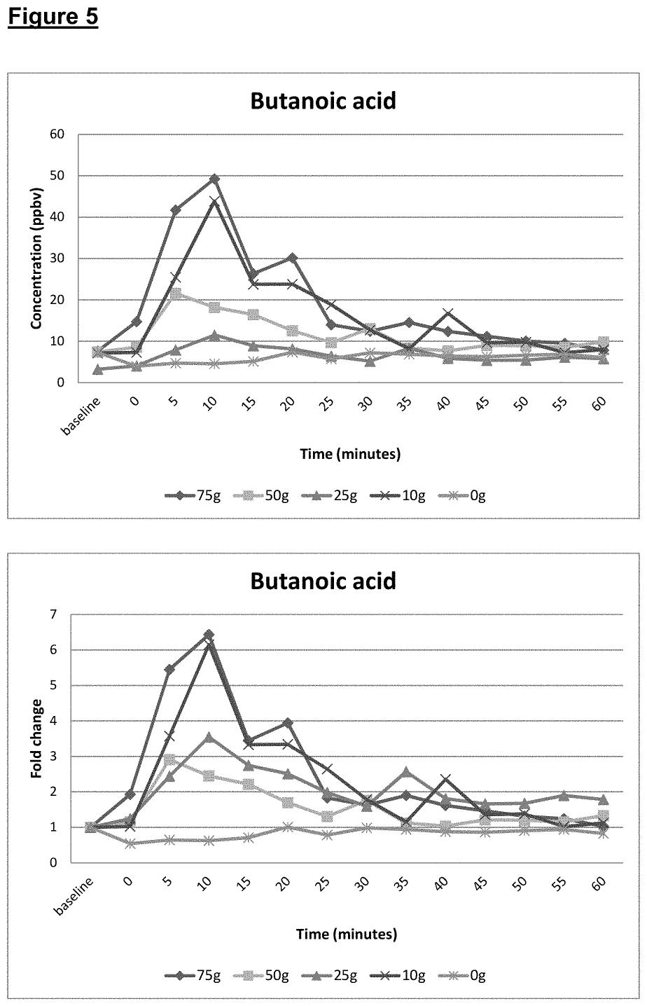

FIG. 5 shows butanoic acid concentrations detected within exhaled breath at varying doses. Optimal dose responses between 10-75 g of glucose, 5-10 minutes after glucose consumption in subject 4;

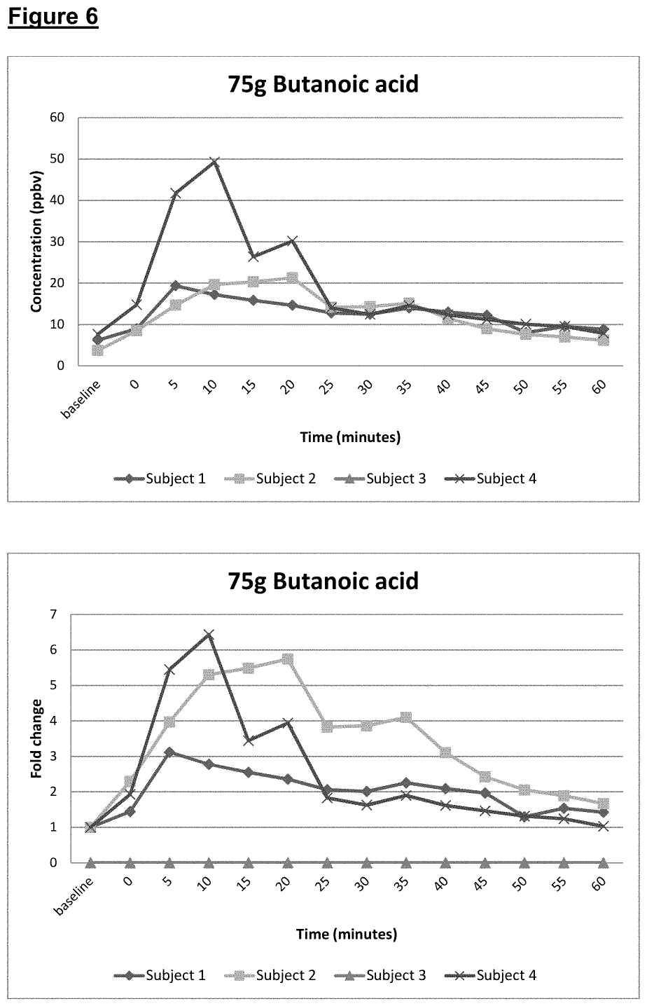

FIG. 6 shows a subject comparison between volatile butanoic acid concentrations within exhaled breath for 75 g glucose (n=3);

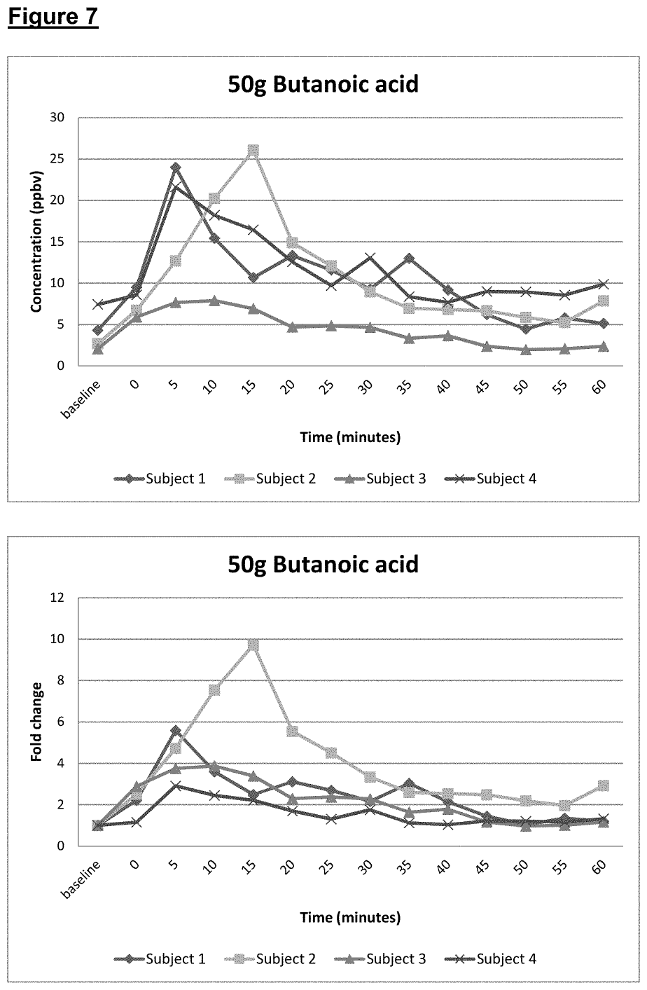

FIG. 7 shows a subject comparison between volatile butanoic acid concentrations within exhaled breath for 50 g glucose (n=4);

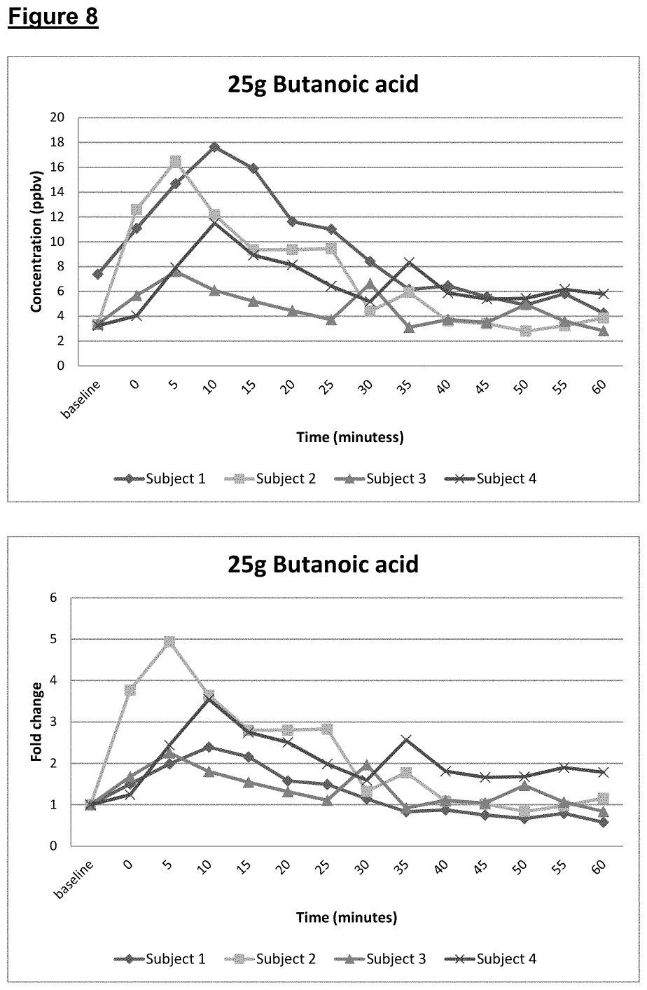

FIG. 8 shows subject comparison between volatile butanoic acid concentrations within exhaled breath for 25 g glucose (n=4);

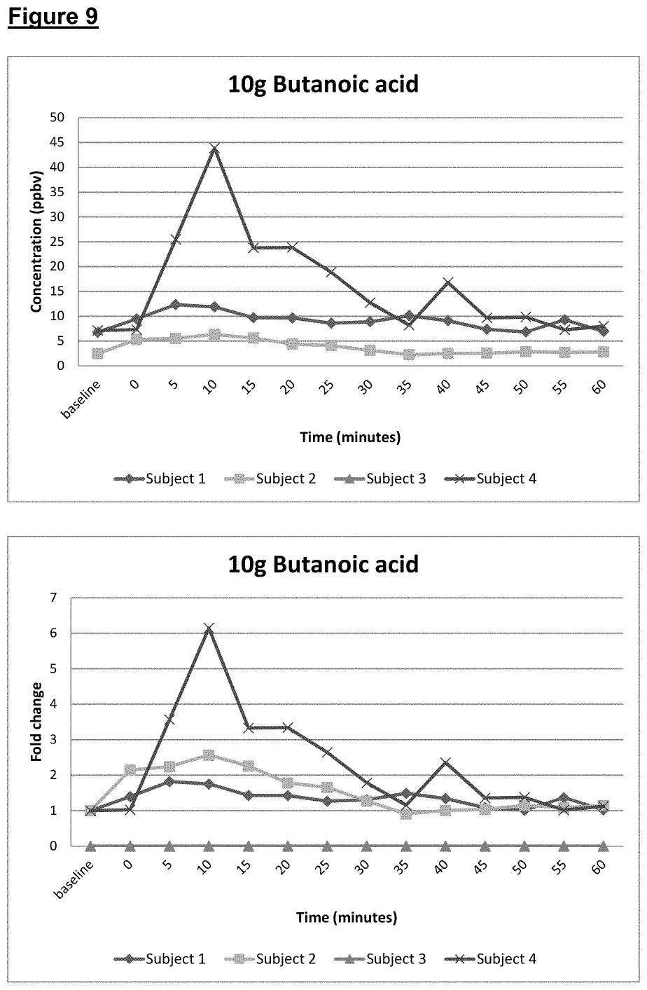

FIG. 9 shows a subject comparison between volatile butanoic acid concentrations within exhaled breath for log glucose (n=3);

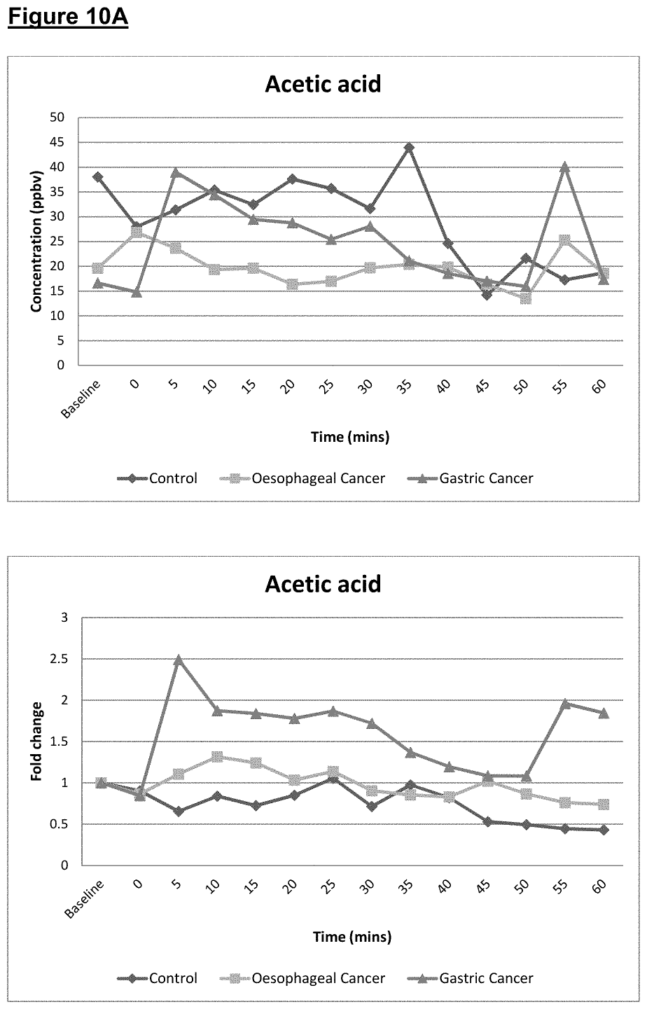

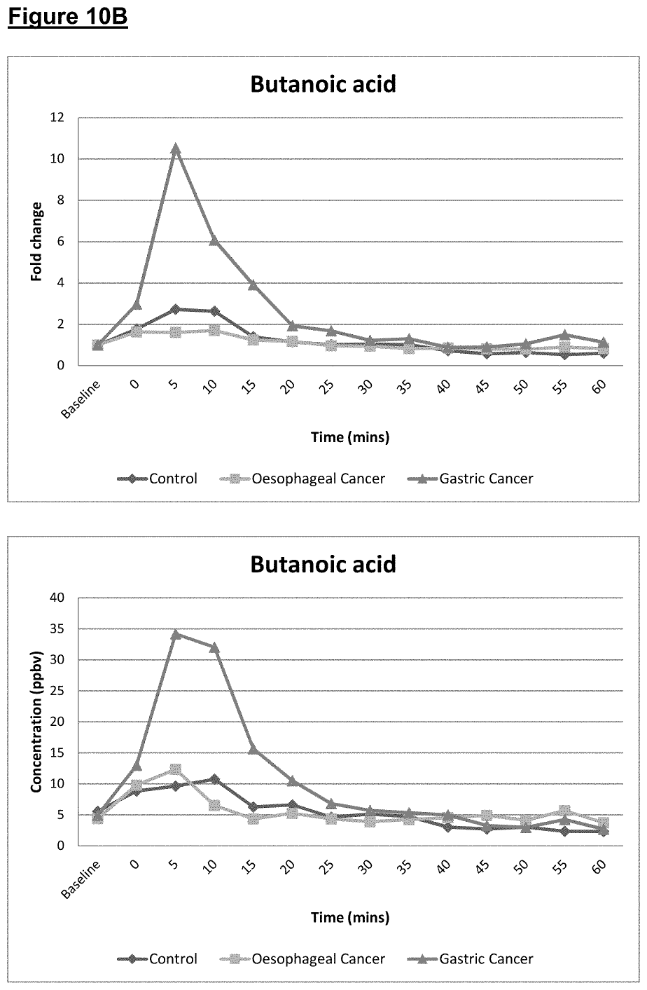

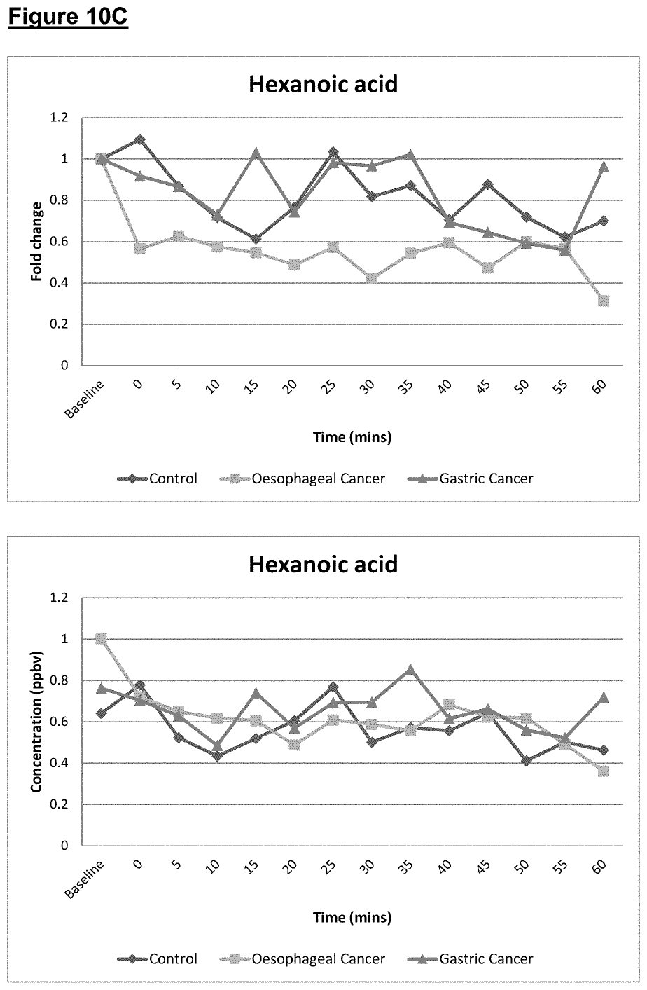

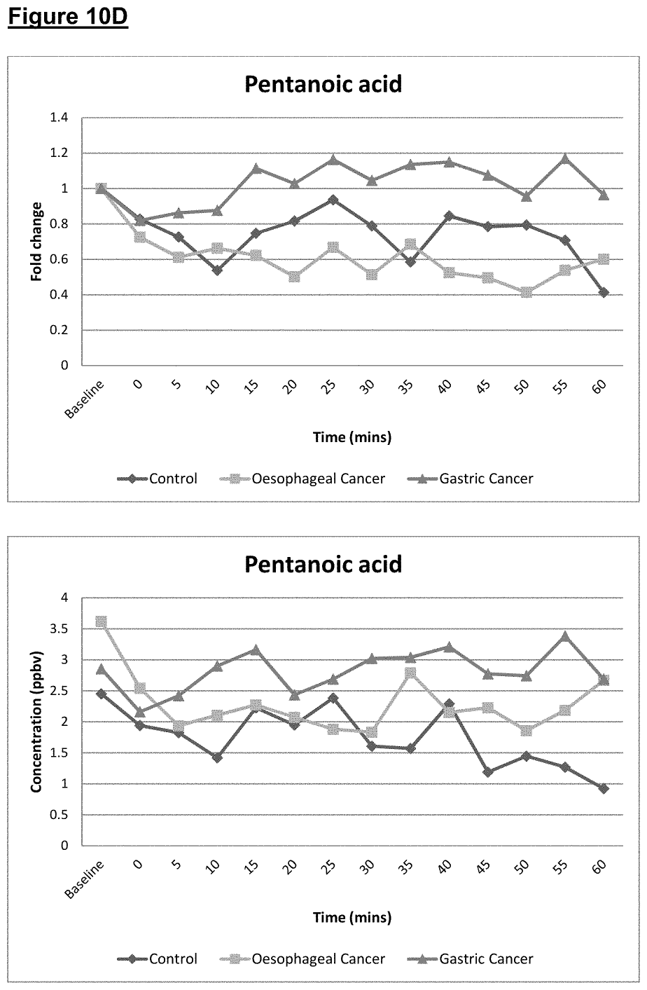

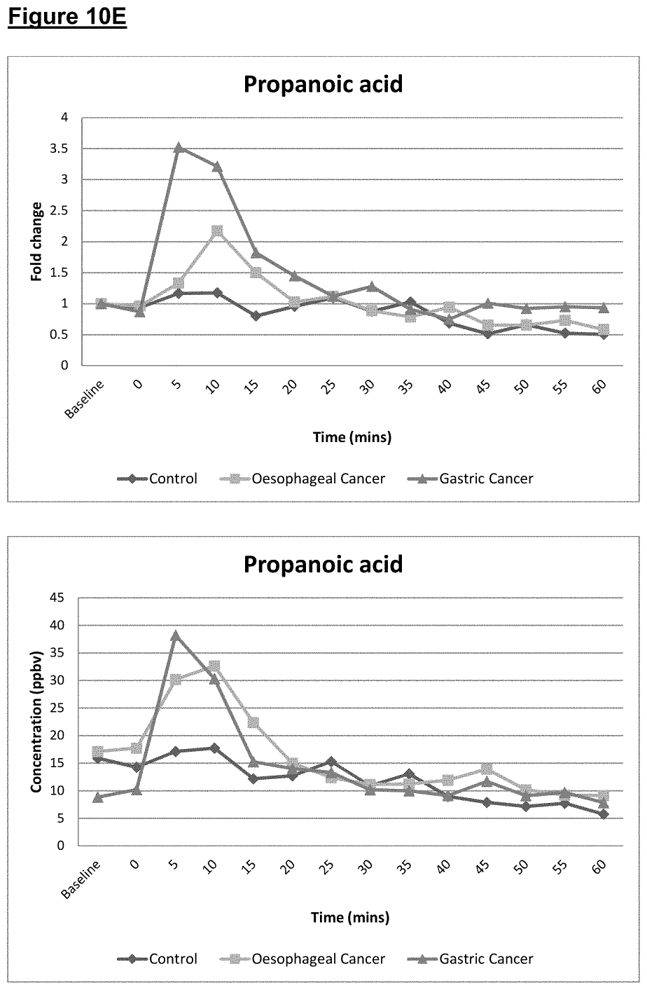

FIG. 10A to 10E shows that of number of the volatile short chain fatty acids tested (acetic, butanoic, hexanoic acid, pentanoic and propanoic acid) increased maximally at 5-10 mins after glucose consumption;

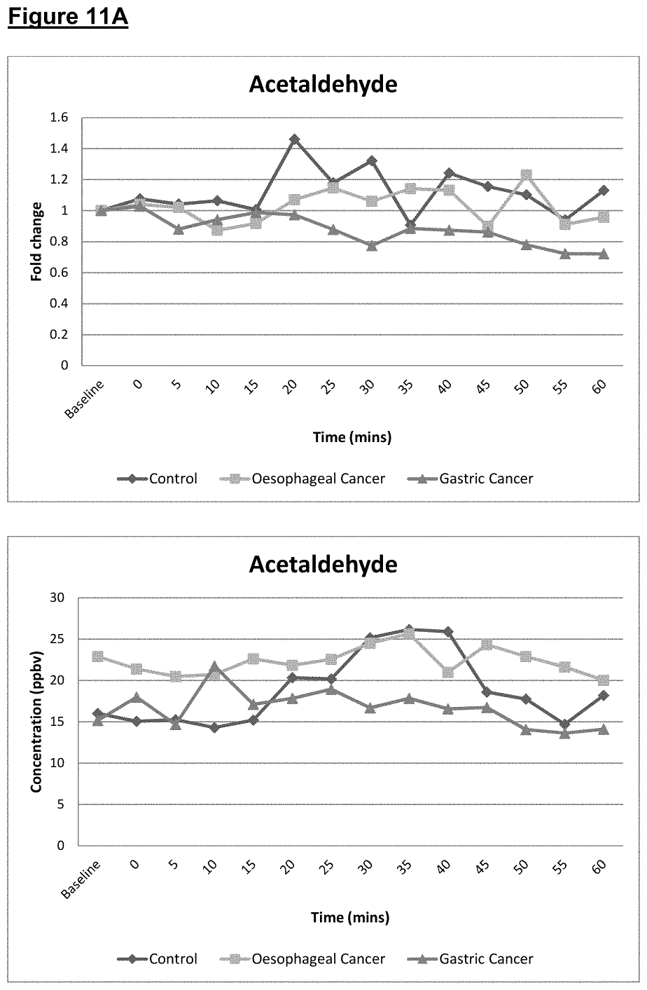

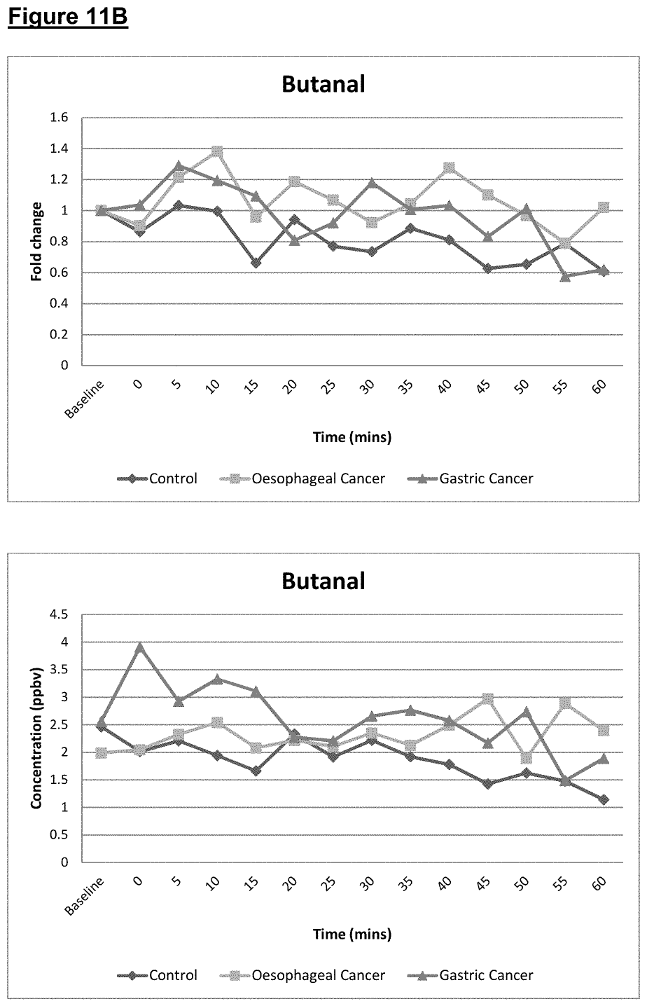

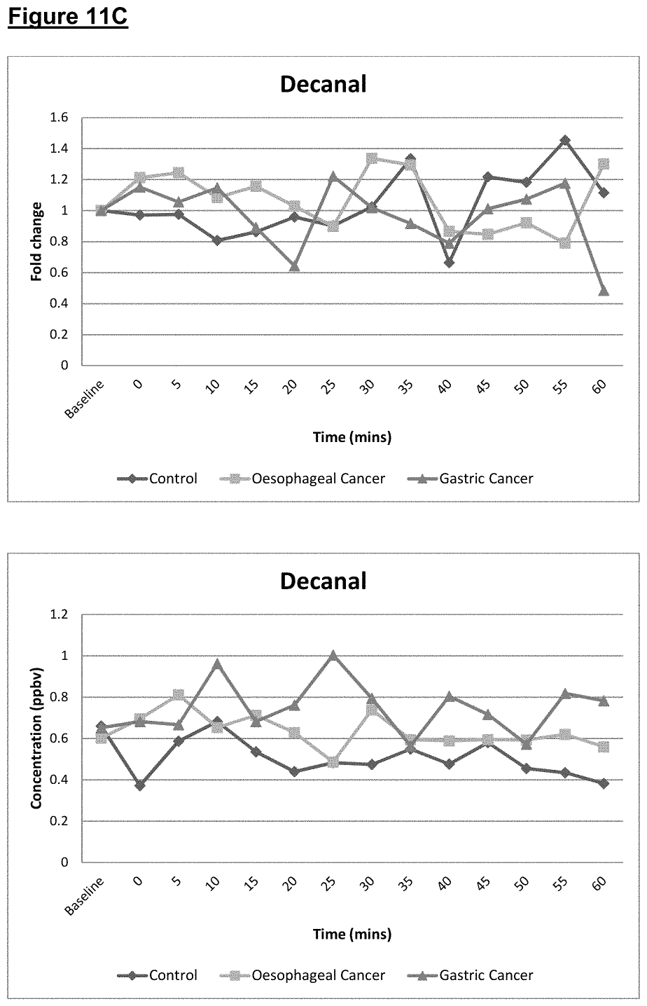

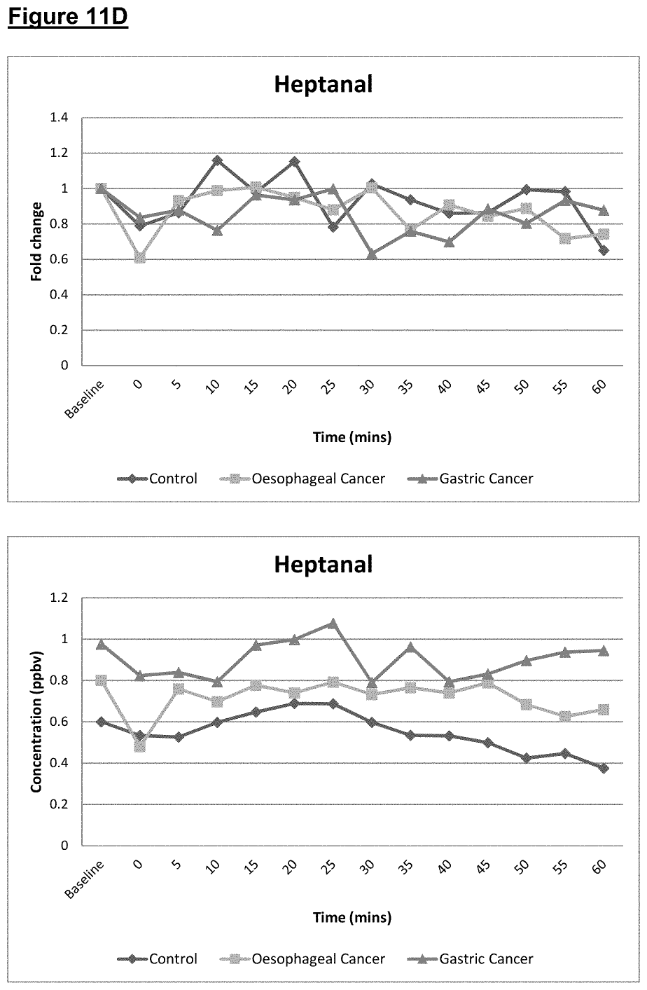

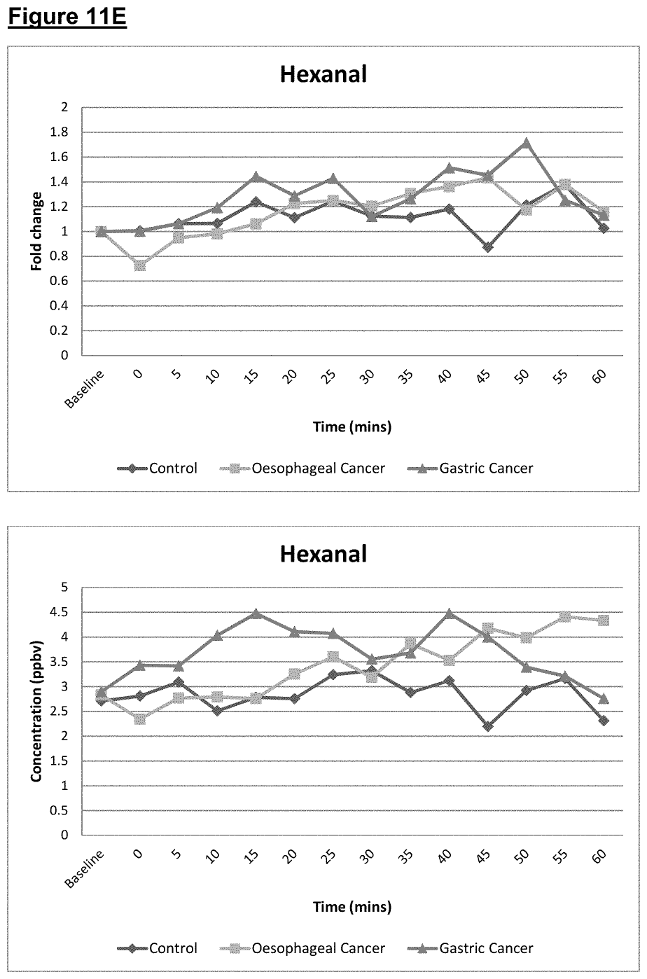

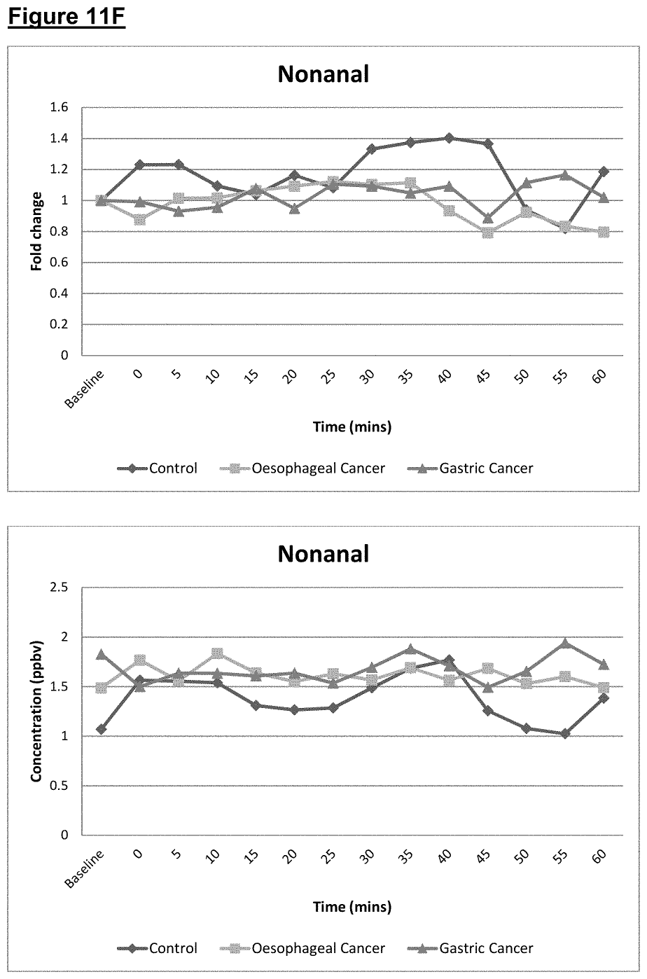

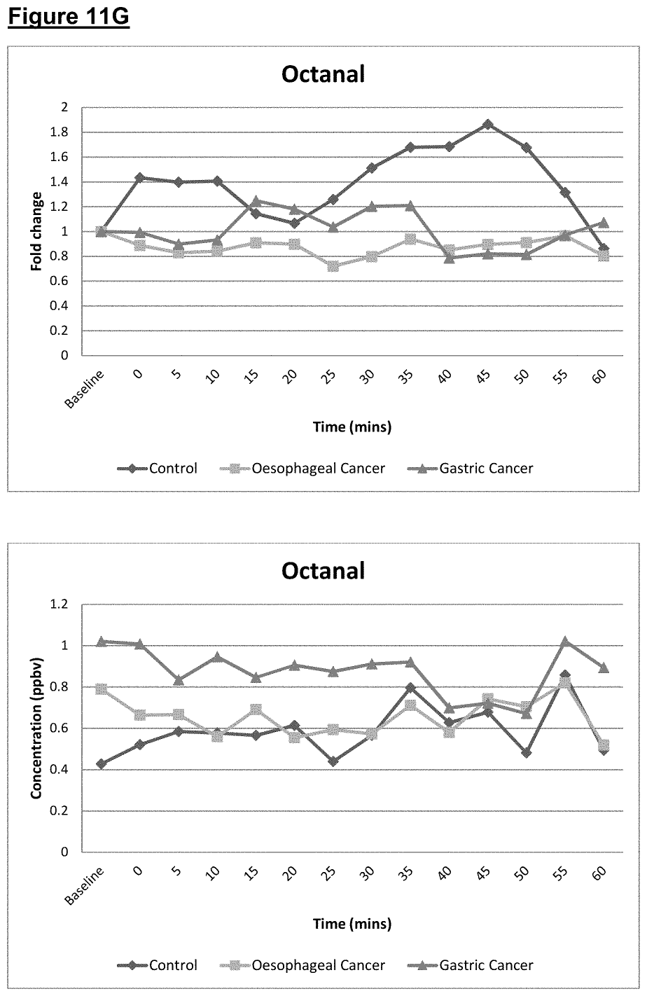

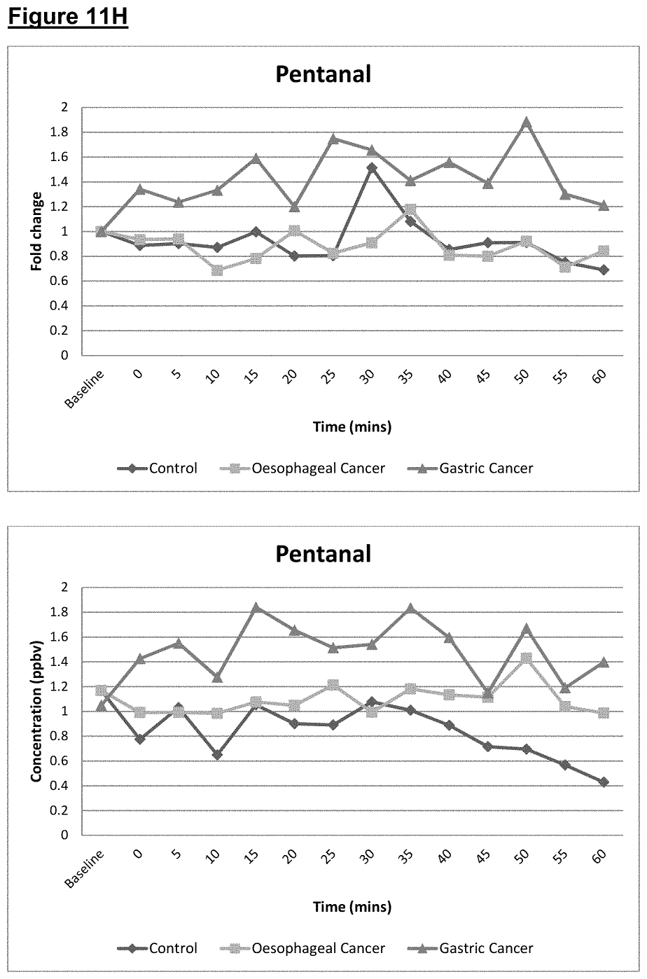

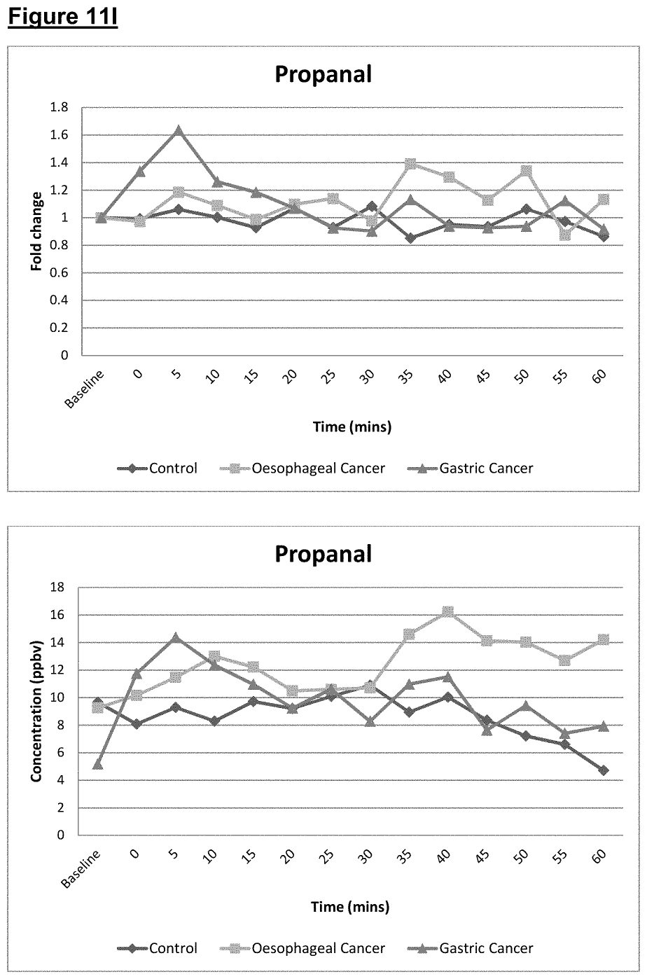

FIG. 11A to 11I shows that a number of the volatile aldehydes tested were maximally increased at 5 minutes (butanal, decanal, propanal) and at 15 minutes (pentanal);

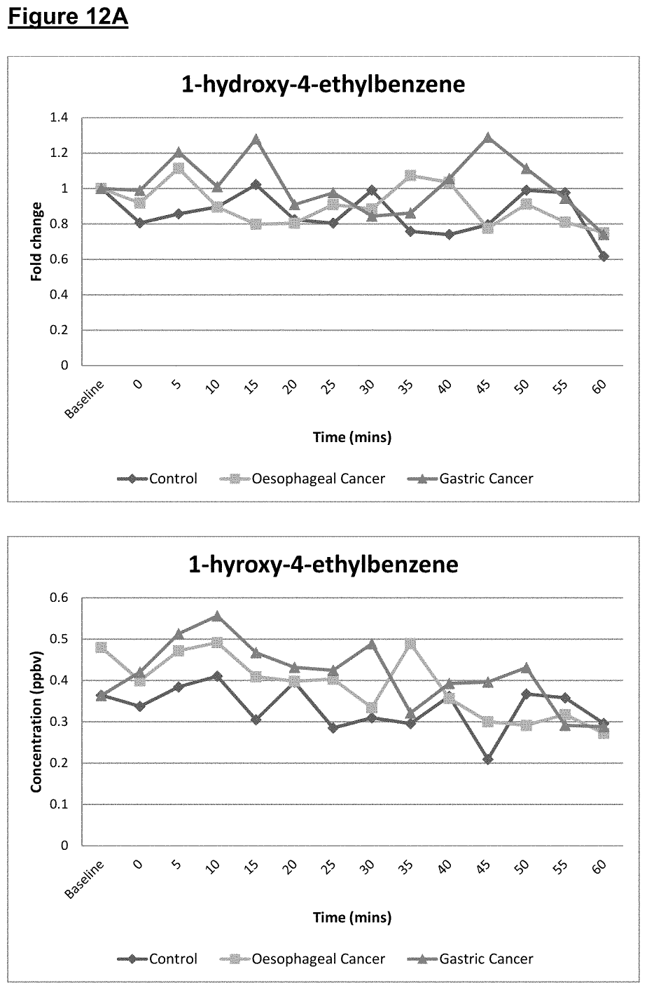

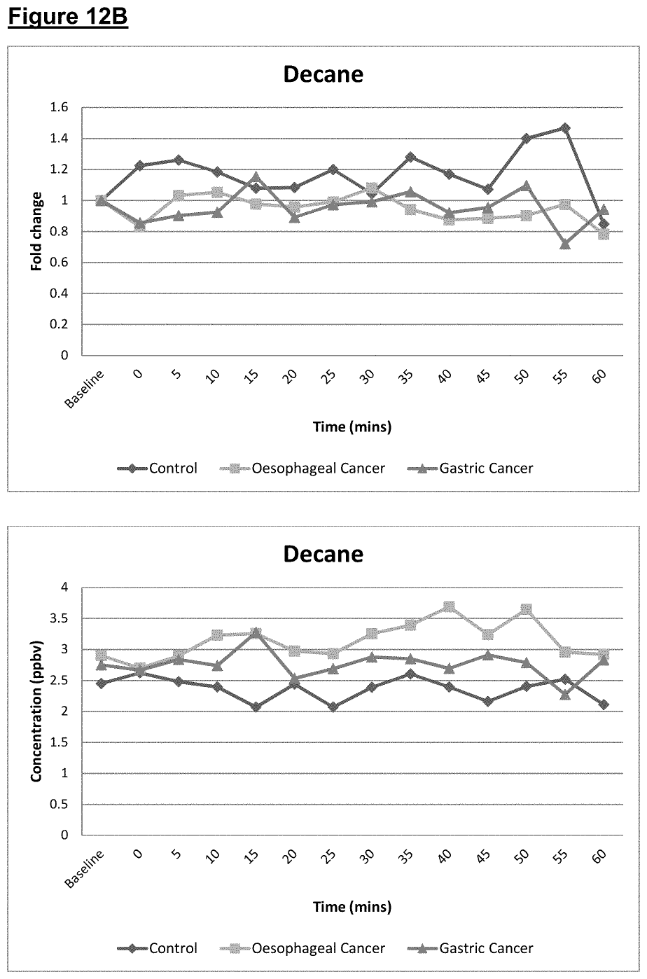

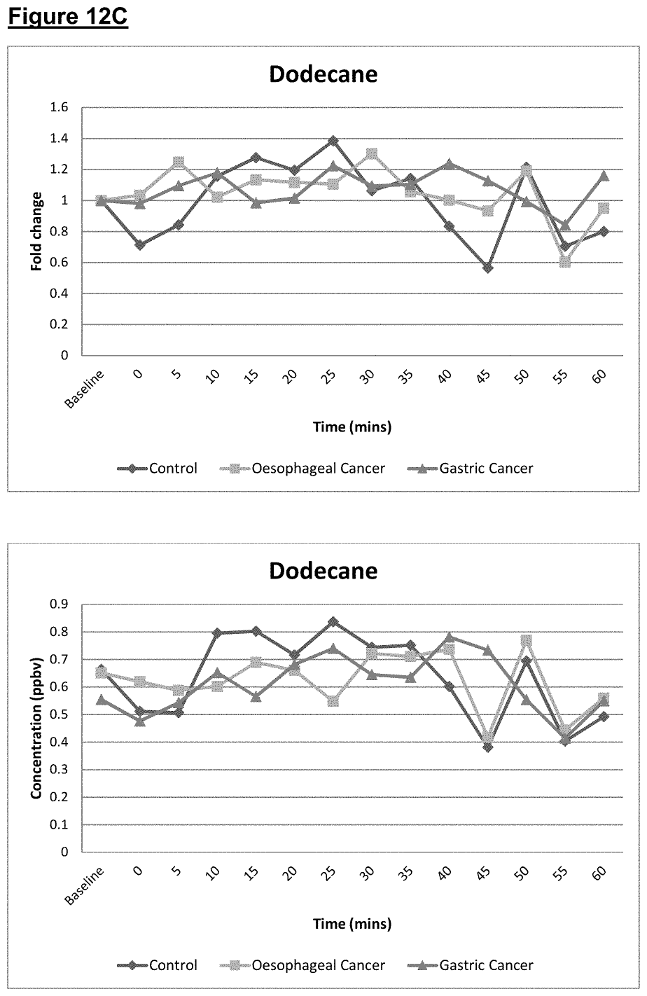

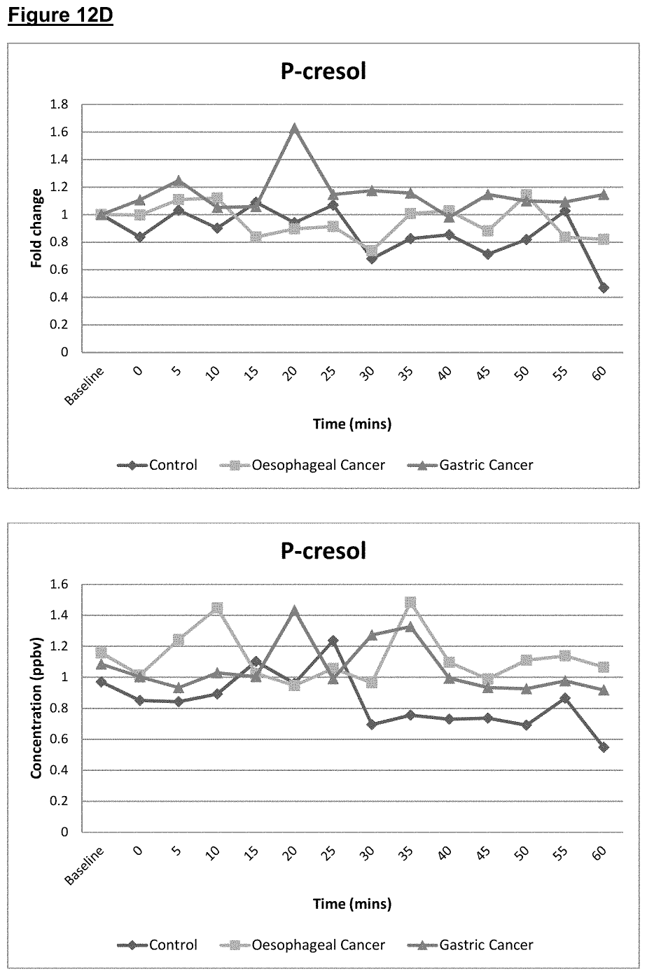

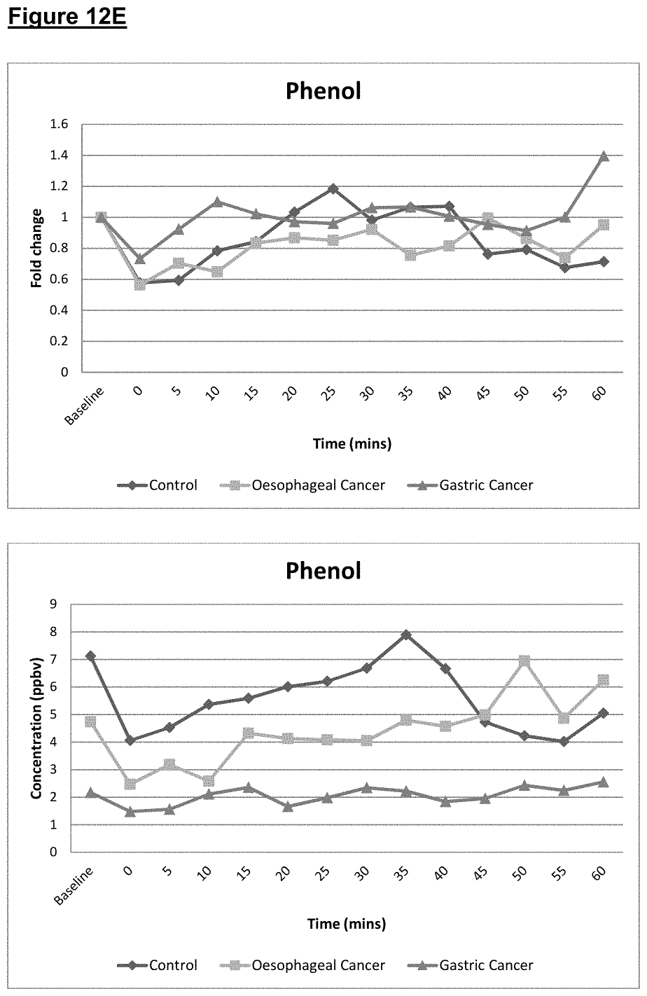

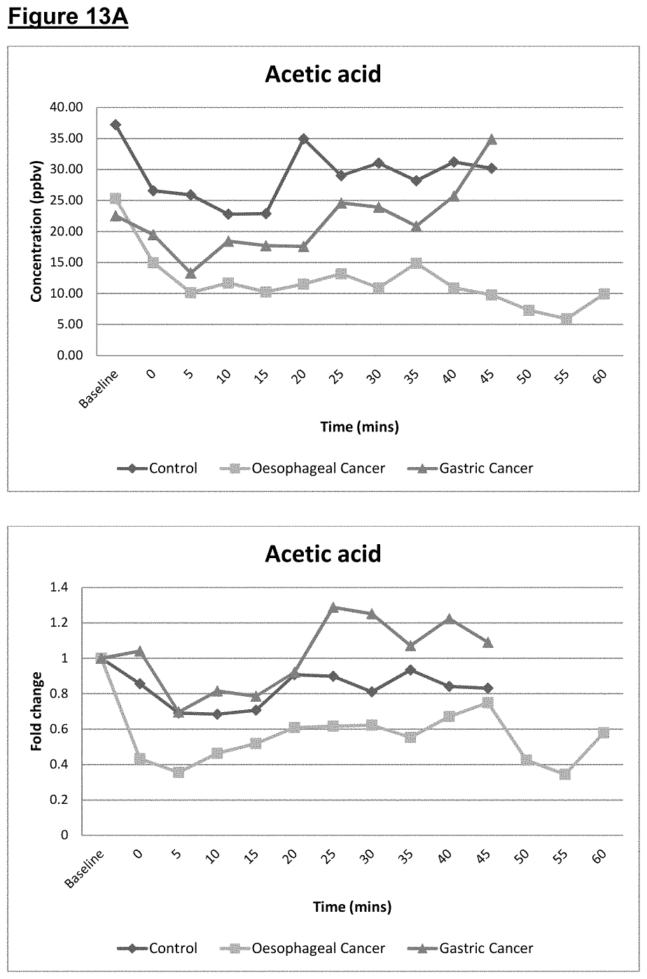

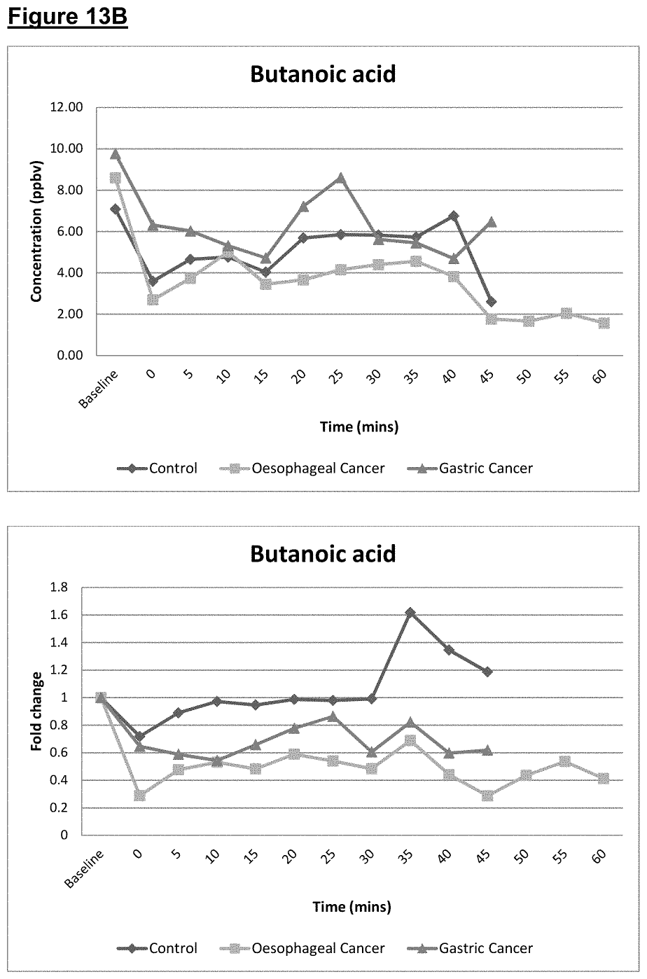

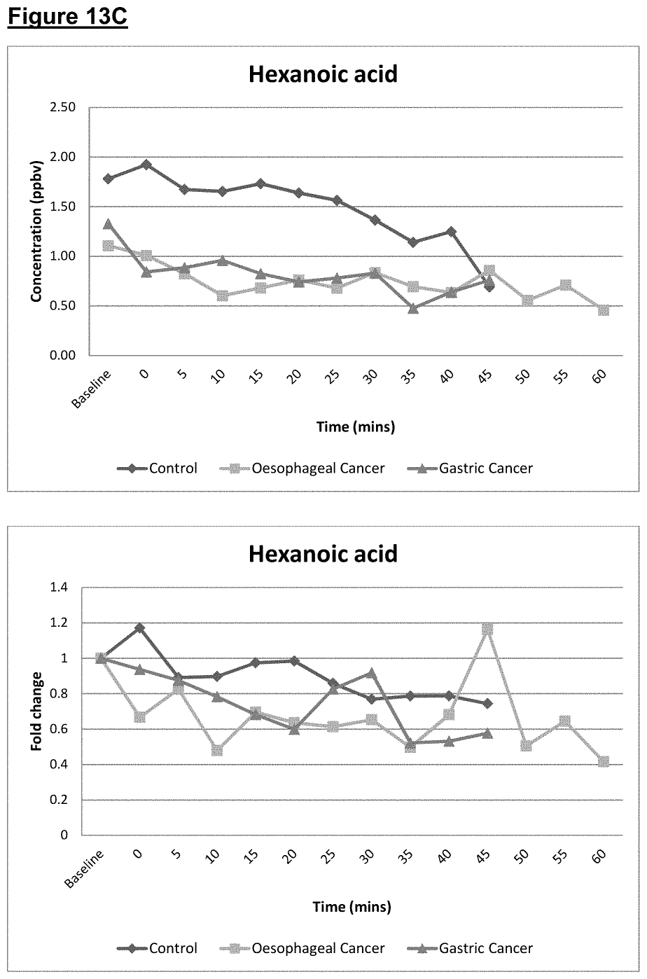

FIG. 12A to 12E shows that a number of the volatile phenols tested demonstrated an increase in exhaled breath concentrations 5 minutes after glucose consumption (1-hydroxy-4-ethylbenzene, dodecane, p-cresol, phenol);

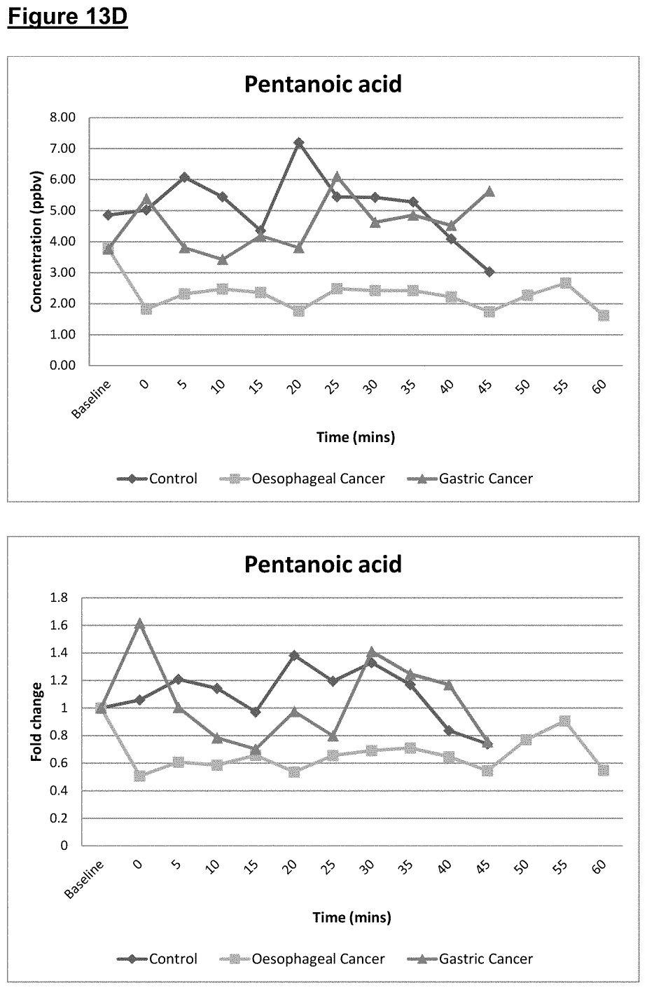

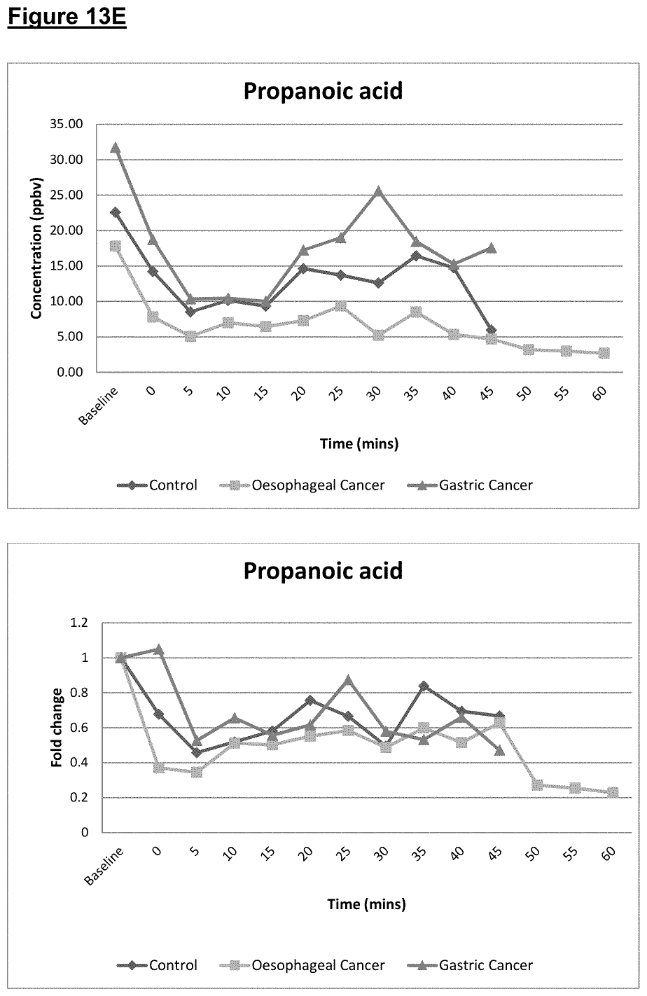

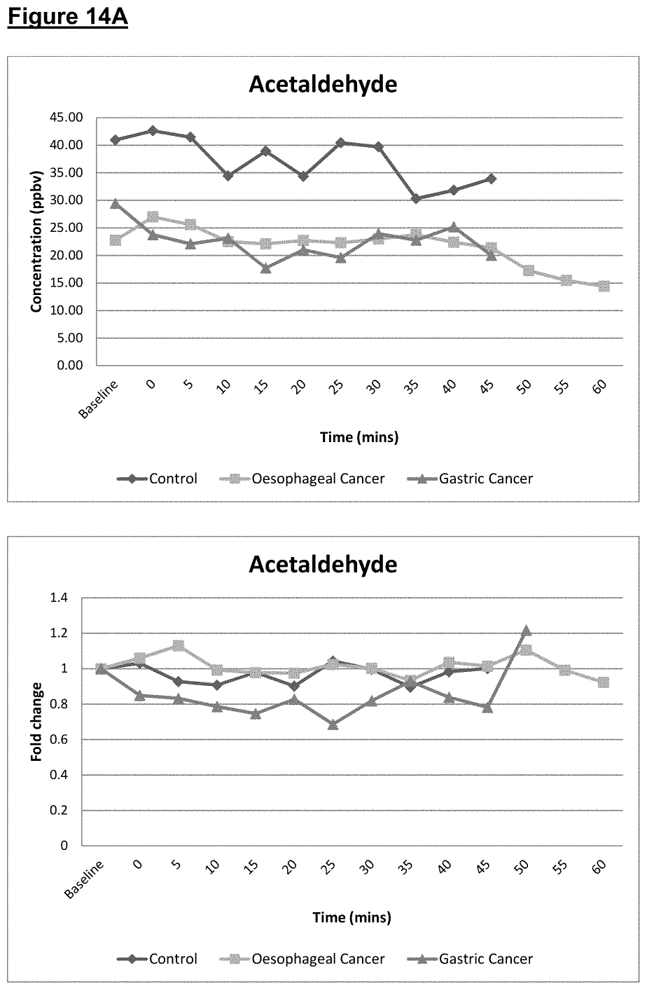

FIG. 13A to 13E shows that the volatile short chain fatty acids tested demonstrated no significant changes after tyrosine consumption;

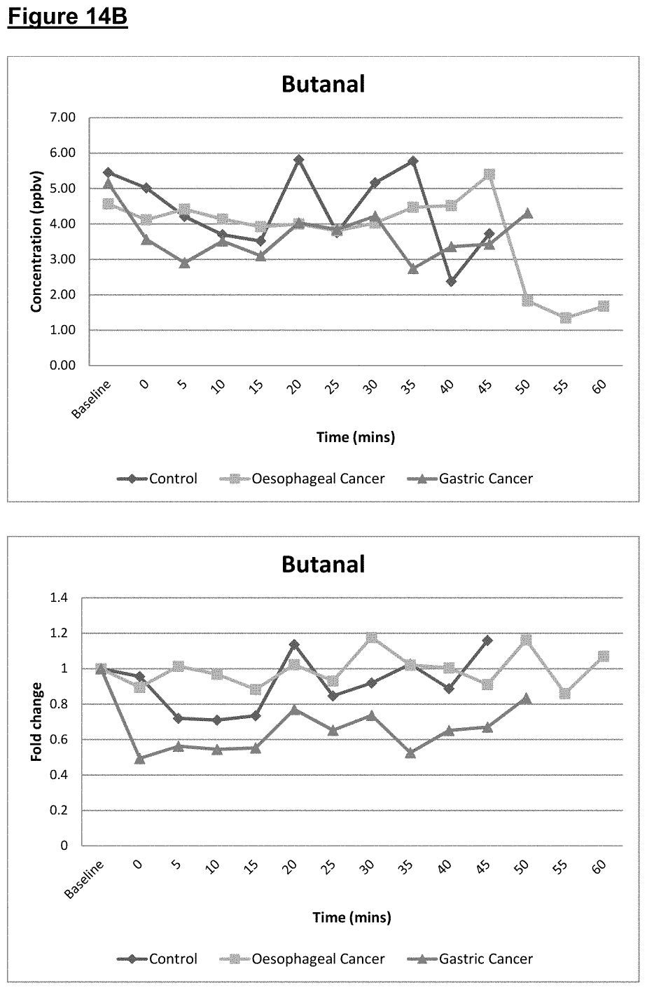

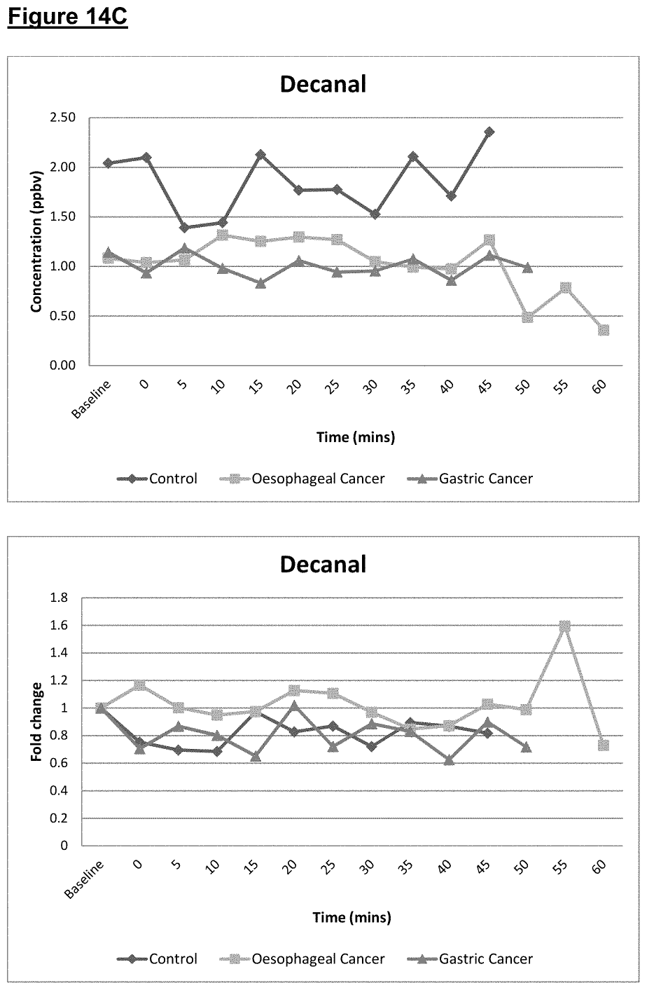

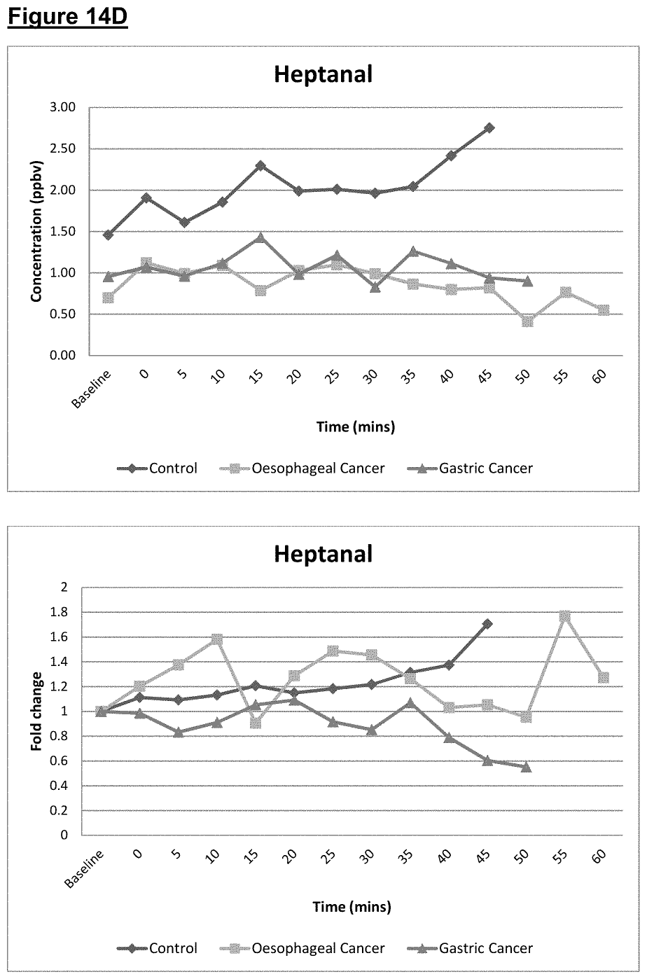

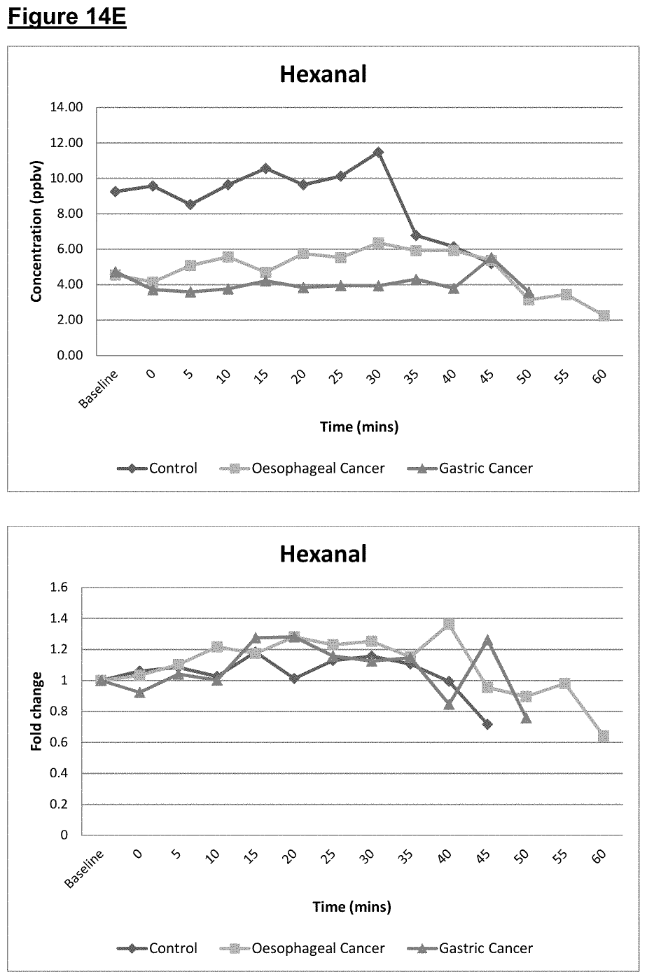

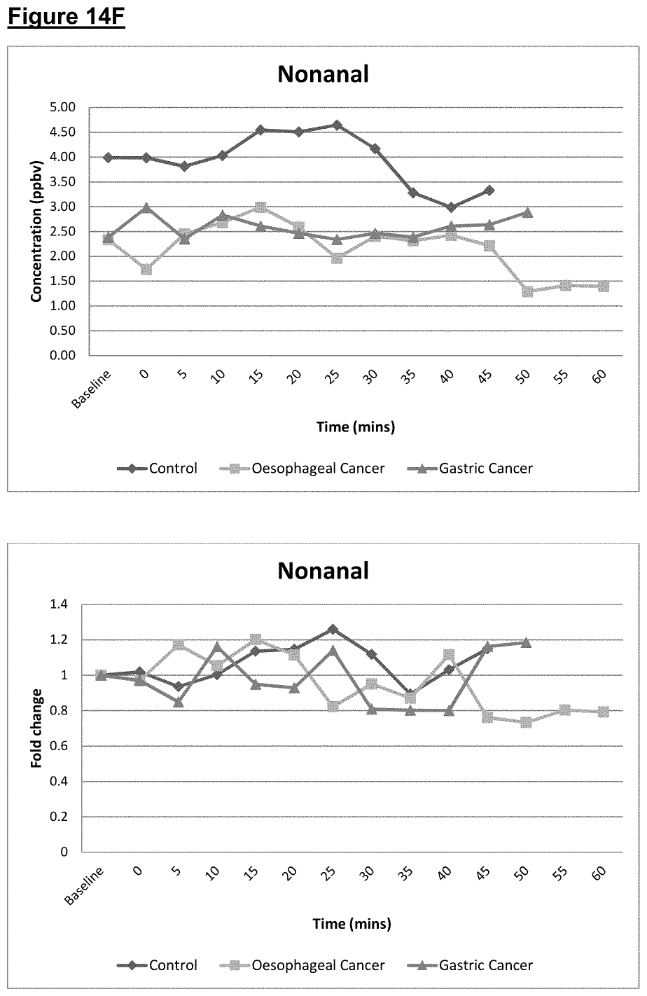

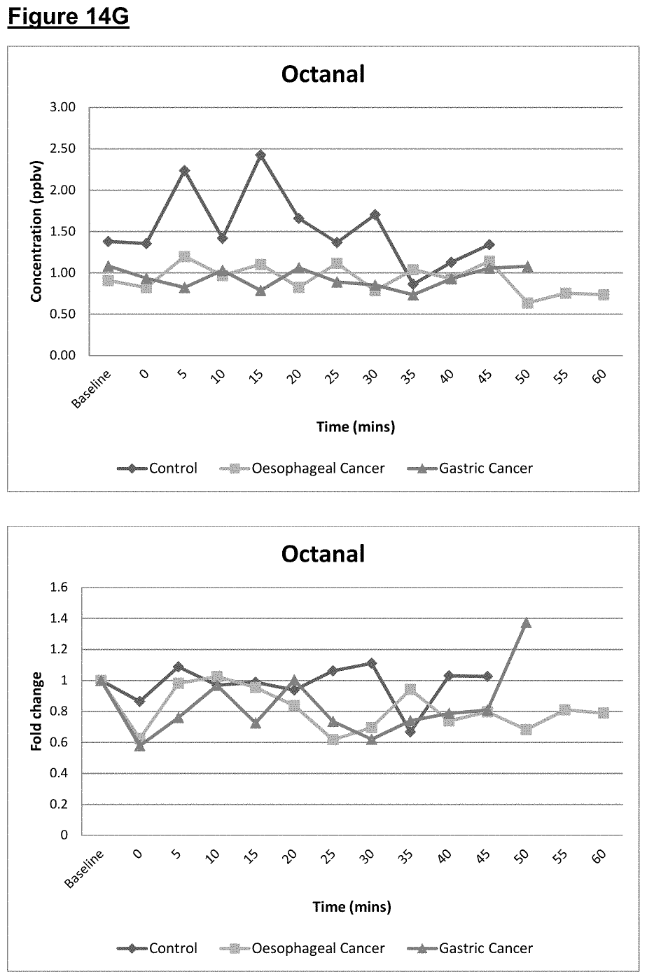

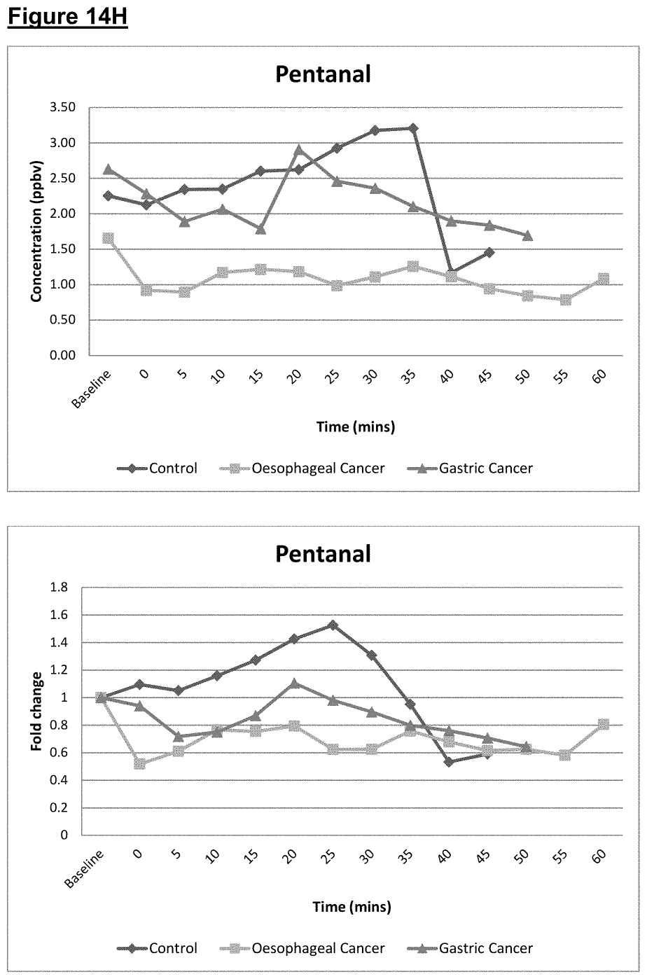

FIG. 14A to 14I shows that a number of the volatile aldehydes tested (butanal, decanal, heptanal and hexanal) demonstrated small increases at approximately 30 minutes after tyrosine ingestion;

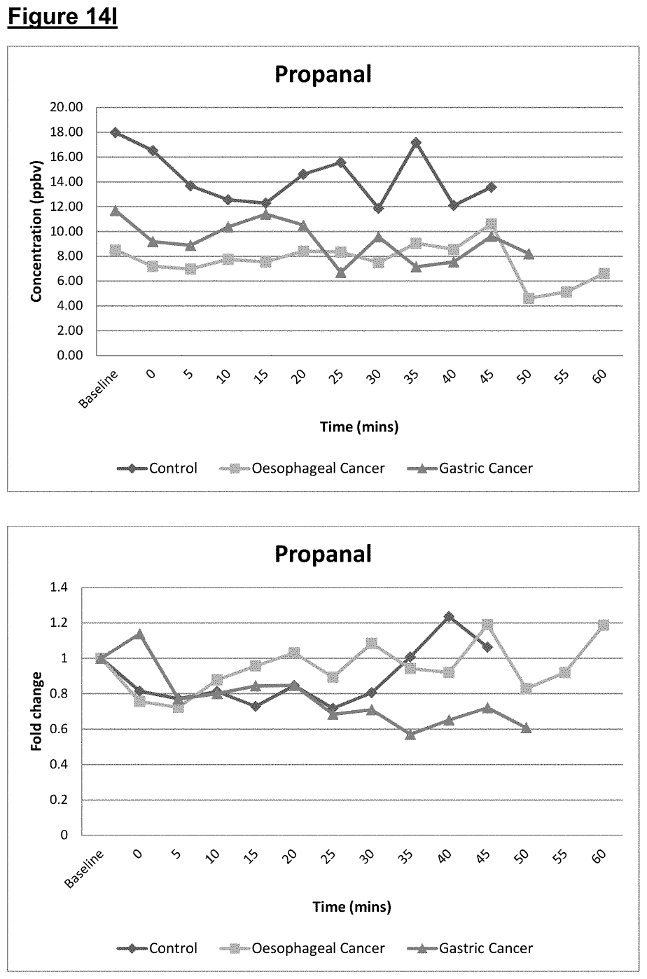

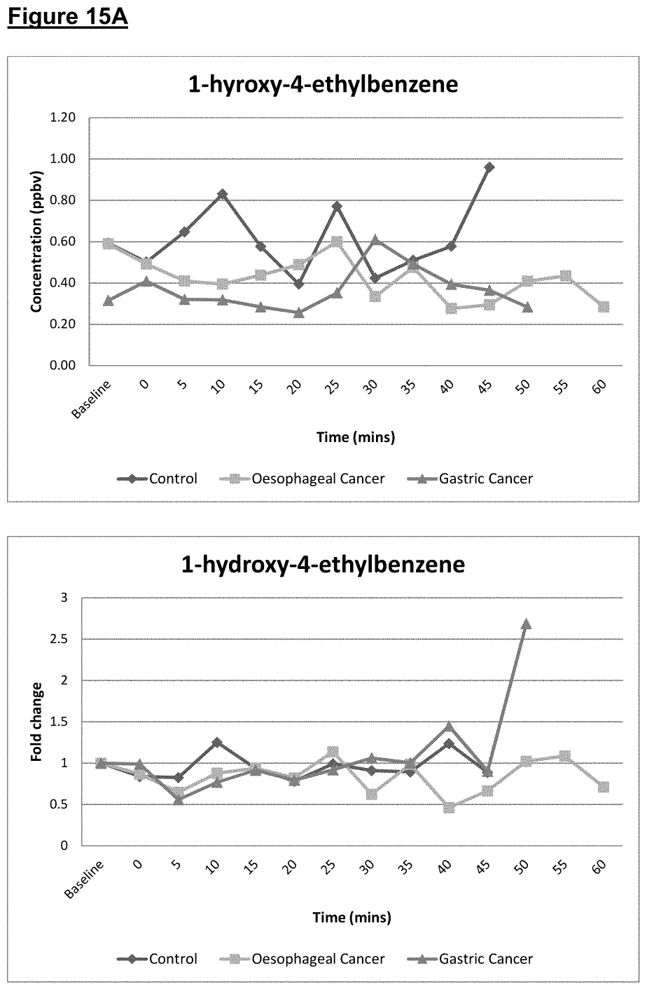

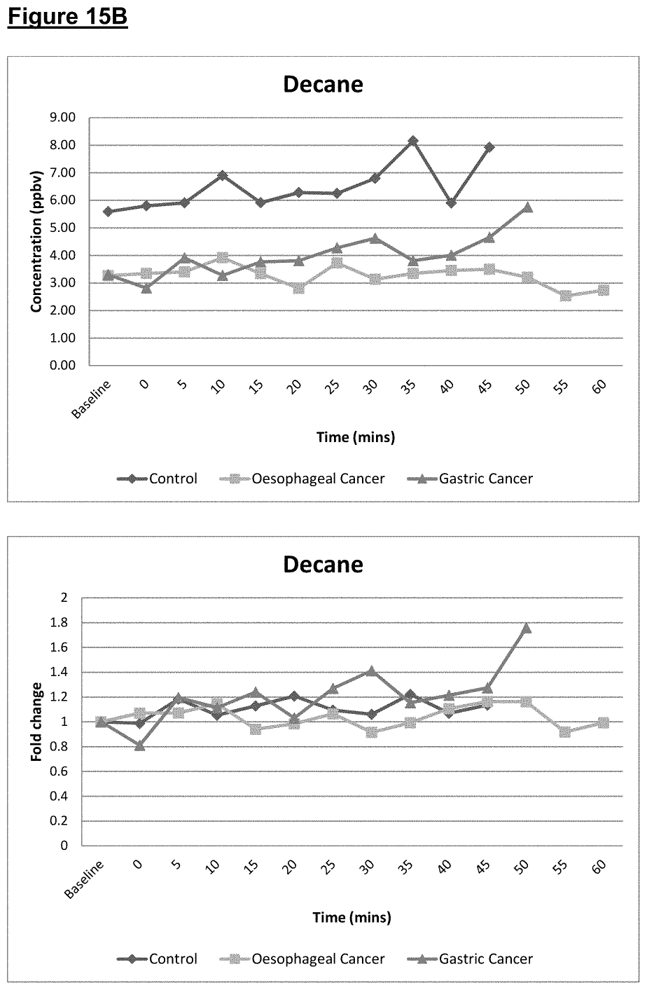

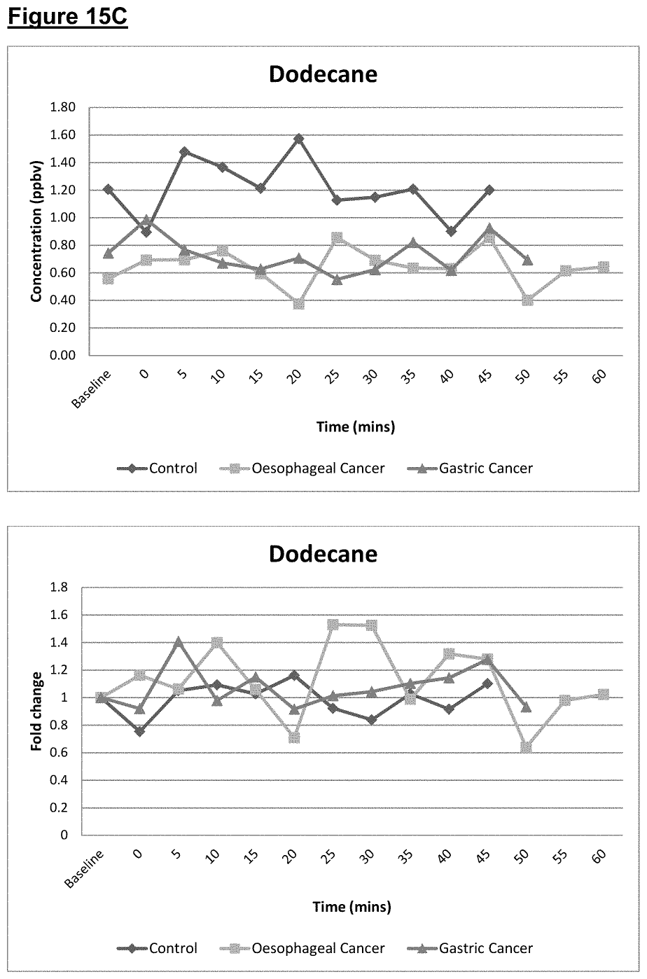

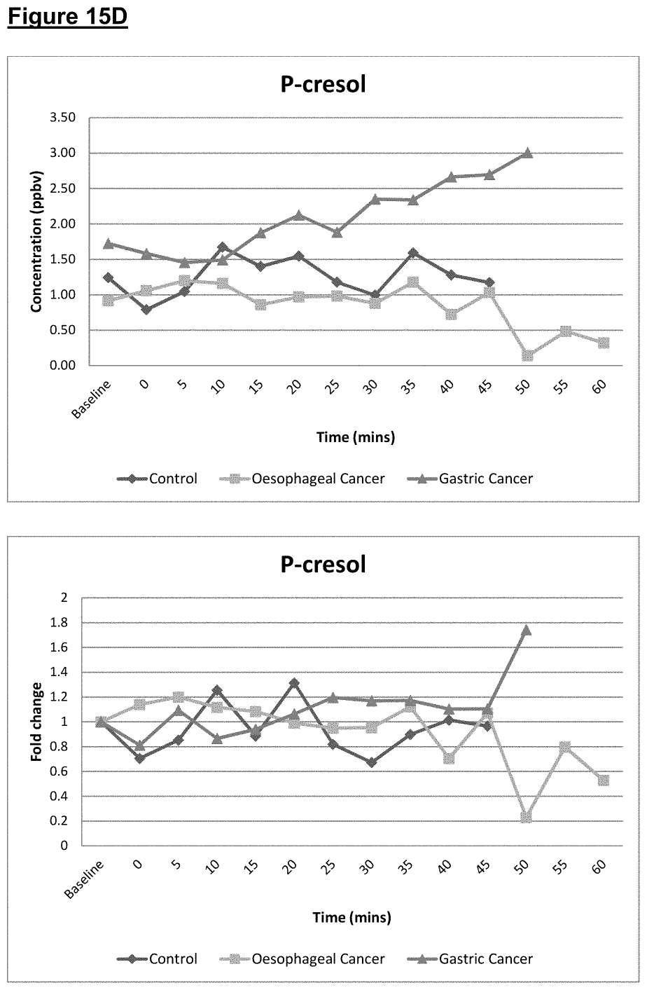

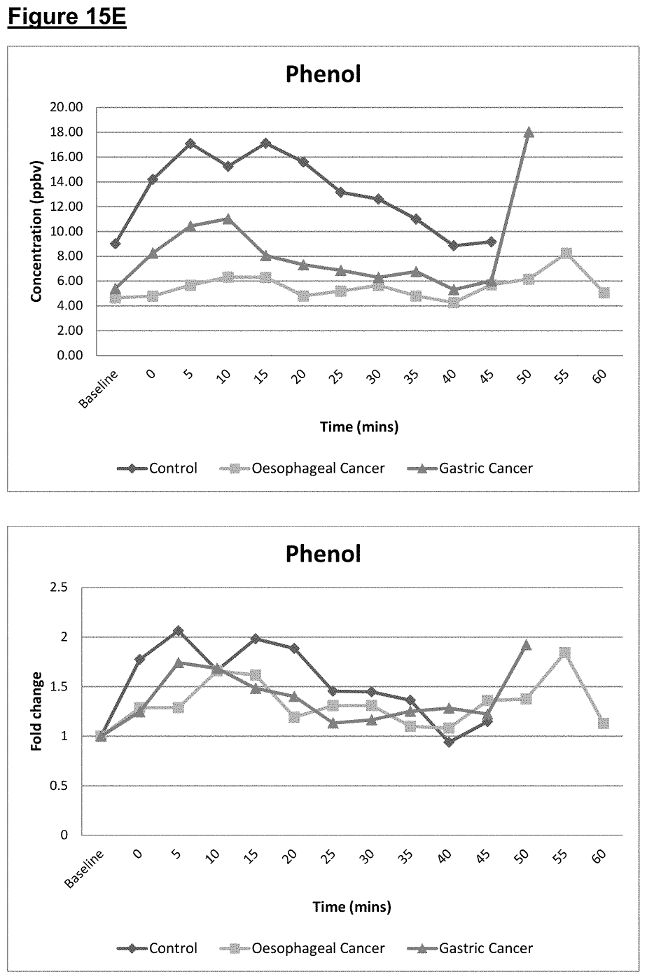

FIG. 15A to 15E shows that volatile phenols demonstrated a small increase in exhaled breath concentrations 35-45 minutes after tyrosine consumption (excluding 1-hydroxy-4-ethylbenzene);

FIG. 16 shows butanoic acid concentrations detected within exhaled breath at varying doses of four different sugars at a concentration of 25 g per 100 ml;

FIG. 17 shows decanal concentrations detected within exhaled breath with 3 g phenylalanine. Optimal response was observed at 15 minutes after phenylalanine consumption;

FIG. 18 shows dodecane concentrations detected within exhaled breath with 3 g phenylalanine. Optimal response was observed at 10 minutes after phenylalanine consumption;

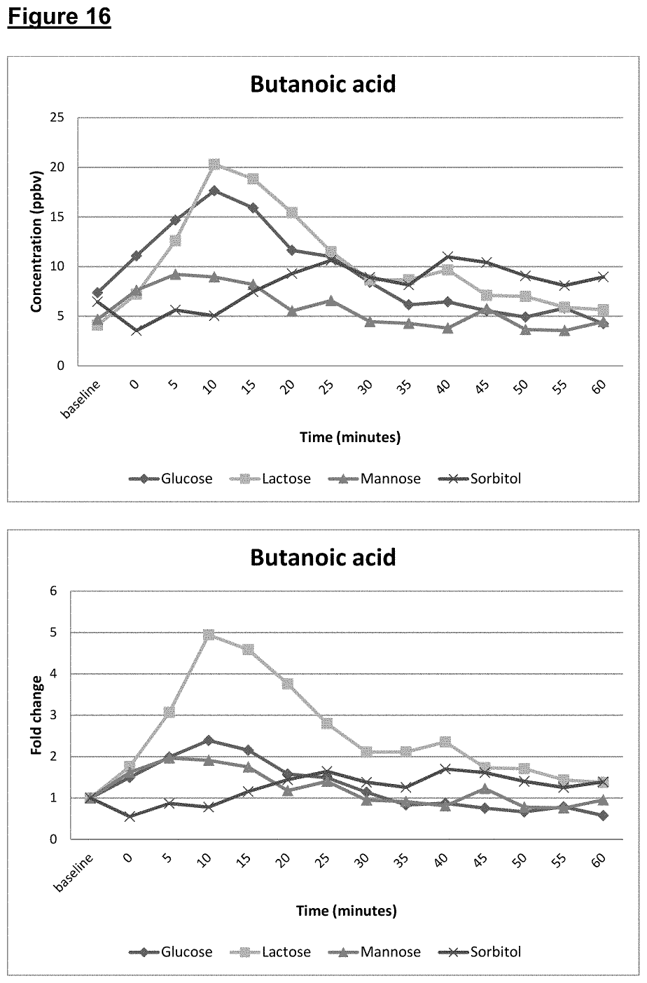

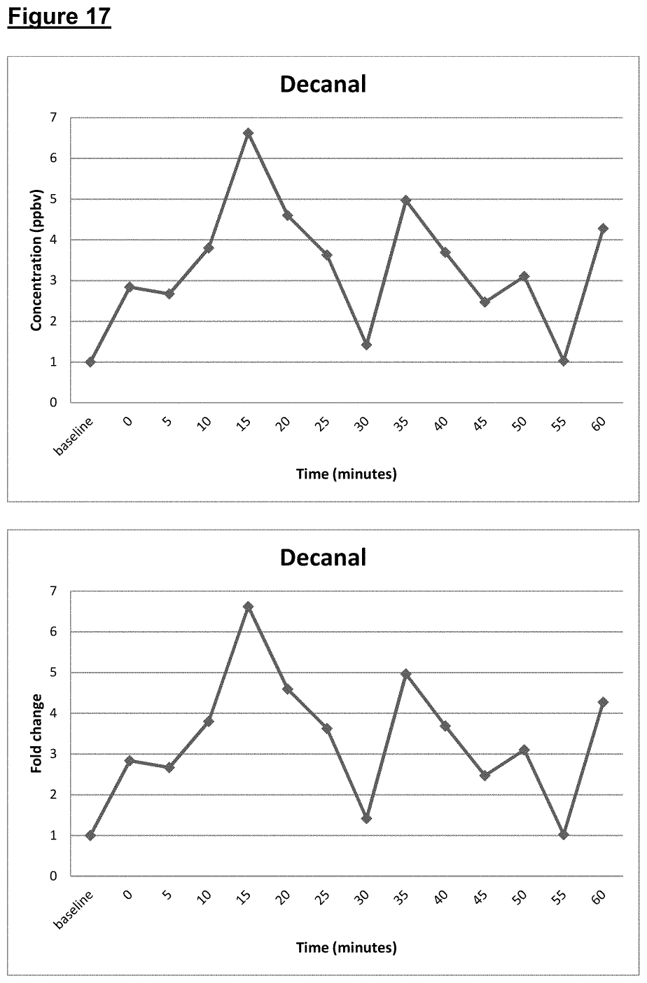

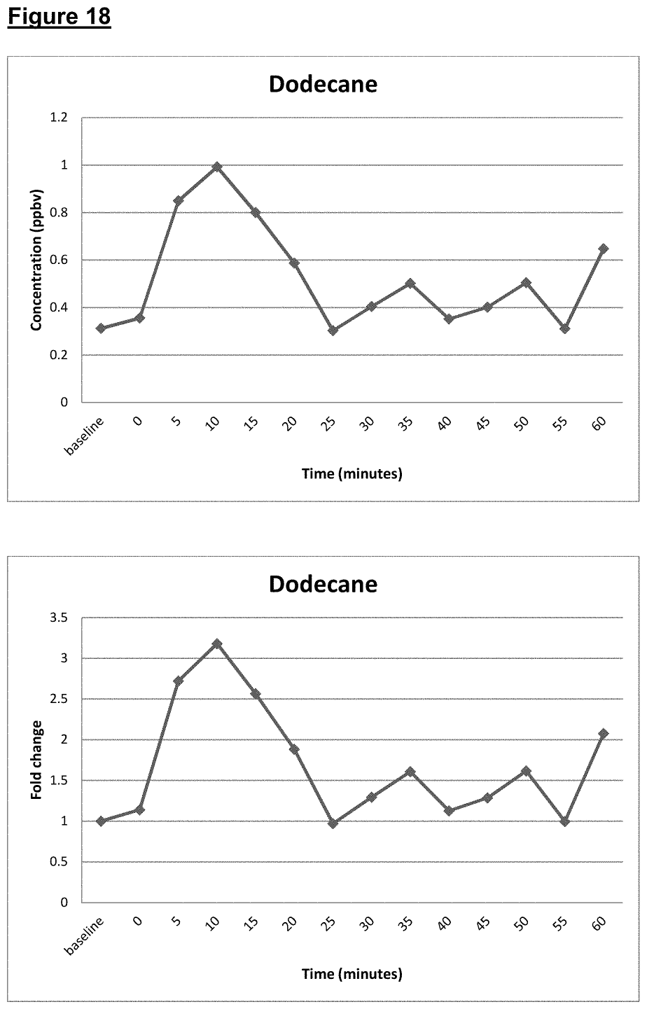

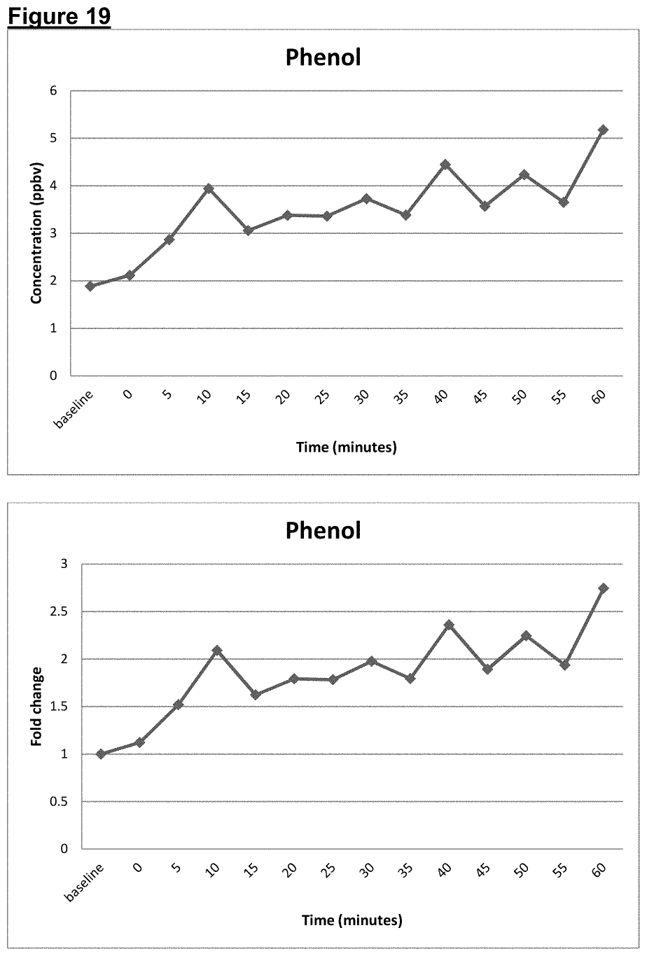

FIG. 19 shows phenol concentrations detected within exhaled breath with 3 g phenylalanine. Optimal response was observed at 60 minutes after phenylalanine consumption with 3 g phenylalanine;

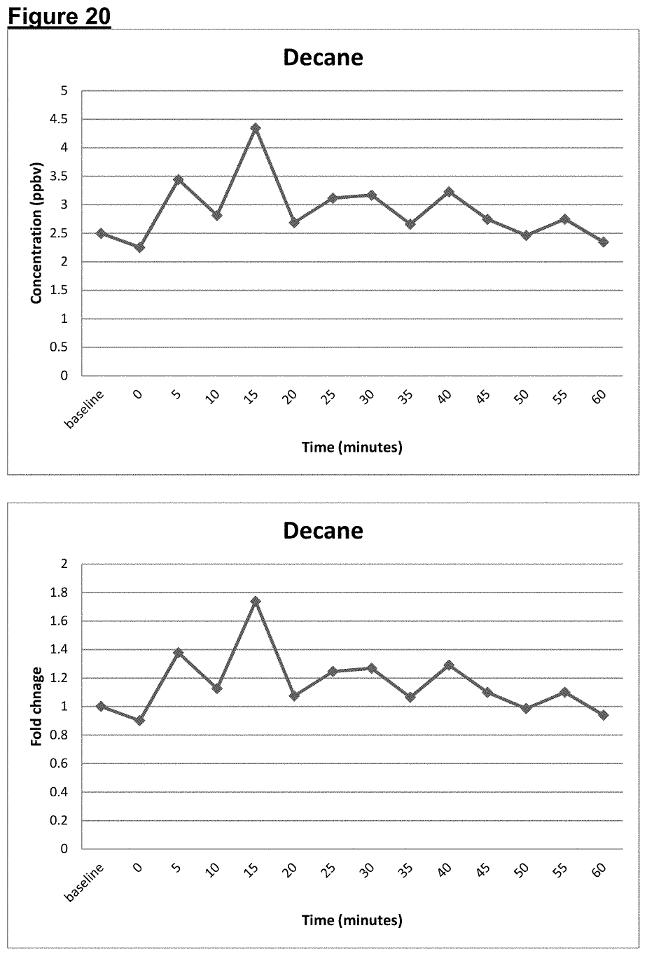

FIG. 20 shows decane concentrations detected within exhaled breath with 3 g phenylalanine. Optimal response was observed at 15 minutes after phenylalanine consumption;

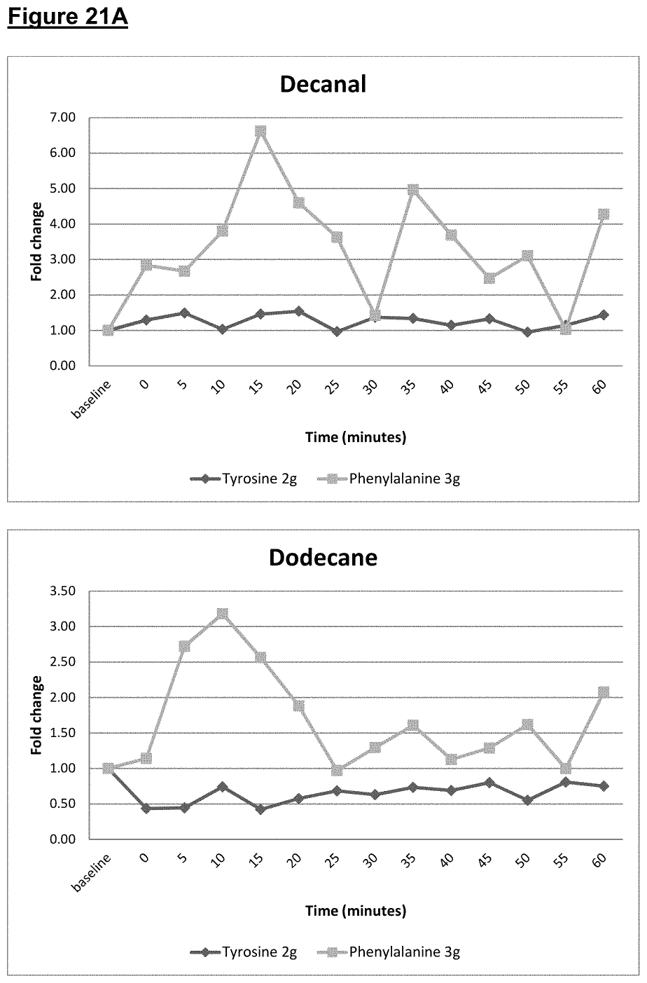

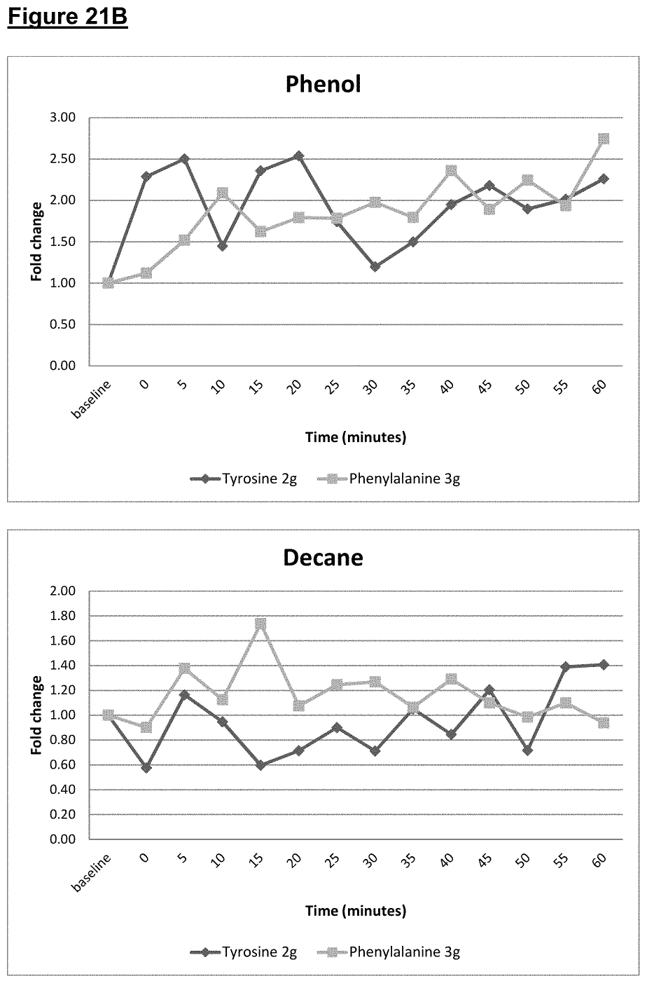

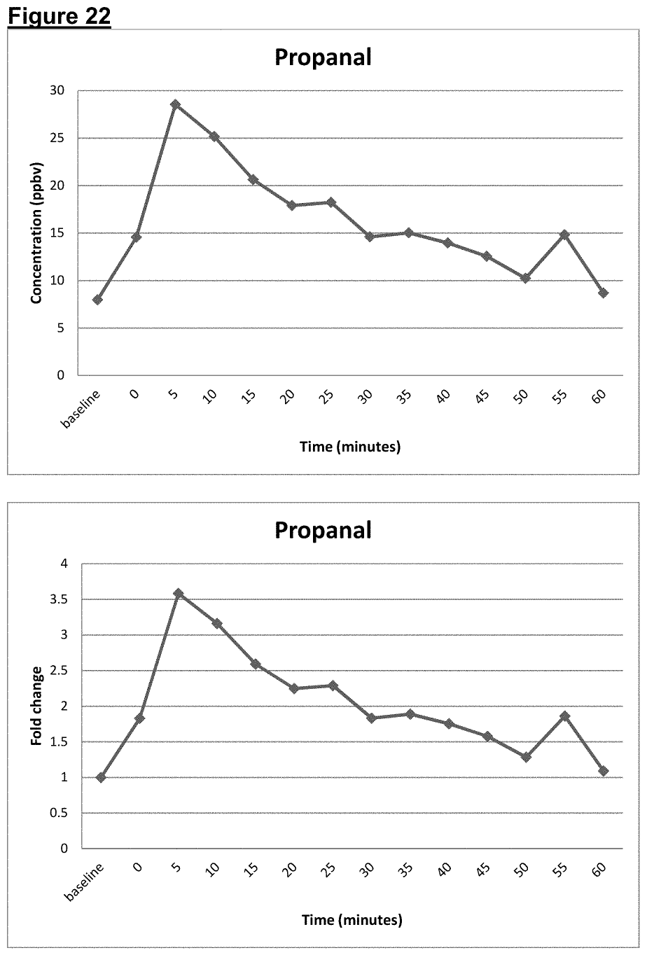

FIGS. 21A and B shows a comparison between phenylalanine and tyrosine consumption in the same subject for (a) decanal (b) dodecane (c) phenol and (d) decane. Elevated VOC responses are demonstrated with phenylalanine compared to tyrosine, most dramatically with decanal and dodecane;

FIG. 22 shows propanal concentrations detected within exhaled breath. Optimal response was observed at 5 minutes after glutamic acid consumption;

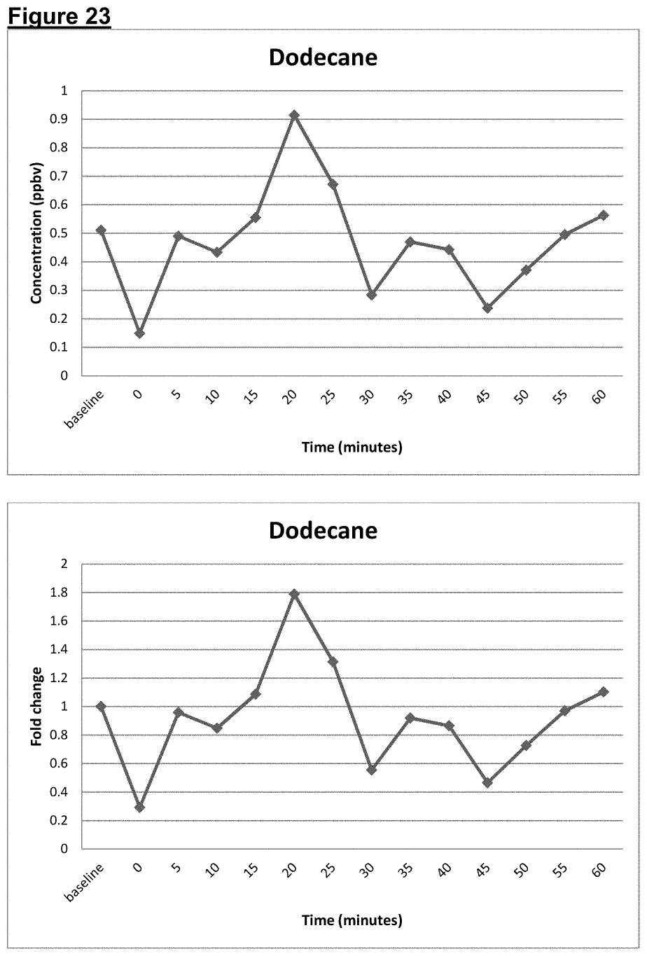

FIG. 23 shows dodecane concentrations detected within exhaled breath. Optimal response was observed at 20 minutes after glutamic acid consumption;

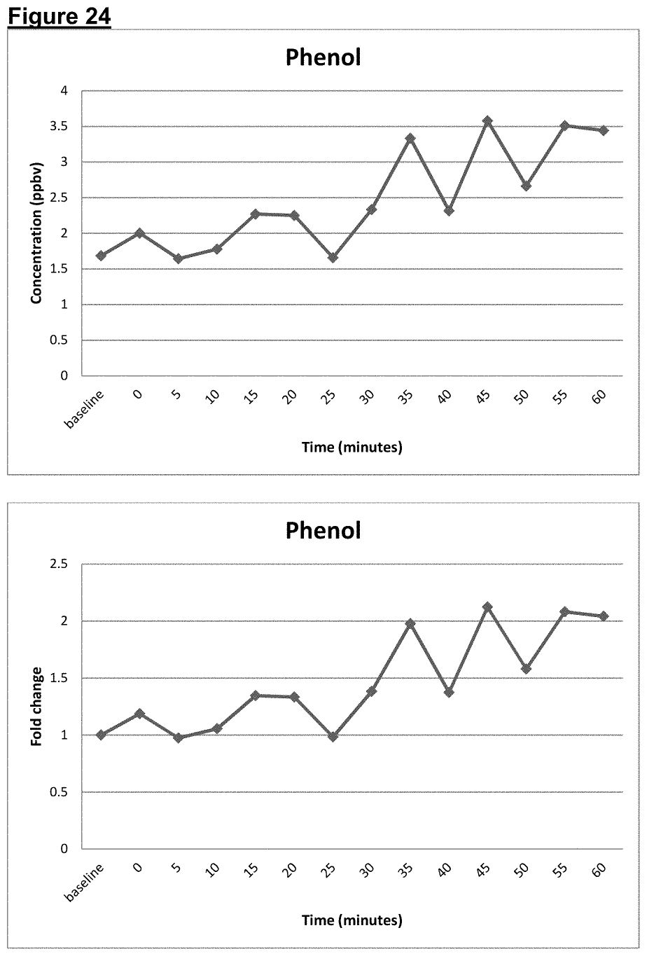

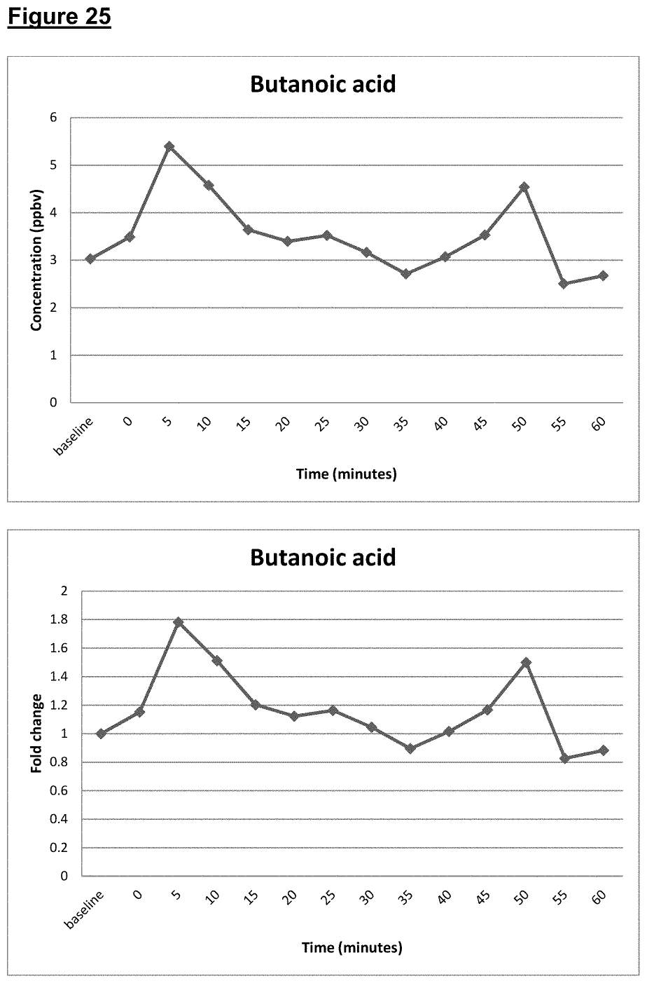

FIG. 24 shows phenol concentrations detected within exhaled breath. Optimal response was observed at 35-45 minutes after phenylalanine consumption;

FIG. 25 shows butanoic acid concentrations detected within exhaled breath. Optimal response was observed at 5 minutes after glutamic acid consumption. This is likely secondary to the production of a keto-acid during transamination of the amino acid. The keto acid is used as an intermediate in the citric acid cycle for glycolysis;

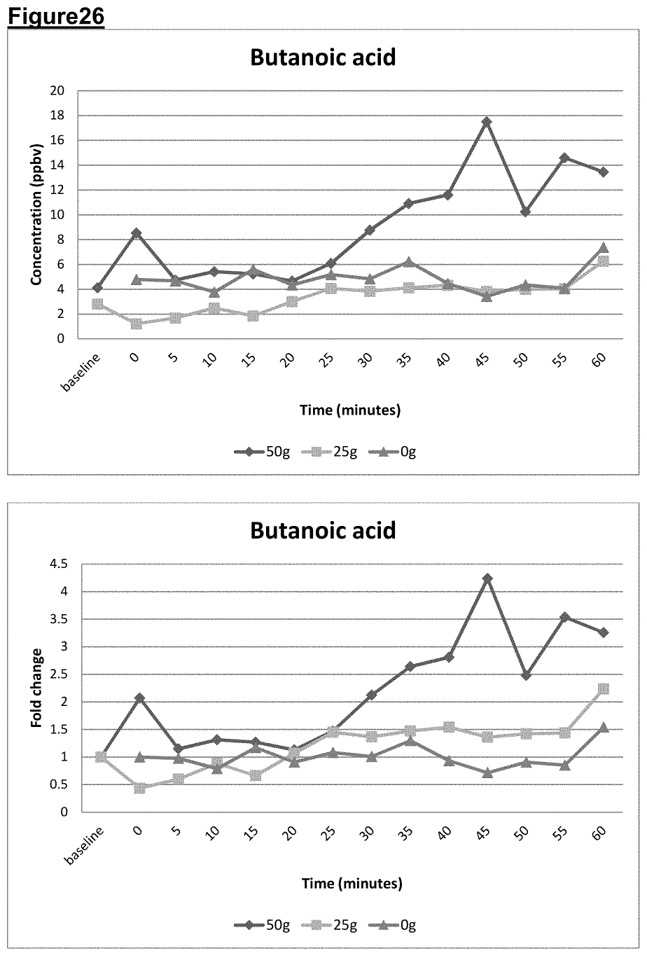

FIG. 26 shows butanoic acid concentrations detected within exhaled breath at varying doses of glycerol for subject 1. Optimal dose responses with 50 g of glycerol, 45-55 minutes after glycerol consumption;

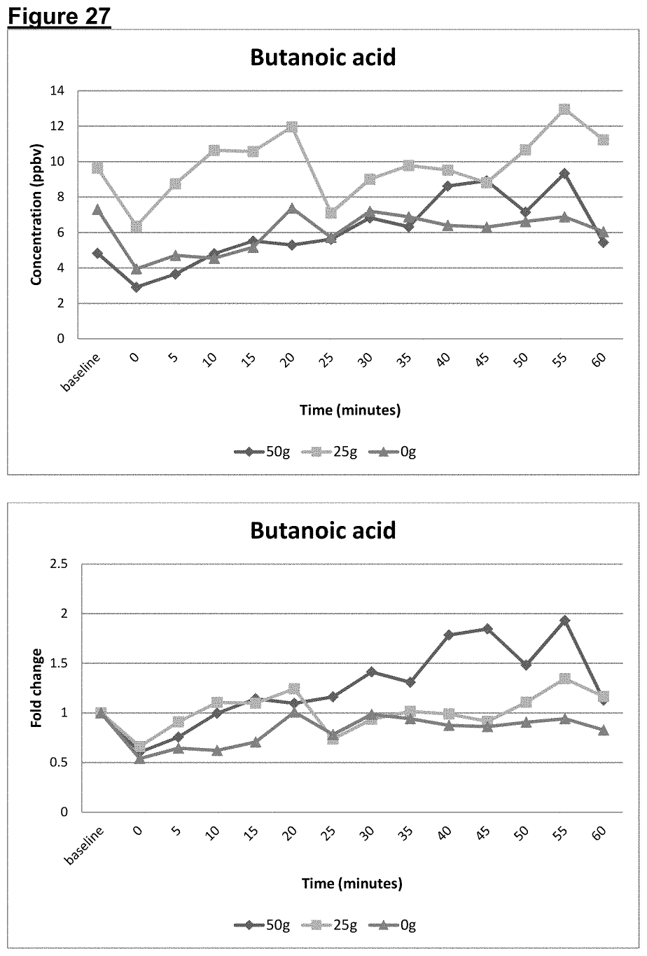

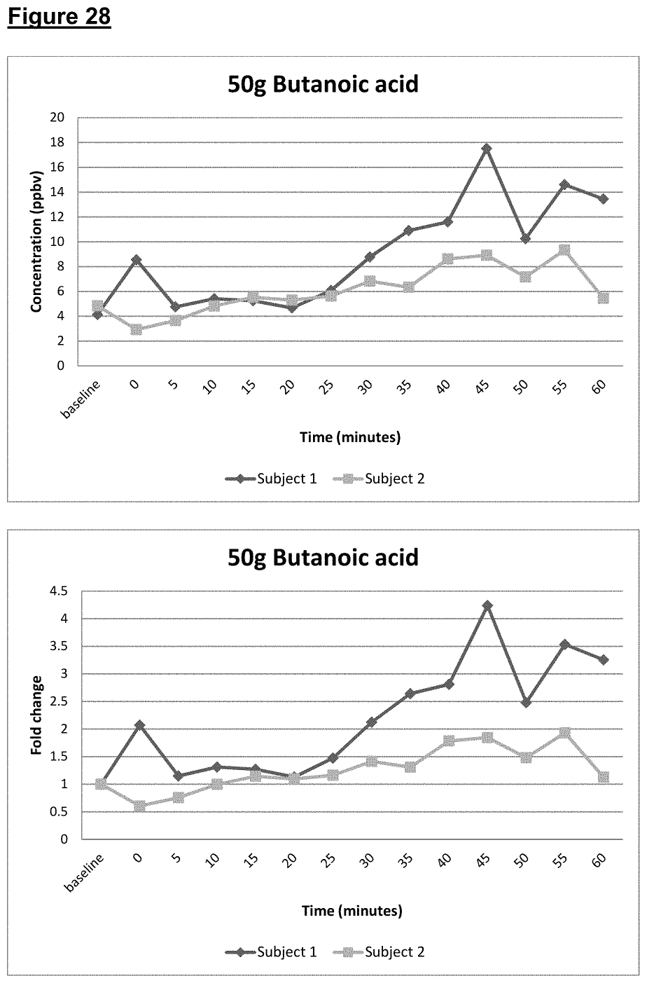

FIG. 27 shows butanoic acid concentrations detected within exhaled breath at varying doses of glycerol in subject 2. Optimal dose responses with 50 g glycerol, 45-55 minutes after glycerol consumption;

FIG. 28 shows a subject comparison between volatile butanoic acid concentrations within exhaled breath for 50 g glycerol;

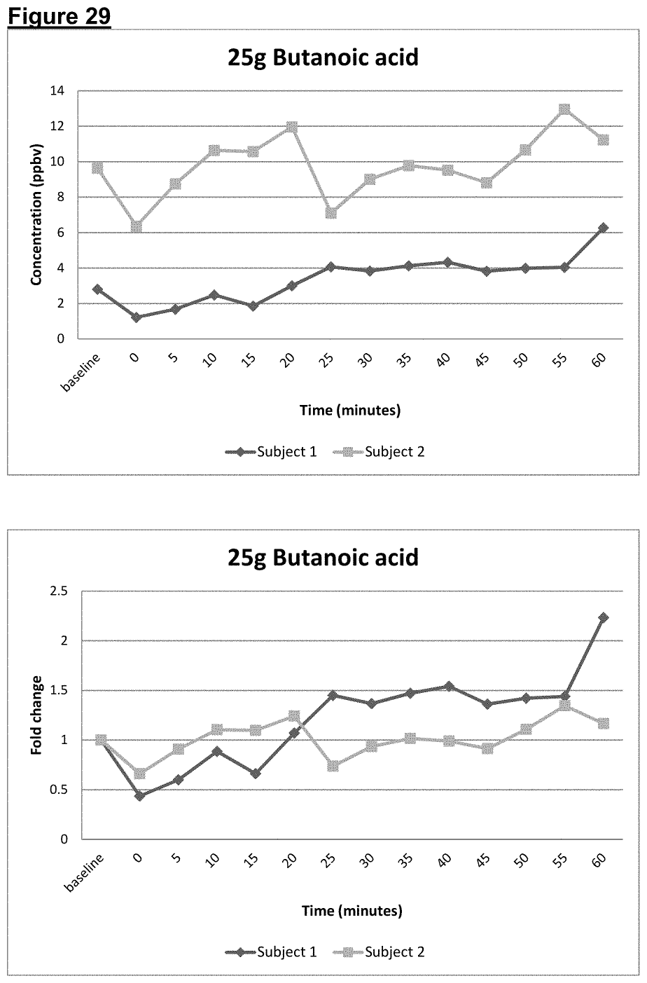

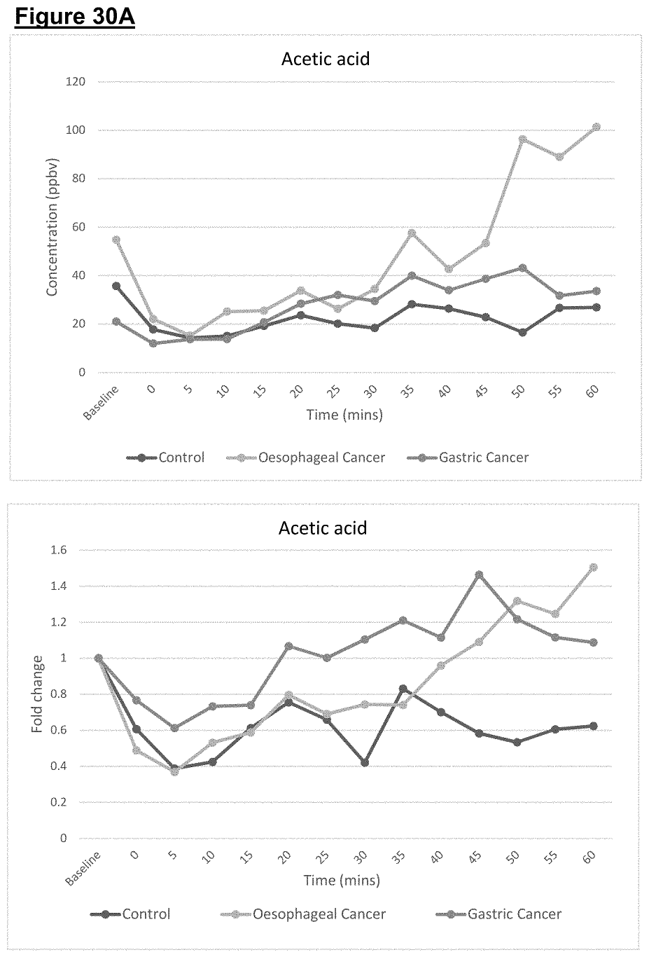

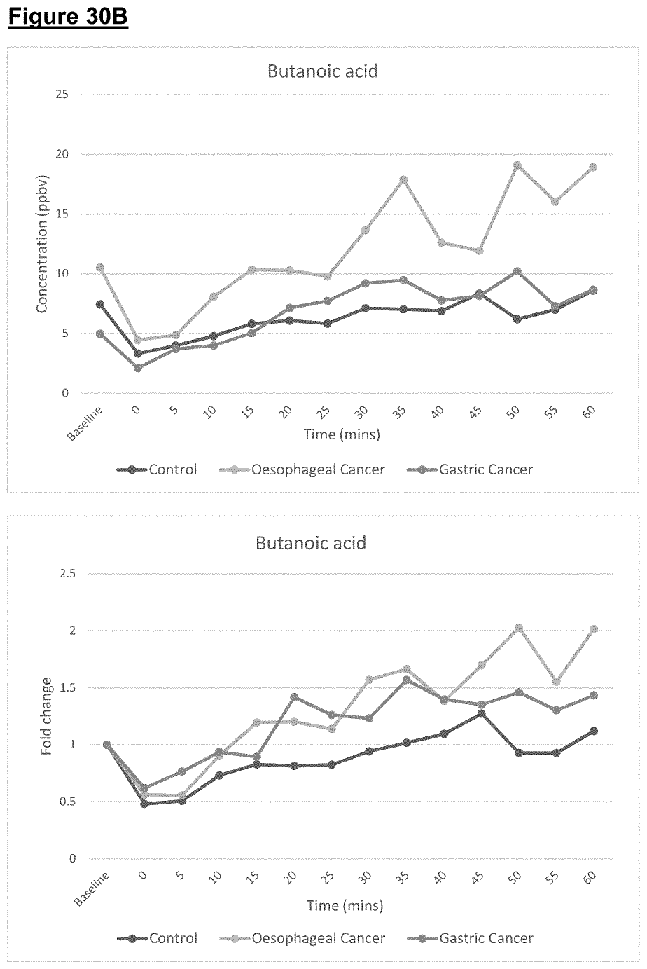

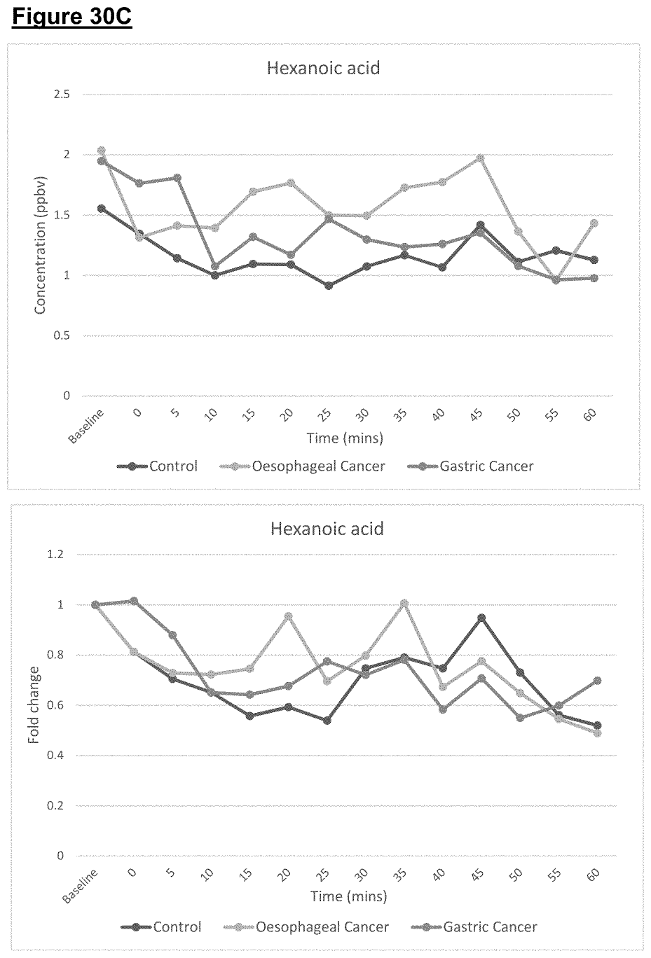

FIG. 29 shows a subject comparison between volatile butanoic acid concentrations within exhaled breath for 50 g glycerol;

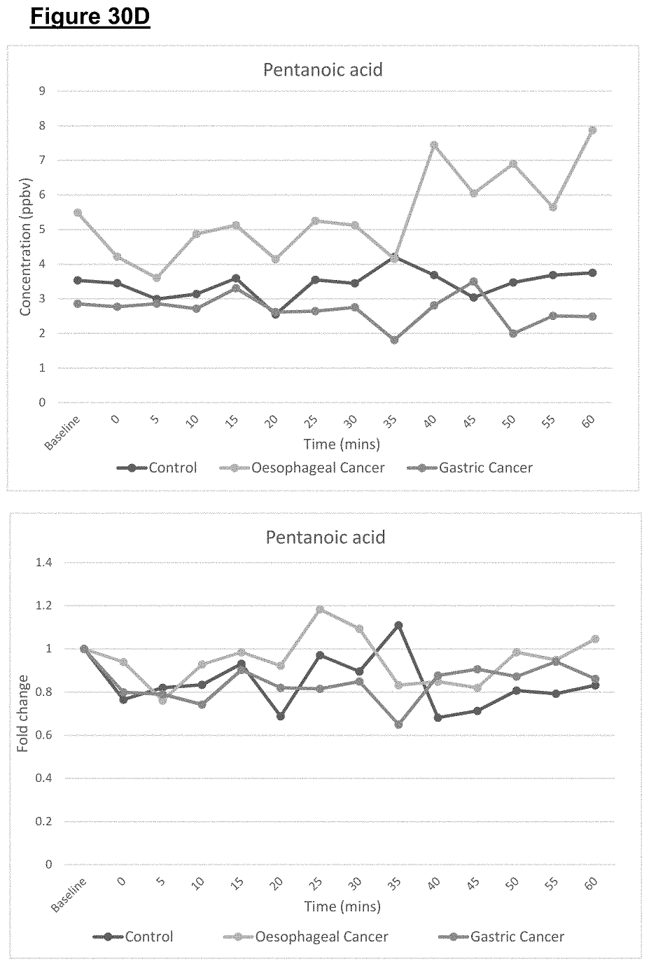

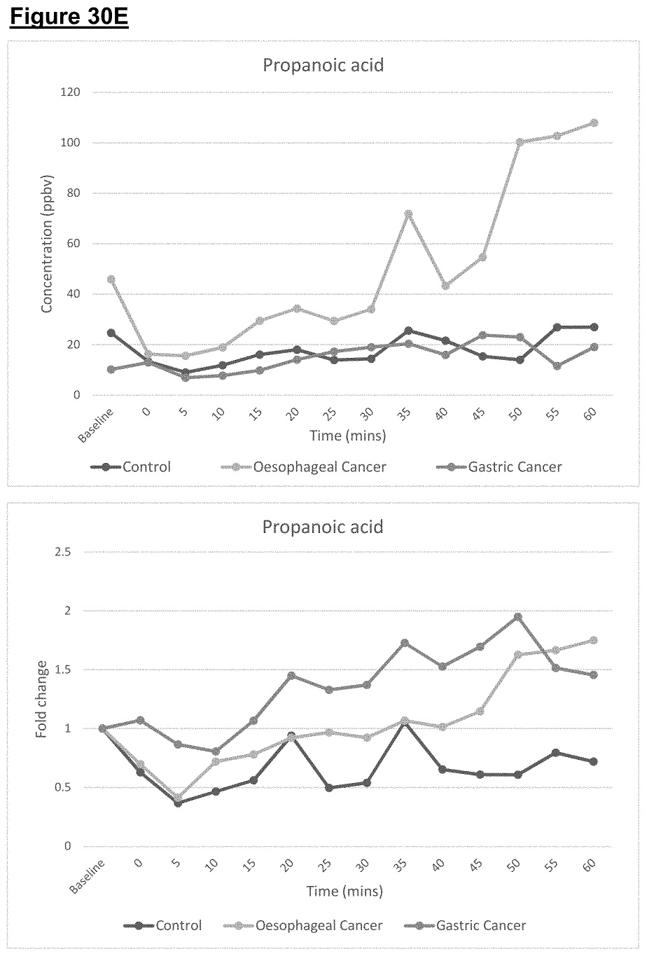

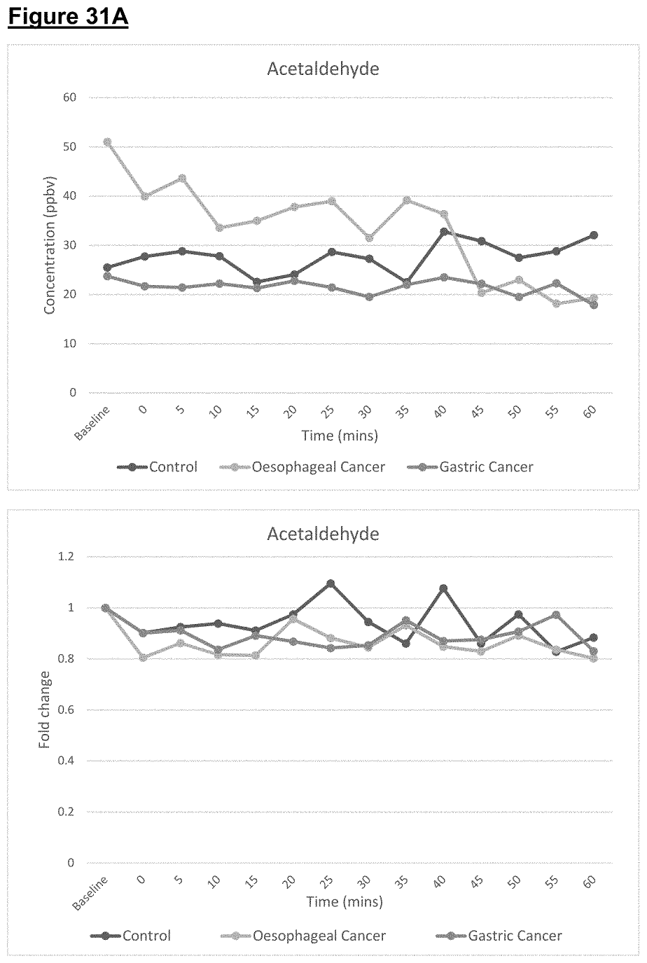

FIG. 30A to 30E shows that a number of the volatile short chain fatty acids tested (namely acetic, butanoic, and propanoic acid) increased maximally in the oesophageal cancer group at 45-60 mins after 25 g glycerol consumption;

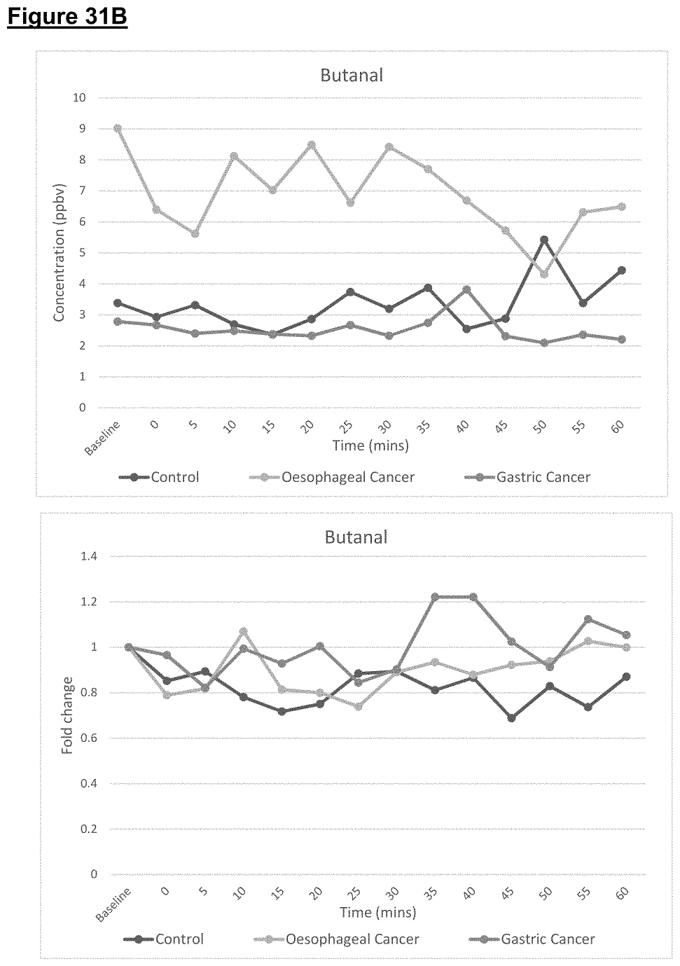

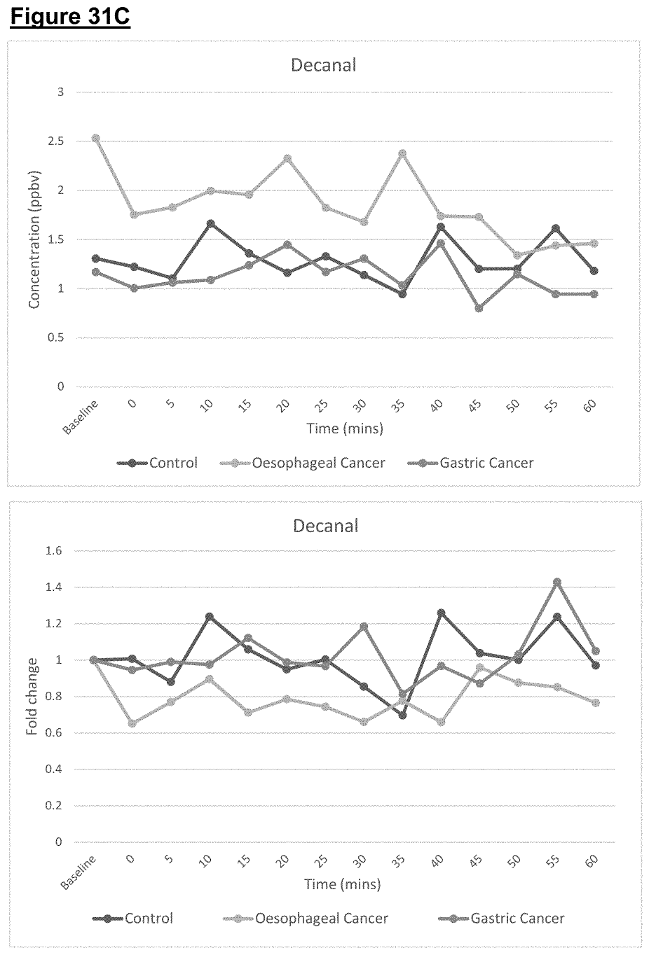

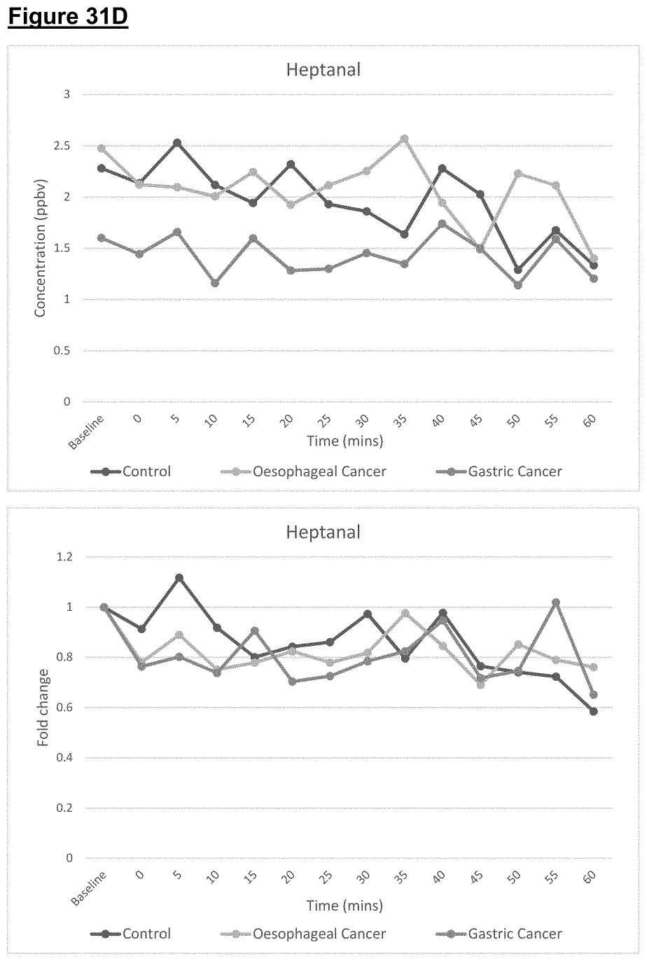

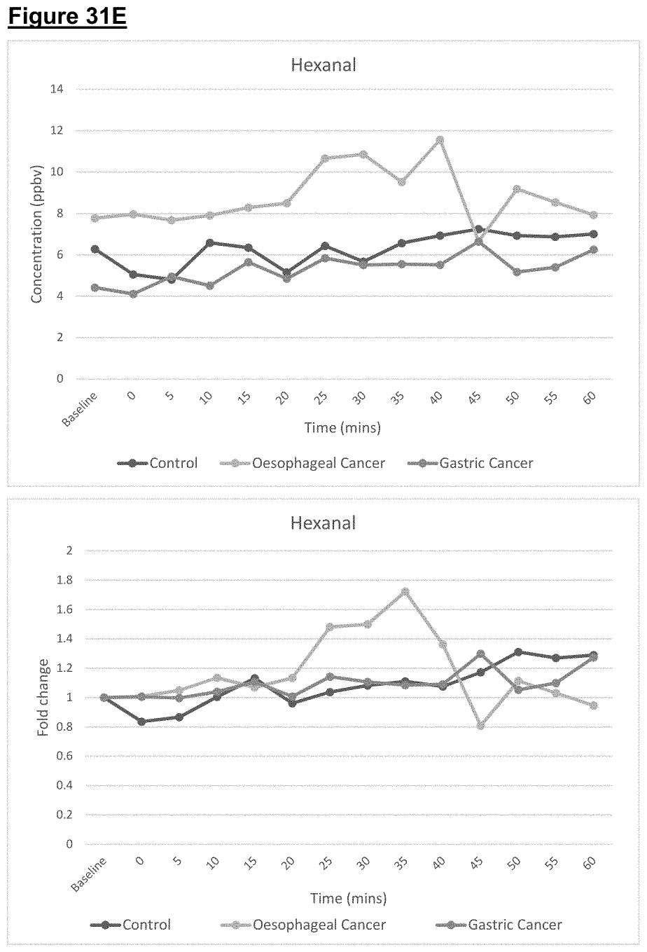

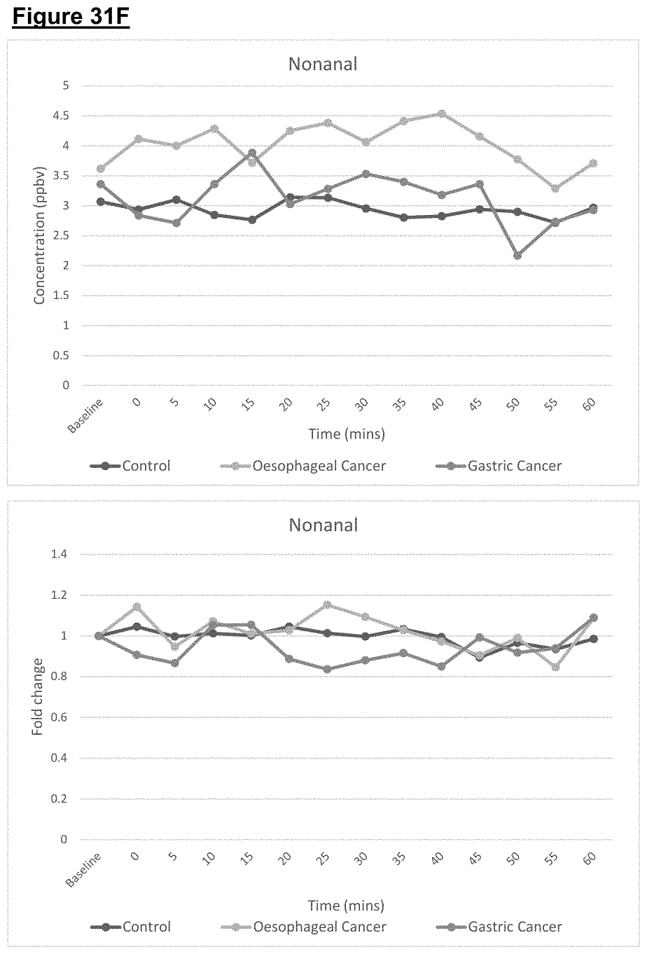

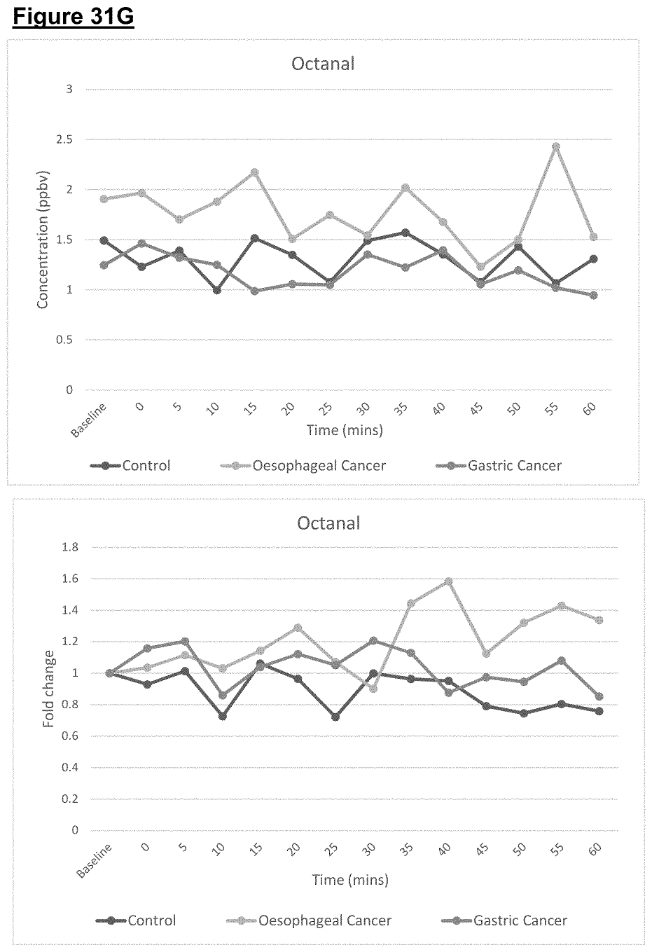

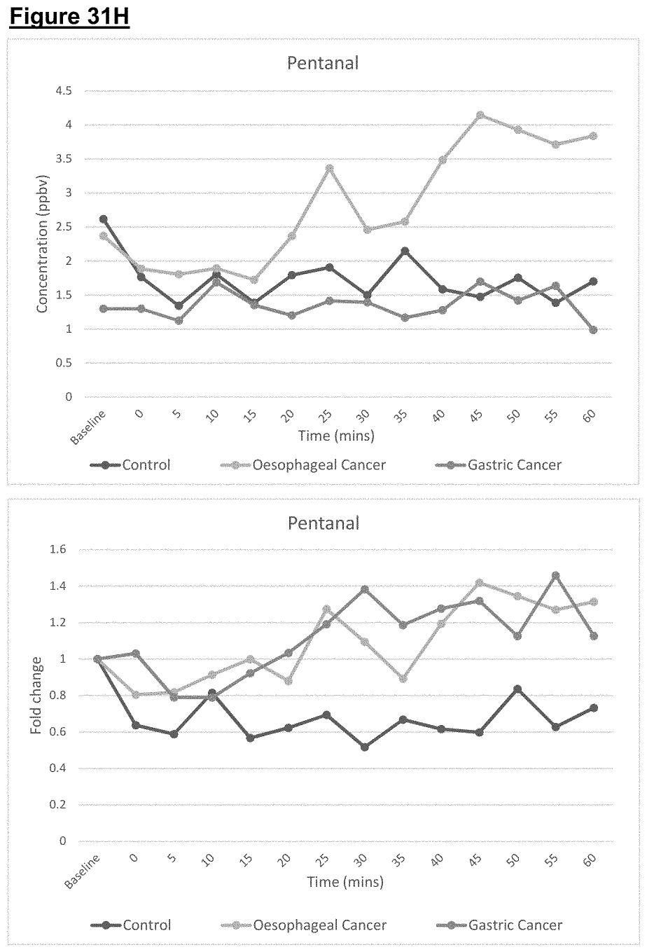

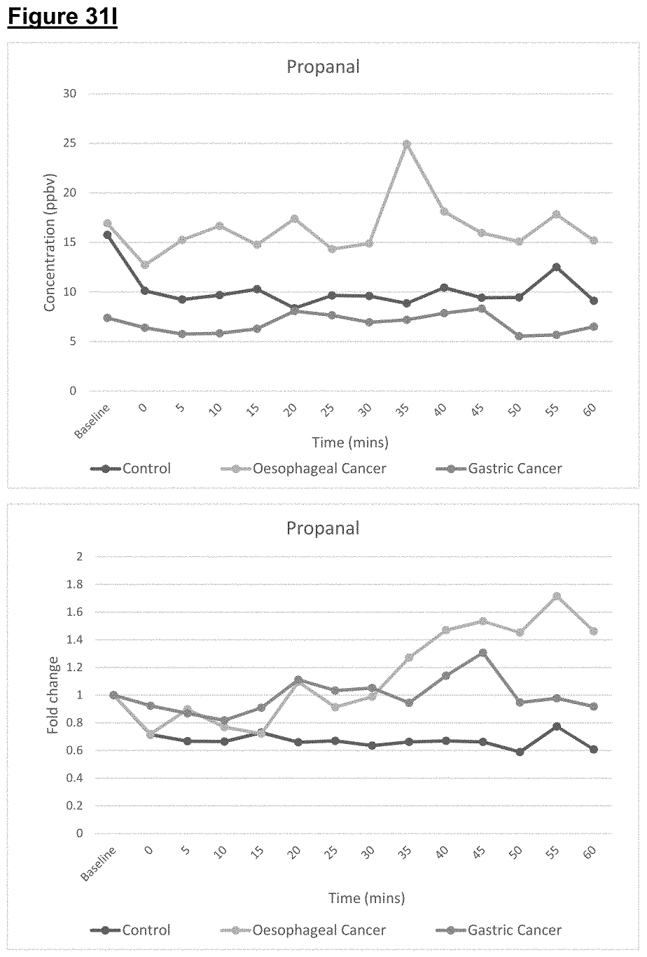

FIG. 31A to 31I shows that a number of the volatile aldehydes tested were maximally increased for the oesophageal cancer group between 40-55 minutes (hexanal, propanal, octanal and pentanal) after 25 g glycerol consumption;

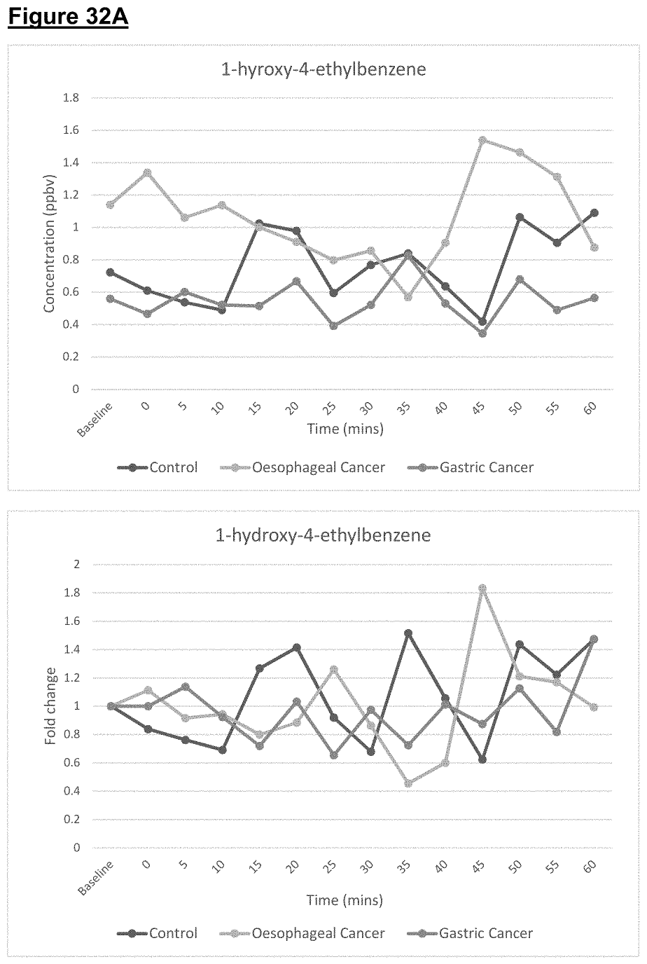

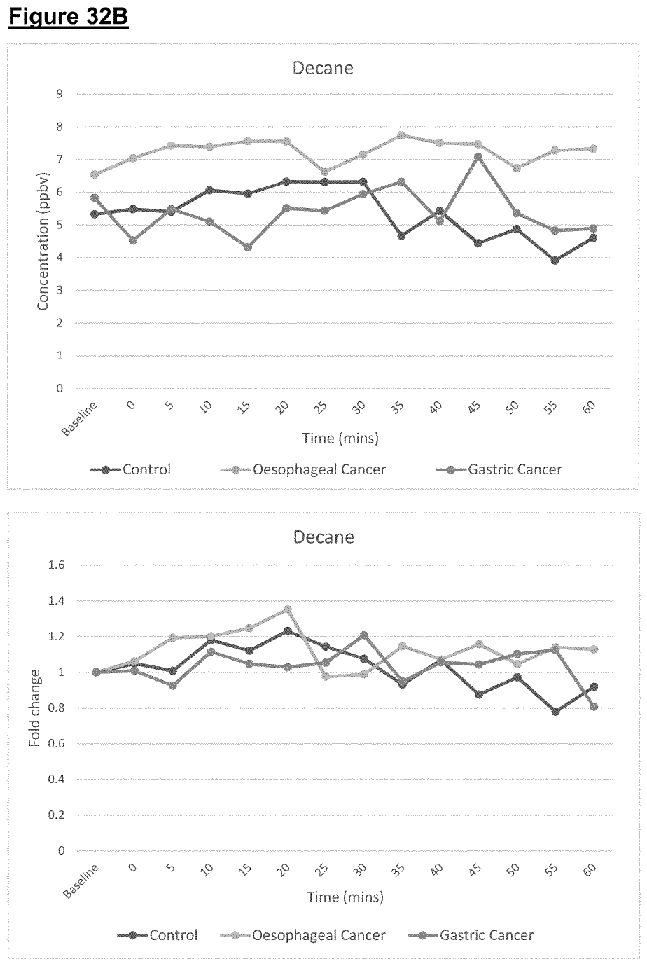

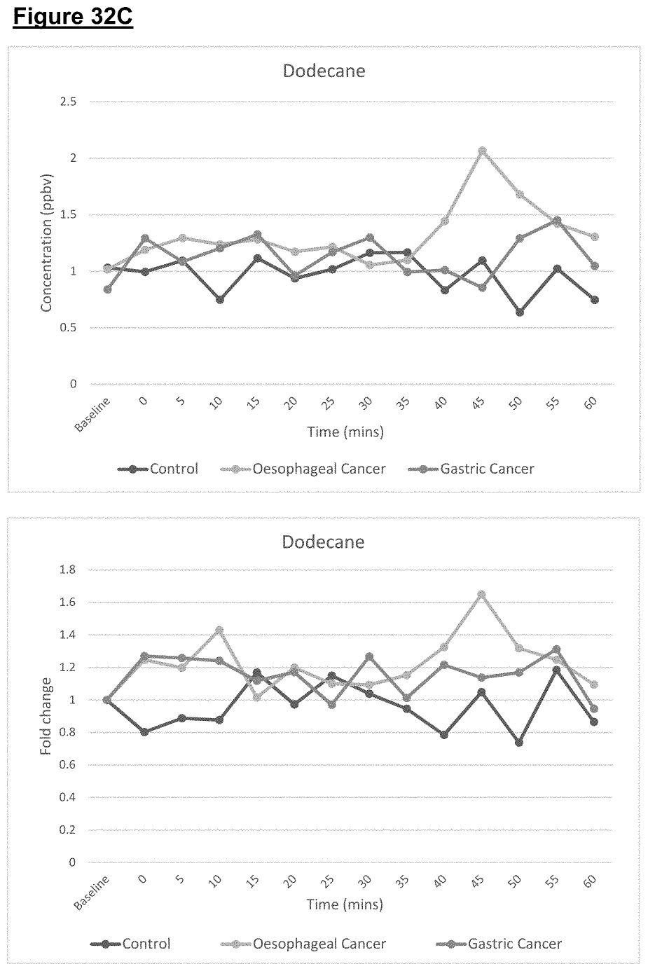

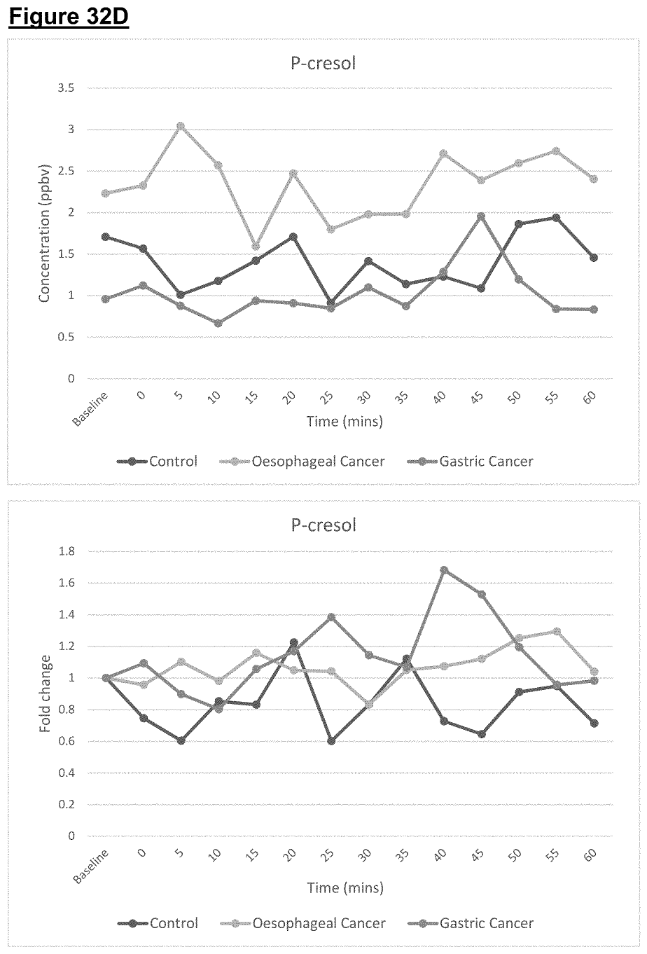

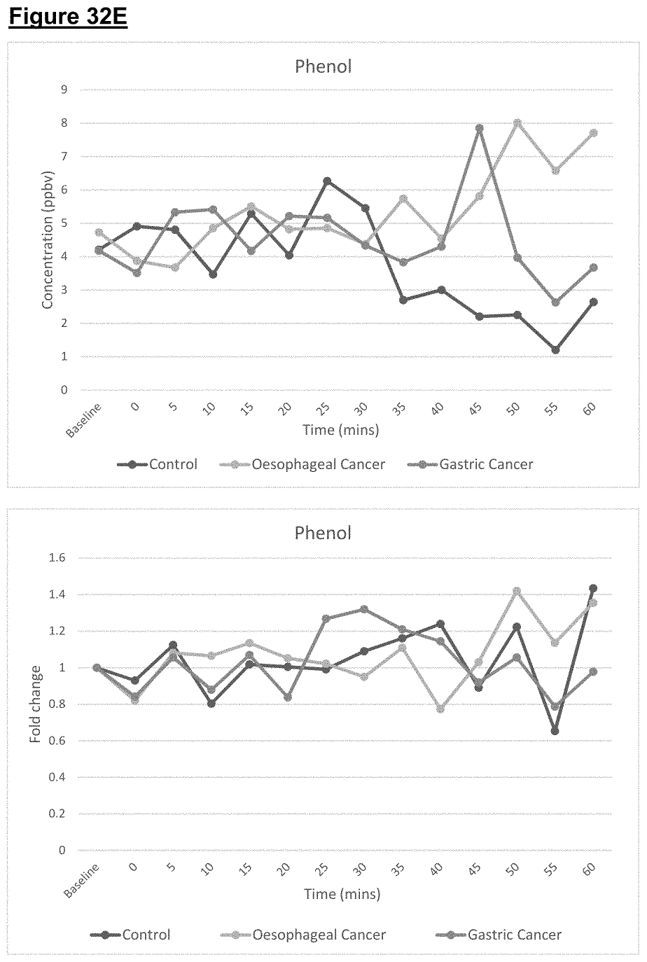

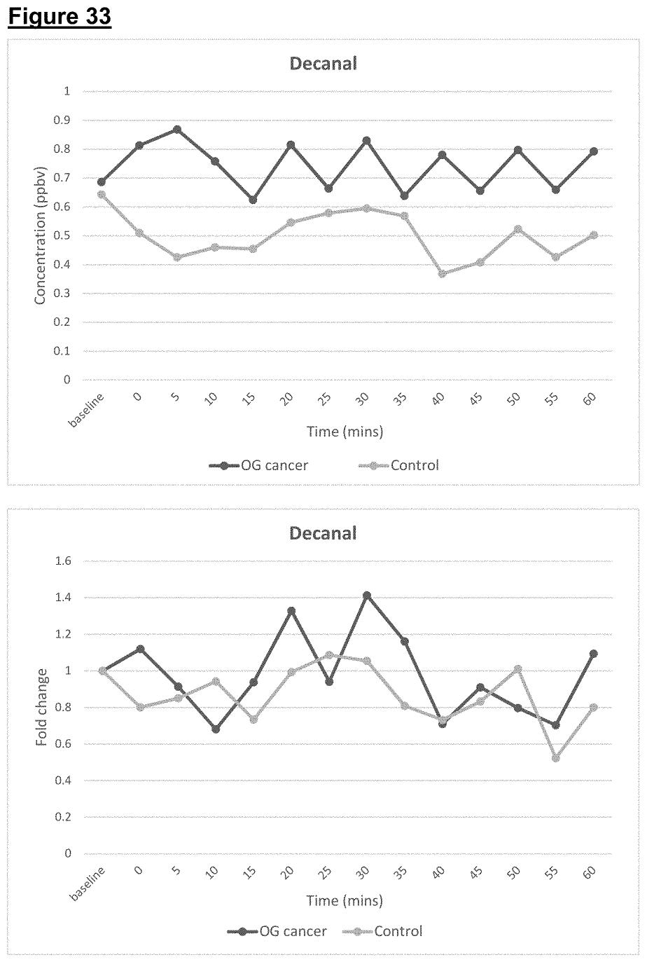

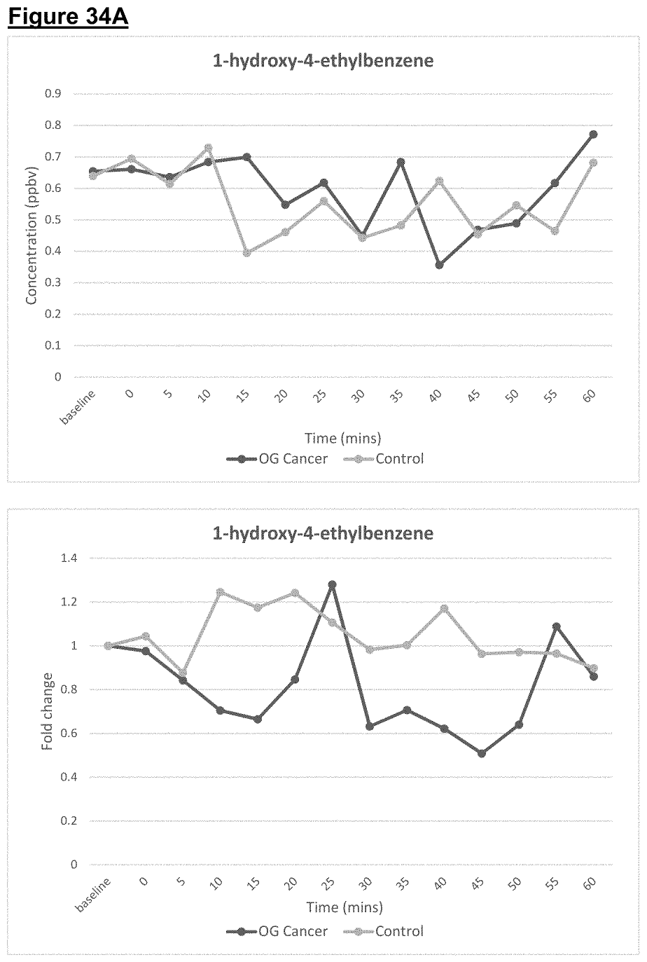

FIG. 32A to 32E shows that a number of the volatile phenols tested demonstrated no alterations in exhaled breath concentrations between the three patient groups after 25 g glycerol consumption;

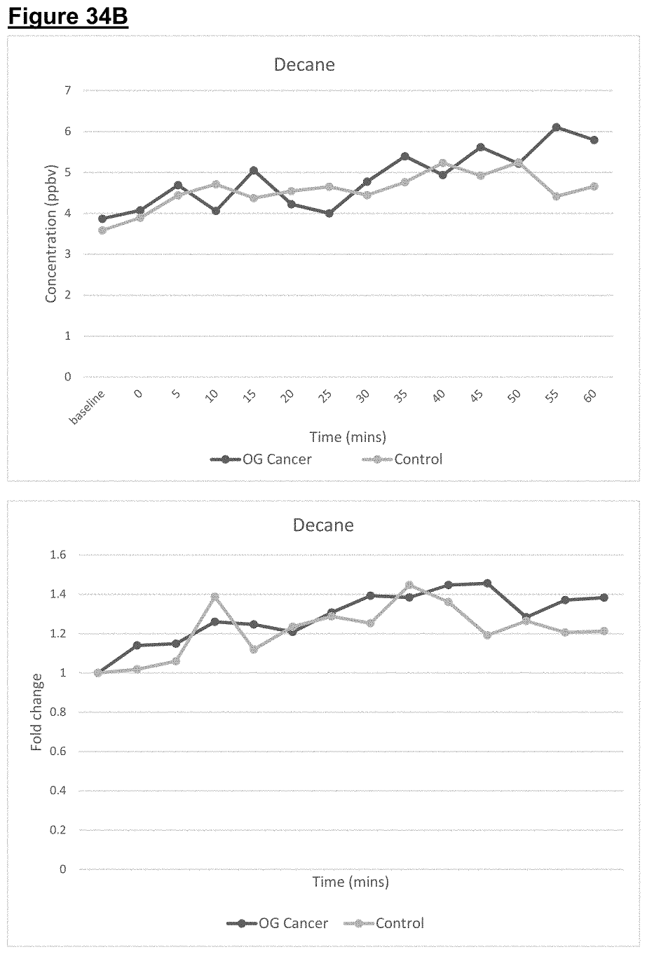

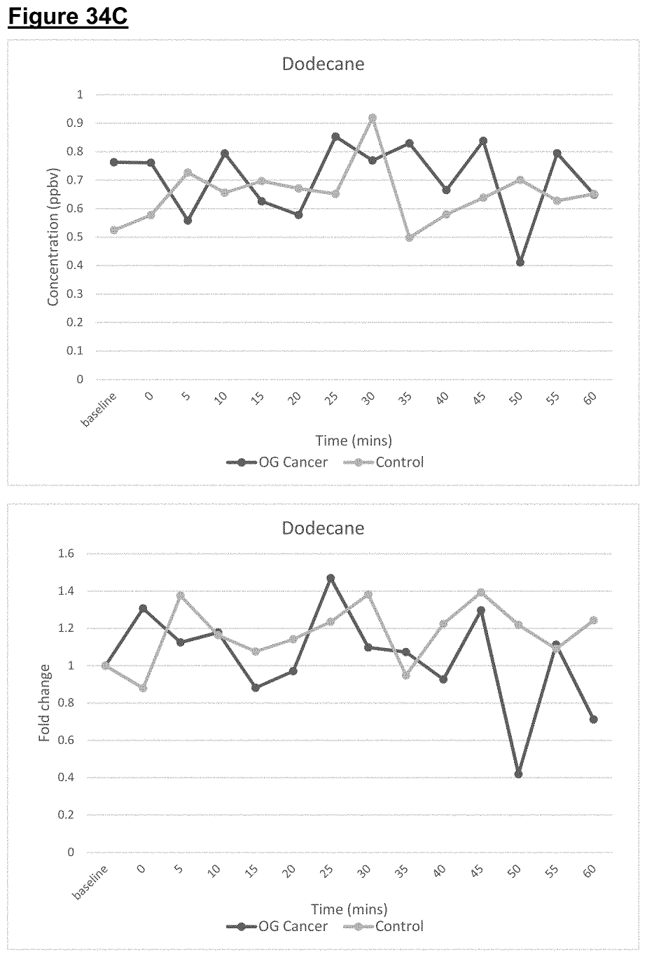

FIG. 33 shows decanal concentrations detected within exhaled breath. Optimal response was observed in the oesophagogastric cancer group at 30 minutes after consumption of the combined amino acid drink;

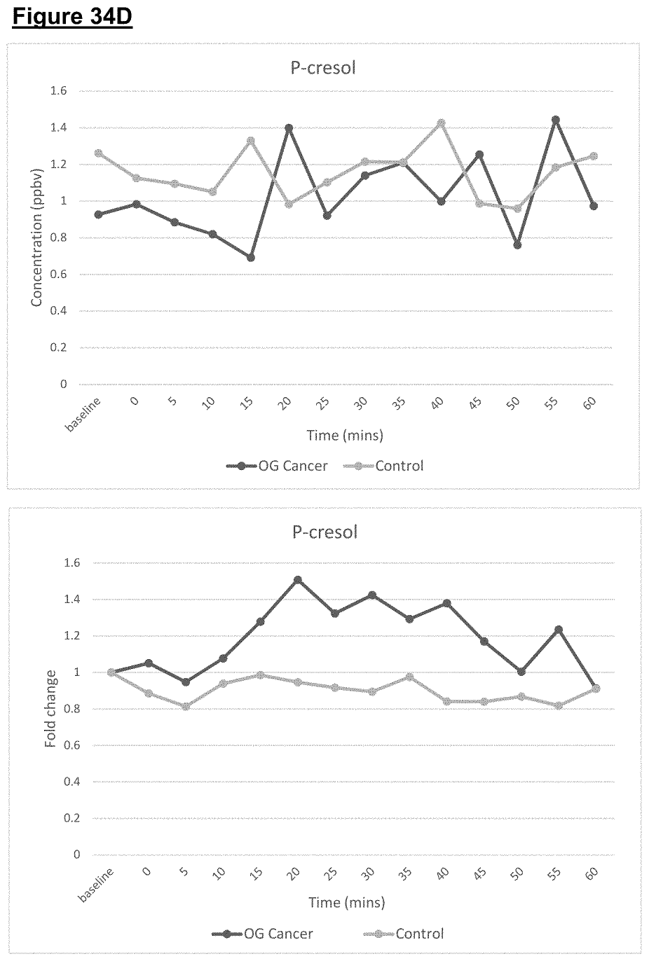

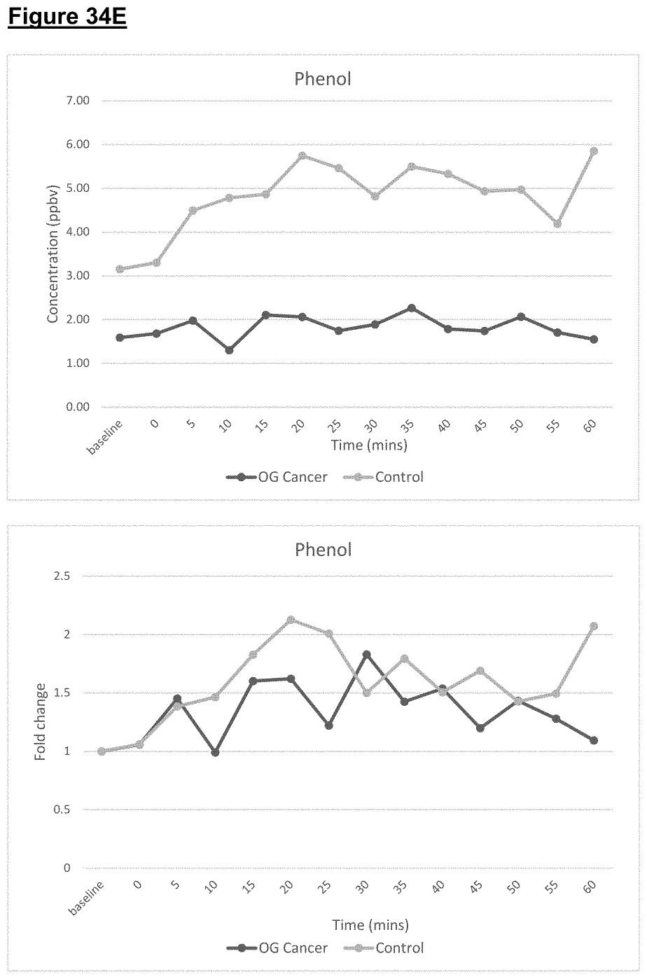

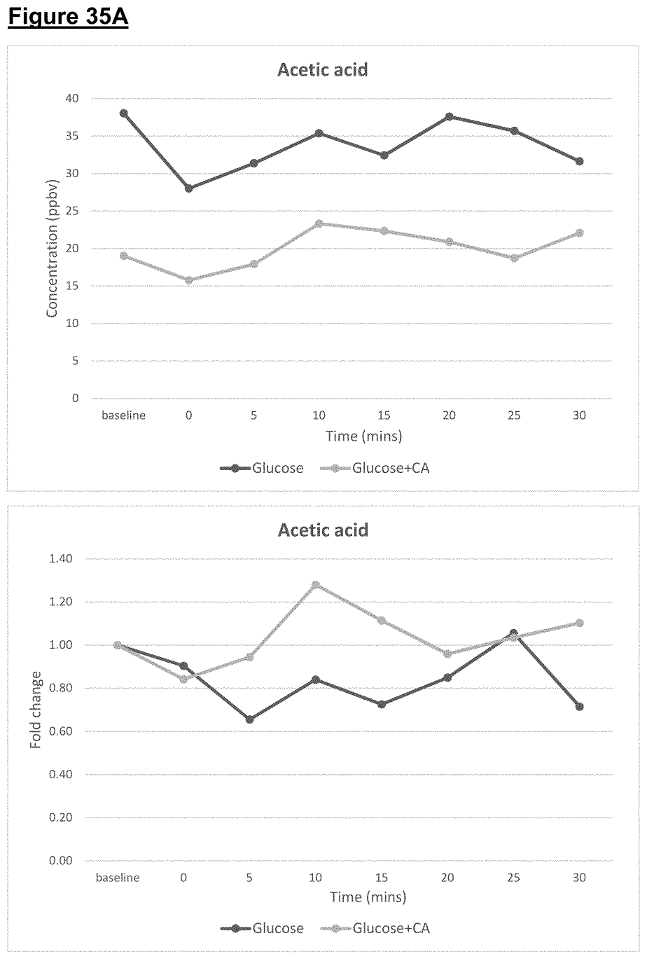

FIG. 34A to 34E shows that p-cresol was significantly increased at 40 minutes in the oesophageal cancer group after consumption of the amino acid drink. Phenol and decane showed a global increase across both cancer and non-cancer groups;

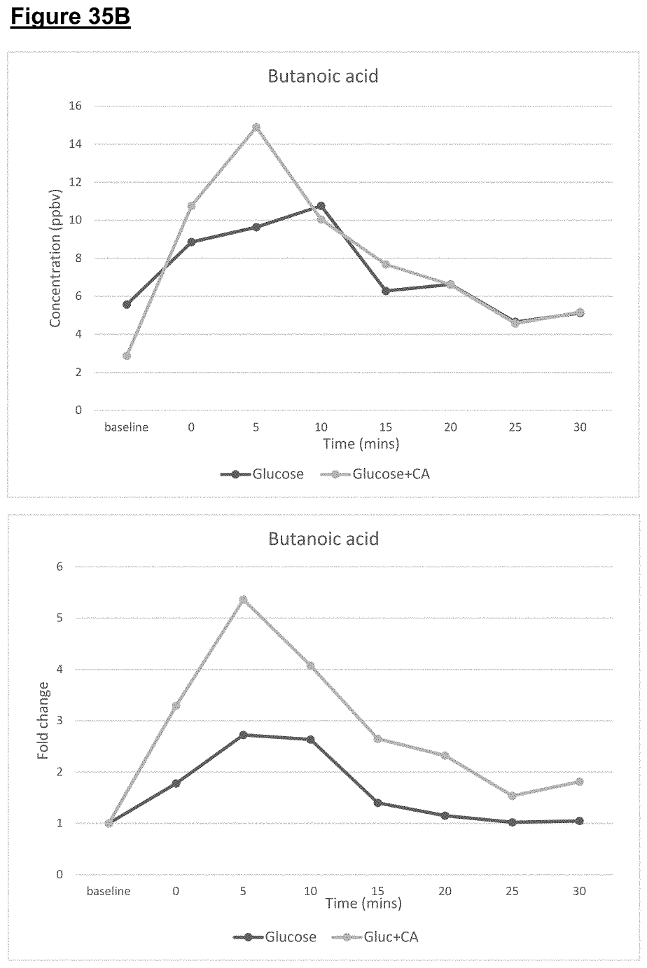

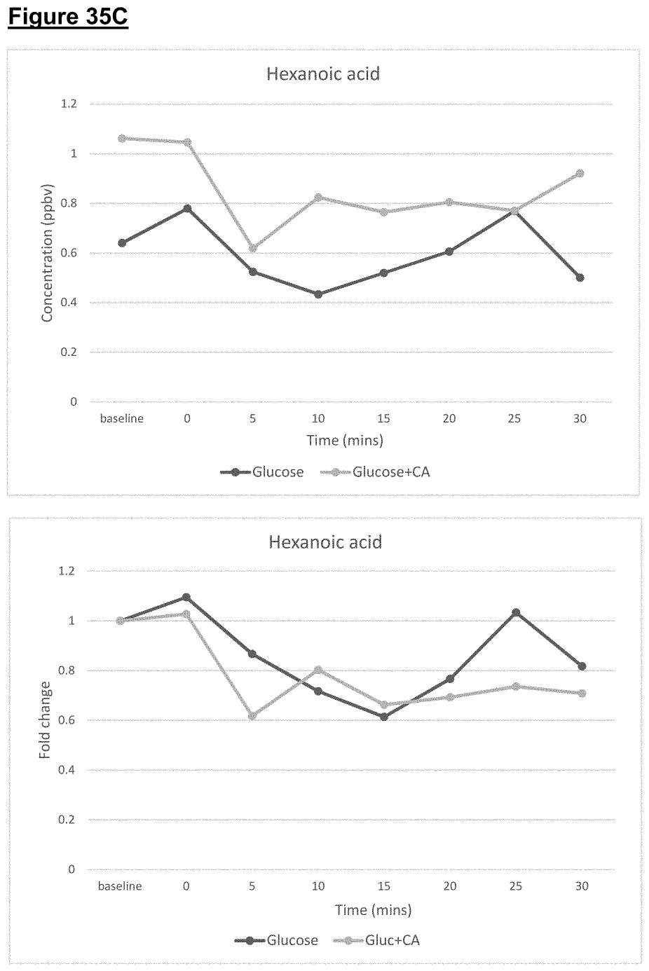

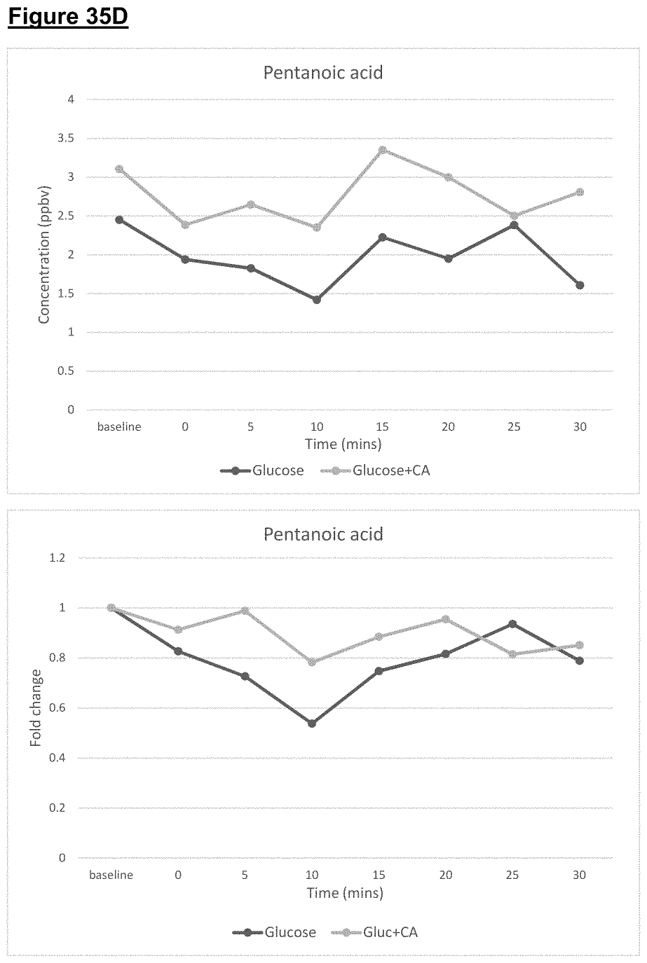

FIG. 35A to 35E shows that volatile short chain fatty acids (namely butanoic, and propanoic acid) had increased concentrations in the control group after the consumption of glucose and citric acid combined; and

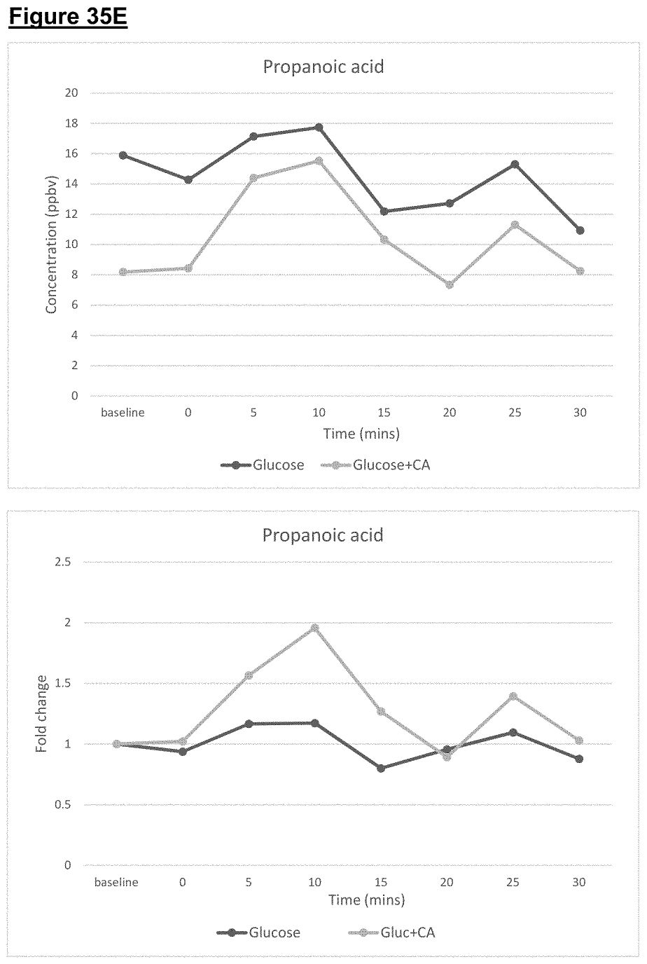

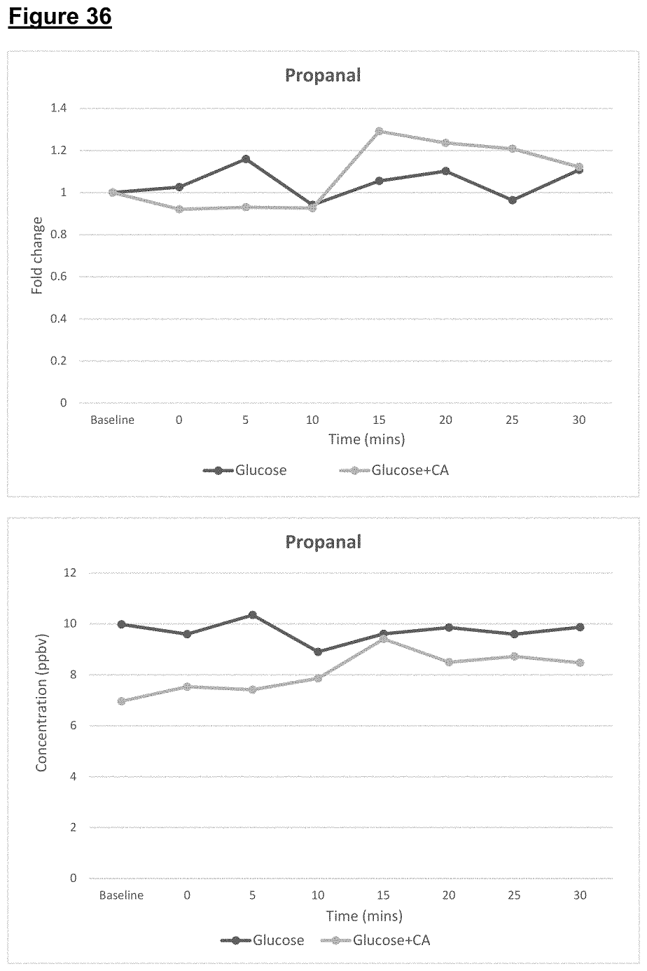

FIG. 36 shows propanal concentrations increased in the control group after consumption of glucose and citric acid combined.

### MATERIALS AND METHODS

### EXAMPLE 1

### Glucose Dose Study

Subjects

Four healthy subjects volunteered for participation and informed written consent was obtained.

Dose Concentrations

Four doses of the substrate were guided by the (i) daily recommended intake levels by the Food and Nutrition Board and (ii) the already established glucose tolerance test. The glucose tolerance test uses an acceptable 75 g of glucose dissolved in 100 ml water, which is satisfactory for patients. The daily maximum recommended dose is 130 g per day for an adult.[1] Based on these findings, the inventors selected doses of 75 g, 50 g, 25 g, 10 g to compare the dose responses against glucose concentration. All findings were compared with a baseline of 0 g.

Breath Sampling

Methods for the detection of short chain fatty acids were established on the selected ion flow tube-mass spectrometry (SIFT-MS VoiceUltra 200; Syft Technologies, Anatune, UK). All breath sampling was performed in the morning and subjects maintained a clear fluid diet for a minimum of 6 hours prior to breath sampling. All subjects exhaled directly into the inlet of the SIFT-MS using a disposable mouthpiece. A baseline breath test was performed for each method followed by consumption of glucose dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C. Data was obtained in concentrations in parts per billion.

Sugars

Comparison of 4 different sugars at a dose of 25 g each. 25 g was chosen after the initial glucose study where similar VOC concentrations were observed between 25 g and 75 g. Glucose, lactose and mannose follow a similar pattern with an increase maximally at 10 minutes after sugar consumption (FIG. 16). Lactose is a disaccharide composed of both glucose and galactose, and without wishing to be bound to any particular theory is expected to follow a similar pattern to glucose. Similarly, mannose is a simple sugar, also known to be an isomer of glucose and is without wishing to be bound to any particular theory is thought to be metabolised via the same glycolytic pathway.

Glucose

Patient Selection

All patients were recruited from St Mary's Hospital from February 2019-May 2019. Patients were recruited from three cohorts; oesophagial cancer (n=6), gastric cancer (n=6) and age-matched healthy controls (n=6). Informed written consent was obtained by all participants. Patients diagnosed with oesophagogastric adenocarcinoma ranged from early disease on the curative pathway to metastatic palliative disease. Age-matched healthy controls included patients with benign upper gastrointestinal disease (reflux, dysmotility) or healthy asymptomatic controls. Demographic and clinical information was collated.

Breath Sampling

Methods for the detection of 4 classes of volatile compounds; short chain fatty acids, alcohols, aldehydes and phenol-alkanes were established on the selected ion flow tube-mass spectrometry (SIFT-MS VoiceUltra 200; Syft Technologies, Anatune, UK). All breath sampling was performed in the morning and patients maintained a clear fluid diet for a minimum of 6 hours prior to breath sampling. All patients exhaled directly into the inlet of the SIFT-MS using a disposable mouthpiece. A baseline breath test was performed for each method followed by consumption of 25 g glucose dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C.

Statistical Analysis

Data was obtained in concentrations in parts per billion. Univariate Kruskal Wallis analysis was performed across the three groups using SPSS statistical software (v25, Armonk N.Y.; IBM Corp). Mann Whitney U test was performed to identify differences between oesophageal and gastric cancers compared with controls. P value of <0.05 was considered statistically significant.

### EXAMPLE 2

### Tyrosine

Patient Selection

All patients were recruited from St Mary's Hospital from February 2019-May 2019. Patients were recruited from three cohorts; oesophageal cancer (n=6), gastric cancer (n=6) and age-matched healthy controls (n=6). Informed written consent was obtained by all participants. Patients diagnosed with oesophagogastric adenocarcinoma ranged from early disease on the curative pathway to metastatic palliative disease. Age-matched healthy controls included patients with benign upper gastrointestinal disease (reflux, dysmotility) or healthy asymptomatic controls. Demographic and clinical information was collated.

Breath Sampling

Methods for the detection of 4 classes of volatile compounds; short chain fatty acids, alcohols, aldehydes and phenol-alkanes were established on the selected ion flow tube-mass spectrometry (SIFT-MS VoiceUltra 200; Syft Technologies, Anatune, UK). All breath sampling was performed in the morning and patients maintained a clear fluid diet for a minimum of 6 hours prior to breath sampling. All patients exhaled directly into the inlet of the SIFT-MS using a disposable mouthpiece. A baseline breath test was performed for each method followed by consumption of 2 g tyrosine dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C.

Statistical Analysis

Data was obtained in concentrations in parts per billion. Univariate Kruskal Wallis analysis was performed across the three groups using SPSS statistical software (v25, Armonk N.Y.; IBM Corp). Mann Whitney U test was performed to identify differences between oesophageal and gastric cancers compared with controls. P value of <0.05 was considered statistically significant.

### EXAMPLE 3

### Phenylalanine

Subjects: One healthy subject.

Dose concentrations: The daily recommended intake levels advised by the Food and Nutrition Board is 100 mg/kg daily for an adult, with a maximum dose of 3 g. [1] A single dose of 3 g was selected for this study.

Breath Sampling

Methods for the detection of short chain fatty acids, aldehydes and phenol-alkanes were established on the selected ion flow tube-mass spectrometry (SIFT-MS VoiceUltra 200; Syft Technologies, Anatune, UK). All breath sampling was performed in the morning after a clear fluid diet for a minimum of 6 hours. Exhalation was performed directly into the inlet of the SIFT-MS using a disposable mouthpiece. A baseline breath test was performed for each method followed by consumption of phenylalanine dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C. Data was obtained in concentrations in parts per billion.

### EXAMPLE 4

### Glutamic Acid

Subjects: One healthy subject.

Dose Concentrations

The daily recommended intake levels advised by the Food and Nutrition Board is 30 mg/kg daily for an adult. [1] A single maximum dose of 2.1 g was selected for an average adult of 70 kg.

Breath Sampling

Methods for the detection of short chain fatty acids, aldehydes and phenol-alkanes were established on the selected ion flow tube-mass spectrometry (SIFT-MS VoiceUltra 200; Syft Technologies, Anatune, UK). All breath sampling was performed in the morning after a clear fluid diet for a minimum of 6 hours. Exhalation was performed directly into the inlet of the SIFT-MS using a disposable mouthpiece. A baseline breath test was performed for each method followed by consumption of glutamic acid dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C. Data was obtained in concentrations in parts per billion.

### EXAMPLE 5

### Glycerol Doses

Subjects

Two healthy subjects volunteered for participation and informed written consent was obtained.

Dose Concentrations

Two doses of the substrate were guided by the (i) daily recommended intake levels by the Food and Nutrition Board and (ii) the initial glucose method development study. The daily maximum recommended dose is 276 mg/kg per day for an adult, however, there was no reported harm from higher doses.[1] An average 70 kg individual would be recommended a maximum of 19 g of glycerol. Based on these findings, we selected doses of 50 g, 25 g, 10 g to compare the dose responses against glucose concentration. All findings were compared with a baseline of 0 g.

Breath Sampling

Methods for the detection of short chain fatty acids and aldehydes were established on the selected ion flow tube-mass spectrometry (SIFT-MS VoiceUltra 200; Syft Technologies, Anatune, UK). All breath sampling was performed in the morning and subjects maintained a clear fluid diet for a minimum of 6 hours prior to breath sampling. All subjects exhaled directly into the inlet of the SIFT-MS using a disposable mouthpiece. A baseline breath test was performed for each method followed by consumption of glycerol dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C. Data was obtained in concentrations in parts per billion.

### EXAMPLE 6

### Glycerol Patient Selection

All patients were recruited from St Mary's Hospital from February 2019-December 2019. Patients were recruited from three cohorts; oesophageal cancer (n=6), gastric cancer (n=6) and age-matched healthy controls (n=6). Informed written consent was obtained by all participants. Patients diagnosed with oesophagogastric adenocarcinoma ranged from early disease on the curative pathway to metastatic palliative disease. Age-matched healthy controls included patients with benign upper gastrointestinal disease (reflux, dysmotility) or healthy asymptomatic controls. Demographic and clinical information was collated.

Breath Sampling

Methods for the detection of 4 classes of volatile compounds; short chain fatty acids, alcohols, aldehydes and phenol-alkanes were established on the selected ion flow tube-mass spectrometry (SIFT-MS VoiceUltra 200; Syft Technologies, Anatune, UK). All breath sampling was performed in the morning and patients maintained a clear fluid diet for a minimum of 6 hours prior to breath sampling. All patients exhaled directly into the inlet of the SIFT-MS using a disposable mouthpiece. A baseline breath test was performed for each method followed by consumption of 25 g glycerol dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C.

Statistical Analysis

Data was obtained in concentrations in parts per billion. Univariate Kruskal Wallis analysis was performed across the three groups using SPSS statistical software (v25, Armonk N.Y.; IBM Corp). Mann Whitney U test was performed to identify differences between oesophageal and gastric cancers compared with controls. P value of <0.05 was considered statistically significant.

### EXAMPLE 7

### Combined Amino Acids (Tyrosine, Phenylalanine, Glutamic Acid)

Patient Selection

All patients were recruited from St Mary's Hospital from February 2019-December 2019. Patients were recruited from three cohorts; oesophageal cancer (n=6), gastric cancer (n=1) and age-matched healthy controls (n=6). Informed written consent was obtained by all participants. Patients diagnosed with oesophagogastric adenocarcinoma ranged from early disease on the curative pathway to metastatic palliative disease. Age-matched healthy controls included patients with benign upper gastrointestinal disease (reflux, dysmotility) or healthy asymptomatic controls. Demographic and clinical information was collated.

Breath Sampling

Methods for the detection of 4 classes of volatile compounds; short chain fatty acids, alcohols, aldehydes and phenol-alkanes were established on the selected ion flow tube-mass spectrometry (SIFT-MS VoiceUltra 200; Syft Technologies, Anatune, UK). All breath sampling was performed in the morning and patients maintained a clear fluid diet for a minimum of 6 hours prior to breath sampling. All patients exhaled directly into the inlet of the SIFT-MS using a disposable mouthpiece. A baseline breath test was performed for each method followed by consumption of 2 g tyrosine, 3 g phenylalanine and 2.4 glutamic acid dissolved in 100 mls of warm water, followed by three oral water rinses to decontaminate the oral cavity. Direct sampling was performed for 3 exhaled breath samples over 60 seconds, consecutively with all four methods at five-minute intervals up to 60 minutes (0, 5, 10, 15, 20, 25, 30, 35, 40, 45, 50, 55, 60 mins).

SIFT-MS

SIFT-MS allows real-time quantification and identification of VOCs within the exhaled is breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C.

Statistical Analysis

Data was obtained in concentrations in parts per billion. Mann Whitney U analysis was performed across cancer and non-cancer using SPSS statistical software (v25, Armonk N.Y.; IBM Corp). P value of <0.05 was considered statistically significant.

### EXAMPLE 8

### Combined Glucose and Citric Acid

Patient Selection

All patients were recruited from St Mary's Hospital from February 2019-December 2019. Twelve healthy controls were recruited to form two cohorts to consume; glucose (n=6), and combined glucose and citric acid (n=6). Informed written consent was obtained by all participants. Age-matched healthy controls included patients with benign upper gastrointestinal disease (reflux, dysmotility) or healthy asymptomatic controls.

Breath Sampling

Methods for the detection of 4 classes of volatile compounds; short chain fatty acids, alcohols, aldehydes and phenol-alkanes were established on the selected ion flow tube-mass spectrometry (SIFT-MS VoiceUltra 200; Syft Technologies, Anatune, UK). All breath sampling was performed in the morning and patients maintained a clear fluid diet for a minimum of 6 hours prior to breath sampling. All patients exhaled directly into the inlet of the SIFT-MS using a disposable mouthpiece. A baseline breath test was performed for each method followed by consumption of dissolved in 100 mls of warm water, followed by three oral water rinses to decontaminate the oral cavity. Direct sampling was performed for 3 exhaled breath w samples over 60 seconds, consecutively with all four methods at five-minute intervals up to 30 minutes (0, 5, 10, 15, 20, 25, 30 mins).

SIFT-MS

SIFT-MS allows real-time quantification and identification of VOCs within the exhaled is breath using chemical ionisation. Precursor ions (H3O+, NO+ and O2+) are discharged into a quadrupole mass filter and carried by an inert helium gas along a flow tube. Breath is injected into the flow tube to react with the precursor ions to create product ions which are subsequently separated according to mass-to-charge ratio (m/z). The SIFT-MS was subjected to daily automated validation cycles, to operate within temperatures of 10-30° C.

Statistical Analysis

Data was obtained in concentrations in parts per billion. Mann Whitney U analysis was performed across cancer and non-cancer using SPSS statistical software (v25, Armonk N.Y.; IBM Corp). P value of <0.05 was considered statistically significant.

Results

### EXAMPLE 1

### Glucose Dosing Study

Volatile Organic Compound Analysis

TABLE 1
Median concentrations of short chain fatty acids detected
in the exhaled breath of all subjects at 5-15 minutes.

| Concentration (ppbv) | | | | | Fold change | | | | |
|---|---|---|---|---|---|---|---|---|---|
| Baseline | 10 g | 25 g | 50 g | 75 g | Baseline | 10 g | 25 g | 50 g | 75 g |

Median

| | | | | | | | | | | |
|---|---|---|---|---|---|---|---|---|---|---|
| Acetic acid | 22.1 | 50.2 | 35.5 | 47.3 | 72.6 | 0.8 | 2.3 | 2.0 | 2.0 | 3.0 |
| Butanoic acid | 5.2 | 11.9 | 11.8 | 16.8 | 19.6 | 0.8 | 2.6 | 3.0 | 3.7 | 5.3 |
| Hexanoic acid | 1.1 | 1.2 | 1.0 | 1.0 | 1.8 | 0.8 | 1.6 | 1.4 | 0.9 | 1.2 |
| Pentanoic acid | 5.3 | 5.4 | 5.4 | 6.1 | 10.2 | 0.9 | 1.1 | 1.0 | 1.4 | 1.3 |
| Propanoic acid | 11.5 | 39.6 | 43.4 | 82.3 | 77 | 0.7 | 5.3 | 4.3 | 6.3 | 9.9 |

Average

| | | | | | | | | | | |
|---|---|---|---|---|---|---|---|---|---|---|
| Acetic acid | 28.1 | 57.8 | 36.5 | 43.2 | 68.3 | 1.3 | 2.6 | 1.9 | 2.5 | 2.6 |
| Butanoic acid | 4.7 | 20.7 | 11.8 | 15.4 | 28.7 | 0.9 | 3.5 | 2.8 | 4.4 | 4.8 |
| Hexanoic acid | 1.1 | 1.5 | 1.1 | 1.1 | 1.5 | 0.9 | 1.4 | 1.2 | 0.8 | 1.1 |
| Pentanoic acid | 4.5 | 6.4 | 5.1 | 6.2 | 8.1 | 0.9 | 1.2 | 1.1 | 1.4 | 1.4 |
| Propanoic acid | 12.2 | 96.5 | 46.8 | 82.2 | 120.5 | 1.1 | 7.1 | 4.6 | 7.9 | 7.8 |

Increasing glucose concentrations is positively correlated with increasing concentrations of volatile fatty acids detected within the exhaled breath. Butanoic- and propanoic acid demonstrate a maximal response within 5-15 minutes of glucose consumption with subsequent declining values. Rapid glucose degradation via the glycolytic pathway produces volatile end products detected within exhaled breath. Previous work by the inventors has demonstrated oral water rinsing after glucose consumption eliminates potential VOC response originating from the oral cavity. Butanoic- and pentanoic acid demonstrated a difference of 1-fold increase between 10 g and 50 g of glucose. These compounds were used to guide the recommended glucose dose for preliminary clinical studies involving patients with oesophago-gastric cancer. To obtain a balance between an adequate dose response and a drink acceptable to patients, the inventors selected a dose of 25 g dissolved in 100 ml warm water. The next step of this study will assess the VOC response in patients diagnosed with OG cancer compared to healthy age-matched controls to observe any differences in cellular metabolic activity and VOC response.

TABLE 2
Demographics and clinical information of participants.

| | Oesophageal | Gastric | |
|---|---|---|---|
| | Cancer | Cancer | Controls |
| | (n = 6) | (n = 6) | (n = 6) |
| Age (years) * | 70.5 | 71.5 | 69 |
| Male | 5 | 4 | 3 |
| Ethnicity | | | |
| White | 6 | 5 | 5 |
| Asian | 0 | 0 | 1 |
| Black | 0 | 1 | 0 |
| Metastatic disease | 1 | 1 | — |
| Neoadjuvant therapy | 3 | 5 | — |
| Co-morbidities | | | |
| Diabetes | 1 | 0 | 0 |
| Benign UGI disease | 0 | 0 | 2 |
| Healthy | 0 | 0 | 4 |

* median

TABLE 3
Details of volatile organic compound analysed
by selected ion flow tube mass spectrometry

| Compound | Formula | precursor Ion | Product Ion m/z |
|---|---|---|---|
| Acetone | C₃H₆O | H₃O+ | 59 |
| Short Chain | | | |
| Fatty Acids | | | |
| Acetic acid | CH₃COOH | NO+ | 90 |
| Butanoic acid | C₄H₈O | NO+ | 118 |
| Hexanoic acid | C₆H₁₂O₂ | NO+ | 146 |
| Pentanoic acid | C₅H₁₀O₂ | NO+ | 85 |
| Propanoic acid | CH₃CH₂COOH | NO+ | 104 |
| Aldehydes | | | |
| Acetaldehyde | C₂H₄O | H₃O+ | 45 |
| Decanal | C₁₀H₂₀O | NO+ | 155 |
| Heptanal | C₇H₁₄O | NO+ | 113 |
| Hexanal | C₆H₁₂O | NO+ | 99 |
| Nonanal | C₉H₁₈O | NO+ | 141 |
| Octanal | C₈H₁₆O | NO+ | 127 |
| Pentanal | C₅H₁₀O | NO+ | 85 |
| Butanal | C₄H₈O | NO+ | 71 |
| Propanal | C₃H₆O | NO+ | 57 |
| Phenols | | | |
| 1-hydroxy-4- | C₈H₁₀O | NO+ | 122 |
| ethylbenzene | | | |
| Decane | C₁₀H₂₂ | NO+ | 141 |
| Dodecane | C₁₂H₂₆ | H₃O+ | 189 |
| P-cresol | C₇H₈O | NO+ | 108 |
| Phenol | C₆H₅OH | NO+ | 94 |

Glucose

Volatile Organic Compound Analysis

Short Chain Fatty Acids

TABLE 4
Volatile short chain fatty acids (acetic-, butanoic-, pentanoic-, propanoic acid) increased maximally at 5-10 mins after glucose consumption
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | glucose: | | Baseline | glucose: | | Baseline | glucose | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value* |
| Acetic acid | 38.03 | 31.37 | 0.66 | 19.58 | 23.63 | 1.11 | 16.63 | 39.0 | 2.49 | 5-10 | ↑ | 0.023 |
| Butanoic acid | 5.57 | 9.64 | 2.72 | 4.40 | 12.34 | 1.61 | 4.96 | 34.18 | 10.53 | 5-10 | ↑ | 0.351 |
| Hexanoic acid | 0.64 | 0.52 | 0.87 | 1.00 | 0.65 | 0.63 | 0.76 | 0.63 | 0.87 | 5-10 | — | 0.081 |
| Pentanoic acid | 2.45 | 2.38 | 0.94 | 3.62 | 1.88 | 0.67 | 2.86 | 2.69 | 1.16 | 25-30 | ↑ | <0.001 |
| Propanoic acid | 15.89 | 17.14 | 1.17 | 17.15 | 30.20 | 1.33 | 8.84 | 38.22 | 3.52 | 5-10 | ↑ | 0.243 |

TABLE 5
Mann Whitney U test comparing (i) Control vs. Gastric cancer
groups and (ii) Control vs. Oesophageal cancer groups; p <
0.05 is considered statistically significant (highlighted bold)

| | Control vs Gastric Ca | | Control vs Oesophageal Ca | |
|---|---|---|---|---|
| | | P value | | P value |
| | P value | (Fold | P value | (Fold |
| | (ppbv) | change) | (ppbv) | change) |
| Fatty Acids | | | | |
| Acetic acid | 0.190 | <0.001 | 0.005 | 0.007 |
| Butanoic acid | 0.208 | 0.038 | 0.912 | 1.000 |
| Hexanoic acid | 0.041 | 0.818 | 0.280 | <0.001 |
| Pentanoic acid | <0.001 | <0.001 | 0.018 | 0.011 |
| Propanoic acid | 0.984 | 0.048 | 0.174 | 0.407 |
| Aldehydes | | | | |
| Acetaldehyde | 0.190 | <0.001 | 0.012 | 0.119 |
| Butanal | 0.001 | 0.021 | 0.004 | <0.001 |
| Decanal | <0.001 | 0.779 | 0.001 | 0.646 |
| Heptanal | <0.001 | 0.156 | <0.001 | 0.384 |
| Hexanal | <0.001 | 0.035 | 0.023 | 0.522 |
| Nonanal | 0.001 | 0.031* | 0.006 | 0.008* |
| Octanal | <0.001 | 0.001* | 0.112 | <0.001* |
| Pentanal | <0.001 | <0.001 | 0.004 | 0.818 |
| Propanal | 0.174 | 0.250 | <0.001 | 0.007 |
| Phenol-alkanes | | | | |
| 1-hyroxy-4- | 0.008 | 0.019 | 0.103 | 0.384 |
| ethylbenzene | | | | |
| Decane | <0.001 | <0.001* | <0.001 | <0.001* |
| Dodecane | 0.503 | 0.522 | 0.631 | 0.711 |
| P-cresol | 0.002 | <0.001 | <0.001 | 0.313 |
| Phenol | <0.001* | 0.107 | 0.046* | 0.582 |

Discussion

Three chemical classes of VOCs within exhaled breath have demonstrated a significant difference in patients diagnosed with oesophago-gastric (OG) cancer. A total of 13 compounds from the groups short chain fatty acids (SCFA) (n=4), aldehydes (n=6) and phenols (n=3) have demonstrated increased concentrations after glucose consumption.

Volatile SCFAs demonstrated the largest alteration in breath concentrations; namely butanoic acid and propanoic acid. Optimal concentrations were reached within 10 minutes of consumption suggesting rapid glucose degradation. Glucose, a monosaccharide, enters the glycolytic pathway producing end products of metabolism detected in the breath. Pentanoic acid was detected in higher concentrations at 30 minutes compared to baseline values. These results suggest breath VOCs can be augmented by oral substrates by manipulating the intrinsic metabolic pathways of known VOCs associated with OG cancer. Gastric cancers demonstrate a stronger response than oesophageal cancers in all significant VOCs detected. The gastric cancer group displayed a significant fold change in SCFA (acetic-, butanoic-, pentanoic-, and propanoic acid), with two overlapping significances with the oesophageal cancer group (acetic- and pentanoic acid).

Similarly, aldehydes such as pentanal and propanal followed a similar pattern of response to the SCFA with an optimal increase in concentrations at 5-10 minutes. The remainder of the aldehydes show consistently increased levels in the cancer groups, with 4 of the nine with higher baseline values. Acetaldehyde, butanal, hexanal and pentanal demonstrate a significant increase in fold change from the baseline in gastric is cancer patients. Oesophageal cancer patients show this effect with both butanal and propanal only. On the contrary, both groups demonstrate a significant fold increase in the control groups for nonanal and octanal, which needs to be further explored. These results are consistent with previous work published by the inventors associating volatile butanoic acid, butanal and decanal with OG cancer.[1] The remainder of the aldehydes and the phenol-alkanes (except dodecane) have demonstrated consistently increased concentrations in the cancer groups for the duration of the study. Previous work by the inventors group has implicated phenol as a potential breath biomarker in OG cancer.[2]

Decane, from the phenol family, displays a higher baseline concentration in both cancer groups. A fold increase with the control group after glucose consumption needs to be further explored. A similar pattern of response was observed with P-cresol, but with a fold increase seen only with the gastric cancer group, a new finding. This may be reflective of the transient passage of glucose by the oesophageal tumour compared to pooling of glucose in the stomach.

Currently, NICE guidelines recommend an upper gastrointestinal endoscopy within 2 weeks for patients presenting with ‘red flag’ symptoms suggestive of OG cancer. [3] However, the insidious nature of the disease means the majority present with non-specific symptoms, delaying diagnosis and translating into poor overall survival outcomes. A non-invasive breath test will act as a triage tool to stratify patients with non-specific upper gastrointestinal symptoms. Identification of breath biomarkers for early detection of OG cancer has the potential to offer patients curative treatment and influence overall survival outcomes. This study assessed patients in early and advanced stages of disease.

In clinical practice, exhaled breath could be collected using:
Breath sampling device coupled with thermal desorption tubes to facilitate storage of samples storage and transport.
Direct sampling using mass spectrometry such as SIFT as demonstrated in this study.
Dedicated sensors for those VOC with large response such as acetic-, butanoic-, pentanoic- and propanoic acid.

Key Points

Glucose consumption activates the metabolic pathway associated tumour-microbiome is or increased activity of the tumour cell. This is detected with:
A significant fold increase in SCFA (acetic-, butanoic-, pentanoic- and propanoic acid); more so observed in the gastric cancer than oesophageal cancer group.
A significant increase in aldehydes; acetaldehyde, butanal, hexanal and pentanal in the gastric cancer group. An increase in butanal and propanal are observed with oesophageal cancer.
A new finding of decane and P-cresol in increased concentrations in baseline was observed for both cancer groups. P-cresol fold increase was shown with gastric cancer only.

Exhaled breath samples will be collected at two intervals after glucose ingestion to identify the VOCs at their optimal concentrations; early at 5-10 minutes, and late at 30 minutes.

### EXAMPLE 2

### Tyrosine

TABLE 6
Demographics and clinical information of participants.

| | Oesophageal | Gastric | |
|---|---|---|---|
| | Cancer | Cancer | Controls |
| | (0 = 6) | (n = 6) | (n = 6) |
| Age (years) * | 69.5 | 65 | 66.5 |
| Male | 6 | 5 | 3 |
| Ethnicity | | | |
| White | 5 | 4 | 5 |
| Asian | 1 | 1 | 1 |
| Black | 0 | 1 | 0 |
| Metastatic disease | 0 | 2 | — |
| Neoadjuvant therapy | 3 | 5 | — |
| Co-morbidities | | | |
| Diabetes | 1 | 1 | 0 |
| Benign UGI disease | 0 | 0 | 3 |
| Healthy | 0 | 0 | 3 |

* median

TABLE 7
Details of volatile organic compound analysed
by selected ion flow tube mass spectrometry

| Compound | Formula | precursor Ion | Product Ion m/z |
|---|---|---|---|
| Acetone | C₃H₆O | H₃O+ | 59 |
| Short Chain | | | |
| Fatty Acids | | | |
| Acetic acid | CH₃COOH | NO+ | 90 |
| Butanoic acid | C₄H₈O | NO+ | 118 |
| Hexanoic acid | C₆H₁₂O₂ | NO+ | 146 |
| Pentanoic acid | C₅H₁₀O₂ | NO+ | 85 |
| Propanoic acid | CH₃CH₂COOH | NO+ | 104 |
| Aldehydes | | | |
| Acetaldehyde | C₂H₄O | H₃O+ | 45 |
| Decanal | C₁₀H₂₀O | NO+ | 155 |
| Heptanal | C₇H₁₄O | NO+ | 113 |
| Hexanal | C₆H₁₂O | NO+ | 99 |
| Nonanal | C₉H₁₈O | NO+ | 141 |
| Octanal | C₈H₁₆O | NO+ | 127 |
| Pentanal | C₅H₁₀O | NO+ | 85 |
| Butanal | C₄H₈O | NO+ | 71 |
| Propanal | C₃H₆O | NO+ | 57 |
| Phenols | | | |
| 1-hydroxy-4- | C₈H₁₀O | NO+ | 122 |
| ethylbenzene | | | |
| Decane | C₁₀H₂₂ | NO+ | 141 |
| Dodecane | C₁₂H₂₆ | H₃O+ | 189 |
| P-cresol | C₇H₈O | NO+ | 108 |
| Phenol | C₆H₅OH | NO+ | 94 |

Volatile Organic Compound Analysis

Short Chain Fatty Acids

TABLE 8
Volatile short chain fatty acids demonstrated no significant changes after tyrosine consumption.
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | tyrosine: | | Baseline | tyrosine: | | Baseline | tyrosine | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value* |
| Acetic acid | 37.22 | 34.95 | 0.91 | 25.33 | 13.19 | 0.62 | 22.53 | 24.60 | 1.29 | 25-35 | — | <0.0001 |
| Butanoic acid | 7.08 | 5.85 | 0.98 | 8.60 | 4.56 | 0.69 | 9.77 | 8.61 | 0.86 | 25-35 | — | 0.0068 |
| Hexanoic acid | 1.78 | 1.57 | 0.86 | 1.11 | 0.84 | 0.65 | 1.33 | 0.83 | 0.92 | 25-35 | — | 0.0016 |
| Pentanoic acid | 4.86 | 5.44 | 1.19 | 3.78 | 2.48 | 0.66 | 3.76 | 6.10 | 0.80 | 25-35 | — | <0.0001 |
| Propanoic acid | 22.58 | 16.44 | 0.84 | 17.79 | 8.49 | 0.60 | 31.75 | 25.60 | 0.58 | 25-35 | — | 0.0004 |

*Kruskal Wallis Analysis p < 0.05 considered statistically significant

Aldehydes

TABLE 9
Volatile aldehydes (butanal, decanal, heptanal and hexanal) demonstrated small increases at approximately 30 minutes after tyrosine ingestion.
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | tyrosine: | | Baseline | tyrosine: | | Baseline | tyrosine: | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value * |
| Acetaldehyde | 40.93 | 40.44 | 1.04 | 22.73 | 22.3 | 1.02 | 29.43 | 23.93 | 0.82 | 25-35 | — | 0.0001 |
| Butanal | 5.45 | 5.16 | 0.92 | 4.57 | 4.03 | 1.18 | 5.16 | 4.22 | 0.74 | 25-35 | ↑ | 0.2016 |
| Decanal | 2.04 | 2.11 | 0.89 | 1.08 | 1.27 | 1.11 | 1.14 | 1.08 | 0.83 | 25-35 | ↑ | 0.0001 |
| Heptanal | 1.46 | 2.04 | 1.32 | 0.70 | 0.99 | 1.46 | 0.95 | 1.26 | 1.07 | 25-35 | ↑ | <0.0001 |
| Hexanal | 9.23 | 11.48 | 1.12 | 4.55 | 6.36 | 1.25 | 4.73 | 5.53 | 1.26 | 45 | ↑ | 0.0002 |
| Nonanal | 3.99 | 4.65 | 1.26 | 2.34 | 2.41 | 0.95 | 2.38 | 2.64 | 1.16 | 45 | — | <0.0001 |
| Octanal | 1.38 | 1.70 | 1.11 | 0.91 | 1.04 | 0.94 | 1.08 | 0.93 | 0.79 | 25-35 | — | 0.0002 |
| Pentanal | 2.26 | 3.21 | 0.95 | 1.66 | 1.26 | 0.76 | 2.63 | 2.91 | 1.11 | 25-35 | — | <0.0001 |
| Propanal | 17.98 | 17.12 | 1.01 | 8.52 | 9.05 | 0.94 | 11.70 | 10.51 | 0.85 | 25-35 | — | <0.0001 |

* Kruskal Wallis Analysis p < 0.05 considered statistically significant

Phenols

TABLE 10
Volatile phenols demonstrated a small increase in exhaled breath concentrations
35-45 minutes after tyrosine consumption (excluding 1-hydroxy-4-ethylbenzene).
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | tyrosine: | | Baseline | tyrosine: | | Baseline | tyrosine: | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value * |
| 1-hydroxy-4- | 0.59 | 0.58 | 1.24 | 0.59 | 0.48 | 0.99 | 0.32 | 0.61 | 1.06 | 35-45 | — | 0.0008 |
| ethylbenzene | | | | | | | | | | | | |
| Decane | 5.59 | 8.12 | 1.22 | 3.27 | 3.5 | 1.16 | 3.3 | 4.65 | 1.27 | 35-45 | ↑ | <0.0001 |
| Dodecane | 1.21 | 1.21 | 1.03 | 0.56 | 0.63 | 1.32 | 0.74 | 0.93 | 1.28 | 35-45 | ↑ | 0.0001 |
| P-cresol | 1.24 | 1.59 | 0.90 | 0.92 | 1.18 | 1.12 | 1.72 | 2.70 | 1.10 | 35-45 | ↑ | <0.0001 |
| Phenol | 9.01 | 11.0 | 1.36 | 4.65 | 5.71 | 1.36 | 5.41 | 11.03 | 1.68 | 10 | ↑ | <0.0001 |

* Kruskal Wallis Analysis p < 0.05 considered statistically significant

TABLE 11
Mann Whitney U test comparing (i) Control vs. Gastric cancer
groups and (ii) Control vs. Oesophageal cancer groups; p <
0.05 is considered statistically significant (highlighted bold)

| | Control vs Gastric Ca | | Control vs Oesophageal Ca | |
|---|---|---|---|---|
| | | P value | | P value |
| | P value | (Fold | P value | (Fold |
| | (ppbv) | change) | (ppbv) | change) |
| Fatty Acids | | | | |
| Acetic acid | 0.057 | 0.052 | <0.001* | <0.001* |
| Butanoic acid | 0.395 | 0.001* | 0.017* | <0.001* |
| Hexanoic acid | 0.001* | 0.008* | <0.001* | 0.004* |
| Pentanoic acid | 0.041* | 0.280 | <0.001* | <0.001* |
| Propanoic acid | 0.131 | 0.719 | 0.001* | 0.016* |
| Aldehydes | | | | |
| Acetaldehyde | <0.001* | 0.019* | <0.001* | 0.139 |
| Butanal | 0.021* | 0.003* | 0.430 | 0.190 |
| Decanal | <0.001* | 0.952 | <0.001* | 0.003 |
| Heptanal | <0.001* | <0.001* | <0.001* | 0.741 |
| Hexanal | <0.001* | 0.424 | <0.001* | 0.441 |
| Nonanal | <0.001* | 0.056 | <0.001* | 0.080 |
| Octanal | <0.001* | 0.049* | <0.001* | 0.010* |
| Pentanal | 0.052 | 0.042* | <0.001* | 0.006* |
| Propanal | <0.001* | 0.358 | <0.001* | 0.276 |
| Phenol-alkanes | | | | |
| 1-hyroxy-4- | <0.001* | 0.667 | 0.006* | 0.276 |
| ethylbenzene | | | | |
| Decane | <0.001* | 0.197 | <0.001* | 0.112 |
| Dodecane | <0.001* | 0.023 | <0.001* | 0.230 |
| P-cresol | 0.006 | 0.294 | 0.003* | 0.704 |
| Phenol | 0.005* | 0.535 | <0.001* | 0.139 |

*significant increase in control group.

Discussion

Two chemical classes of volatile compounds (phenols and aldehydes) were detected in slightly higher concentrations 30 minutes after tyrosine consumption. A total of 8 compounds demonstrated small increases in concentrations in the oesophageal cancer group. The underlying biological and mechanistic pathway suggests tyrosine, an aromatic amino acid, is metabolised to phenolic compounds by enzymatic reactions initiated by gastrointestinal bacteria.

Volatile phenol compounds were detected at optimal concentrations at 35-45 minutes after tyrosine consumption, albeit small increases from the baseline values reported. Phenol and decane displayed a similar pattern of increase across groups, whereas P-cresol and dodecane concentrations were detected in slightly higher concentrations in the oesophageal cancer group. Volatile aldehydes, namely butanal, decanal, heptanal and hexanal demonstrated higher concentrations in the oesophageal cancer group is compared with controls (fold change 1.46 vs. 1.32). The overall baseline concentrations of all compounds were notably higher in the control group.

Decanal demonstrated the only significant fold increase in the oesophageal cancer group, supported by previous work by the inventors showing significantly higher baseline values of aldehydes (butanal, decanal) and phenols in OG cancer patients. [1, 2] Lack of corroboration with the inventor's previous findings of baseline concentrations may be attributed to obtaining the results from oesophageal and gastric cancer groups separately and the small patient numbers which needs to be further explored. However, the selected volatile compounds show overall higher responses to tyrosine by fold change in the cancers, albeit not significant.

Short chain fatty acids concentrations from the cancer cohort were not affected by tyrosine.

These results suggest the potential for the augmentation of breath VOCs with oral metabolic substrates acting via the shikimate pathway. In the next phase of the study, the inventors intend to use phenylalanine, a precursor to tyrosine, in addition to tyrosine, as a combination drink. Without wishing to be bound to any particular theory, the inventors propose measuring breath VOC concentrations between 30-45 minutes after ingestion to detect any potential changes with the addition an amino acid.

Key Points:
Decanal, from the aldehyde family, demonstrates a significant fold increase after tyrosine consumption in the oesophageal cancer group.
Aldehyde and phenol compounds show a slightly higher fold change from baseline values, albeit not significant.
The significantly higher baseline aldehyde and phenol values in the control groups needs to be further explored.
Volatile phenol compounds were detected at optimal concentrations at 35-45 minutes after tyrosine consumption.

### EXAMPLE 3

### Phenylalanine

Results and Discussion

Phenylalanine is an essential amino acid, a known precursor to other amino acids such as tyrosine. Metabolism via the shikimate pathway is expected to produce volatile phenol compounds. Three compounds from the phenol family (dodecane, decane and phenol) demonstrated increasing concentrations after phenylalanine consumption (FIGS. 18 to 20). Dodecane and decane shows a maximal increase at 10-15 minutes after consumption (3.2 and 1.8-fold increase, respectively). Phenol showed a 2.7-fold increase at 60 minutes. Decanal and dodecane show an elevated response to phenylalanine in comparison to tyrosine which has no noticeable effect (FIG. 21). Phenol produced similar end results, whereas decane show slightly higher values after phenylalanine ingestion.

### EXAMPLE 4

### Glutamic Acid

Results and Discussion

Three compounds from the aldehyde and phenol family demonstrated an increase in VOC concentrations after glutamic acid consumption (FIGS. 22 to 25). Propanal showed the elevated concentrations maximally at 5 minutes with a fold change of 3.5. Both dodecane and phenol showed up to 2-fold increase at 20 and 45 minutes respectively. Glutamic acid is a non-essential amino acid metabolised via the shikimate pathway producing volatile compounds from the phenol family. Glutamic acid is involved in a transamination process during degradation. The resultant keto-acid is used as a key intermediate in the citric acid cycle for further cellular metabolism. This may account for the slight increase observed with butanoic acid within 5 minutes of glutamic acid consumption.

Without wishing to be bound to any particular theory, the inventors believe that in combination with other amino acids tested, phenylalanine and tyrosine, an augmented VOC response may be produced, in particular with consistent compounds already identified across the groups: dodecane, phenol.

### EXAMPLE 5

### Glycerol Doses

Results

Subjects

Two subjects were recruited with an average age of 32 years; 1 female vs 1 male. No significant co-morbidities were noted.

Volatile Organic Compound Analysis

TABLE 12
Concentrations of short chain fatty acids detected in
the exhaled breath of each subject at 45-55 minutes.

| Concentration (ppbv) | | | Fold change | | |
|---|---|---|---|---|---|
| Baseline | 25 g | 50 g | Baseline | 25 g | 50 g |

Subject 1

| | | | | | | |
|---|---|---|---|---|---|---|
| Acetic acid | 37.4 | 19.3 | 53.2 | 1.8 | 1.2 | 2.7 |
| Butanoic acid | 4.0 | 4.0 | 10.2 | 0.9 | 1.4 | 2.5 |
| Hexanoic acid | 0.6 | 0.4 | 1.3 | 0.7 | 0.3 | 0.7 |
| Pentanoic acid | 2.0 | 0.7 | 4.9 | 1.2 | 0.4 | 1.9 |
| Propanoic acid | 12.6 | 13.4 | 38.1 | 1.8 | 1.1 | 4.1 |

Subject 2

| | | | | | | |
|---|---|---|---|---|---|---|
| Acetic acid | 25.4 | 37.9 | 22.2 | 0.9 | 0.6 | 1.1 |
| Butanoic acid | 6.6 | 13.0 | 7.2 | 0.9 | 1.3 | 1.5 |
| Hexanoic acid | 1.7 | 2.2 | 2.7 | 0.9 | 0.6 | 0.9 |
| Pentanoic acid | 7.3 | 9.0 | 4.4 | 1.1 | 1.1 | 1.3 |
| Propanoic acid | 14.7 | — | 16.0 | 0.7 | — | 2.0 |

Discussion

Increasing glycerol concentrations translates to increased production of volatile fatty acids detected within the exhaled breath. Volatile fatty acid concentrations from 25 g glycerol are comparable to the baseline values. Butanoic acid concentrations are elevated after 30 minutes of glycerol ingestion, with maximal concentrations detected at 45-55 minutes. Glycerol is a polyol compound found in lipids and is metabolised via the glycolytic pathway by (i) direct entry into the pathway or (ii) be converted to glucose by gluconeogenesis. The glucose study demonstrated maximal fatty acid detection at 5-10 minutes after glucose consumption, and therefore it is expected responses after glycerol ingestion are delayed as it may require further enzymatic reactions before entry into the cycle. The inventors propose using a dose of 50 g to elicit a fatty acid VOC response within the breath of patients diagnosed with OG cancer compared to healthy age-matched controls.

### EXAMPLE 6

### Glycerol

Results

Patients

Eighteen patients were recruited (n=6 within each group; oesophageal cancer, gastric cancer, healthy controls). All cancers included were histologically confirmed as adenocarcinomas.

TABLE 13
Demographics and clinical information of participants.

| | Oesophageal | Gastric | |
|---|---|---|---|
| | Cancer | Cancer | Controls |
| | (n = 6) | (n = 6) | (n = 6) |
| Age (years) * | 72.5 | 60 | 64.5 |
| Male | 6 | 4 | 1 |
| Ethnicity | | | |
| White | 6 | 2 | 4 |
| Asian | 0 | 1 | 1 |
| Black | 0 | 0 | 0 |
| Arabic | 0 | 3 | 1 |
| Metastatic disease | 1 | 2 | — |
| Neoadjuvant therapy | 6 | 2 | — |
| Co-morbidities | | | |
| Diabetes | | | 0 |
| Benign UGI disease | | | 2 |
| Healthy | | | 4 |

* median

Volatile Organic Compound Analysis

Short Chain Fatty Acids

TABLE 14
Volatile short chain fatty acids (acetic-, butanoic-, propanoic acid) increased maximally at 60 mins after glycerol consumption.
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | glycerol: | | Baseline | glycerol: | | Baseline | glycerol | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value* |
| Acetic acid | 35.68 | 26.90 | 0.62 | 54.77 | 101.35 | 1.50 | 20.99 | 33.60 | 1.09 | 60 | ↑ | 0.007 |
| Butanoic acid | 7.46 | 8.59 | 1.12 | 10.55 | 18.93 | 2.02 | 4.99 | 8.67 | 1.43 | 60 | ↑ | <0.001 |
| Hexanoic acid | 1.56 | 1.13 | 0.52 | 2.04 | 1.43 | 0.49 | 1.95 | 0.98 | 0.70 | 60 | — | 0.002 |
| Pentanoic acid | 3.53 | 3.75 | 0.83 | 5.49 | 7.87 | 1.05 | 2.86 | 2.49 | 0.86 | 60 | — | <0.001 |
| Propanoic acid | 24.70 | 26.95 | 0.72 | 45.95 | 107.93 | 1.75 | 10.19 | 19.07 | 1.46 | 60 | ↑ | <0.001 |

*Kruskai Wallis Analysis p < 0.05 considered statistically significant

Aldehydes

TABLE 15
Volatile aldehydes were maximally increased for the oesophageal cancer group between 40-55 minutes (hexanal, octanal, pentanal, propanal).
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | glycerol: | | Baseline | glycerol: | | Baseline | glycerol | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value * |
| Acetaldehyde | 25.47 | 32.05 | 0.88 | 51.00 | 19.30 | 0.80 | 23.72 | 17.85 | 0.83 | 60 | — | <0.001 |
| Butanal | 3.39 | 4.44 | 0.87 | 9.02 | 6.49 | 1.00 | 2.78 | 2.21 | 1.05 | 60 | — | <0.001 |
| Decanal | 1.31 | 1.18 | 0.97 | 2.53 | 1.46 | 0.77 | 1.17 | 0.95 | 1.05 | 60 | — | <0.001 |
| Heptanal | 2.28 | 1.33 | 0.58 | 2.48 | 1.40 | 0.76 | 1.60 | 1.20 | 0.65 | 60 | — | <0.001 |
| Hexanal | 6.28 | 6.93 | 1.17 | 7.77 | 11.56 | 1.72 | 4.42 | 5.52 | 1.09 | 40 | ↑ | <0.001 |
| Nonanal | 3.07 | 2.97 | 0.98 | 3.62 | 3.71 | 1.09 | 3.36 | 2.93 | 1.09 | 60 | — | <0.001 |
| Octanal | 1.49 | 1.35 | 0.95 | 1.91 | 1.68 | 1.58 | 1.25 | 1.39 | 0.87 | 40 | ↑ | <0.001 |
| Pentanal | 2.62 | 1.39 | 0.63 | 2.37 | 3.71 | 1.27 | 1.30 | 1.63 | 1.46 | 55 | ↑ | <0.001 |
| Propanal | 15.77 | 12.51 | 0.77 | 16.93 | 17.83 | 1.71 | 7.38 | 5.67 | 0.98 | 55 | ↑ | <0.001 |

* Kruskal Wallis Analysis p < 0.05 considered statistically significant

Phenols

TABLE 16
Volatile phenols demonstrated no alterations in exhaled breath concentrations between the three patient groups.
Median

| | Control | | | Oesophageal Cancer | | | Gastric Cancer | | | | | |
|---|---|---|---|---|---|---|---|---|---|---|---|---|
| | | Post- | | | Post- | | | Post- | | | | |
| | Baseline | glycerol: | | Baseline | glycerol: | | Baseline | glycerol | | | | |
| | Concen- | Concen- | | Concen- | Concen- | | Concen- | Concen- | | Time | | |
| | tration | tration | Fold | tration | tration | Fold | tration | tration | Fold | point | Increase/ | P |
| | (ppbv) | (ppbv) | change | (ppbv) | (ppbv) | change | (ppbv) | (ppbv) | change | (mins) | decrease | value * |
| 1-hydroxy-4- | 0.72 | 1.09 | 1.47 | 1.14 | 0.88 | 0.99 | 0.56 | 0.56 | 1.47 | 60 | — | — |
| ethylbenzene | | | | | | | | | | | | |
| Decane | 5.33 | 4.61 | 0.92 | 6.54 | 7.33 | 1.13 | 5.83 | 4.89 | 0.81 | 60 | — | — |
| Dodecane | 1.03 | 0.75 | 0.86 | 1.02 | 1.30 | 1.09 | 0.84 | 1.05 | 0.95 | 60 | — | — |
| P-cresol | 1.71 | 1.46 | 0.71 | 2.23 | 2.41 | 1.04 | 0.96 | 0.83 | 0.98 | 60 | — | — |
| Phenol | 4.21 | 2.64 | 1.43 | 4.73 | 7.71 | 1.36 | 4.18 | 3.68 | 0.98 | 60 | — | — |

* Kruskal Wallis Analysis p < 0.05 considered statistically significant

TABLE 17
Mann Whitney U test comparing (i) Control vs Gastric cancer
groups and (ii) Control vs Oesophageal cancer groups;
p < 0.05 is considered statistically significant

| | Control vs Gastric Ca | | Control vs Oesophageal Ca | |
|---|---|---|---|---|
| | | P value | | P value |
| | P value | (Fold | P value | (Fold |
| | (ppbv) | change) | (ppbv) | change) |
| Fatty Acids | | | | |
| Acetic acid | 0.103 | <0.001 | 0.005 | 0.070 |
| Butanoic acid | 0.258 | 0.012 | <0.001 | 0.008 |
| Hexanoic acid | 0.147 | 0.711 | <0.001 | 0.435 |
| Pentanoic acid | <0.001 | 0.779 | <0.001 | 0.029 |
| Propanoic acid | 0.242 | <0.001 | <0.001 | 0.005 |
| Aldehydes | | | | |
| Acetaldehyde | <0.001 | 0.112 | 0.056 | 0.009 |
| Butanal | <0.001 | <0.001 | <0.001 | 0.053 |
| Decanal | 0.048 | <0.001 | <0.001 | 0.803 |
| Heptanal | <0.001 | 0.271 | 0.503 | 0.459 |
| Hexanal | 0.005 | 0.833 | <0.001 | 0.582 |
| Nonanal | 0.107 | 0.060 | <0.001 | 0.322 |
| Octanal | 0.043 | 0.008 | <0.001 | <0.001 |
| Pentanal | 0.001 | <0.001 | <0.001 | <0.001 |
| Propanal | <0.001 | <0.001 | <0.001 | <0.001 |
| Phenol-alkanes | | | | |
| 1-hyroxy-4- | 0.008 | 0.503 | 0.009 | 0.569 |
| ethylbenzene | | | | |
| Decane | 1.000 | 0.944 | <0.001 | 0.056 |
| Dodecane | 0.056 | 0.003 | <0.001 | <0.001 |
| P-cresol | 0.003 | 0.002 | <0.001 | 0.002 |
| Phenol | 0.222 | 0.764 | 0.014 | 0.682 |

Discussion

Three chemical classes of VOCs within exhaled breath have demonstrated a significant increase in patients diagnosed with oesophago-gastric (OG) cancer. Glycerol is a polyol compound found in lipids and is metabolised via the glycolytic pathway by (i) direct entry into the pathway or (ii) converted to glucose by gluconeogenesis. The glucose study demonstrated maximal fatty acid detection at 5-10 minutes after glucose consumption, and therefore it is expected elevated responses after glycerol ingestion may be delayed further as enzymatic reactions may be required before entry into the cycle.

In keeping with the hypothesis, we observed elevation in short chain fatty acids (SCFA) and aldehydes levels between 45-60 minutes after glycerol consumption, as illustrated in FIGS. 30A to 30E. SCFA, namely acetic-, butanoic- and propanoic acids, showed a large increase in the oesophageal cancer group (1.5, 2.02, 1.75-fold increase, respectively) compared to the gastric cancer group (1.09, 1.43, 1.46-fold increase, respectively). Optimal concentrations were reached at 60 minutes, with a gradual increase observed after 45 minutes.

Similarly, select aldehydes were found to increase largely in the oesophageal cancer group (FIGS. 31A to 31I). Hexanal and propanal showed the highest increases with 1.7-fold increases between 40-55 minutes. Octanal increased with 1.58-fold change and pentanal with 1.27-fold change. The gastric cancer group showed a change with only pentanal at 1.46-fold increase at 55 minutes. The remainder of the aldehydes were unaffected.

A number of the volatile phenols tested demonstrated no significant alterations in exhaled breath concentrations between the three patient groups after glycerol consumption (FIGS. 32A to 32E).

Glycerol consumption has uniquely increased target VOCs in the oesophageal cancer group. Potentially this may be due to the higher viscosity of the fluid coating the oesophagus allowing more than a transient passage. Increased contact time between the substrate and the tumour may explain the higher VOC levels produced.

Key Points:

Glycerol consumption activates the glycolytic metabolic pathway associated tumour-microbiome or increased activity of the tumour cell. This is detected by:
A significant fold increase in SCFA (acetic-, butanoic-, and propanoic acid); more so observed in the oesophageal cancer group than the gastric cancer group.
A significant increase in aldehydes; hexanal, octanal, pentanal and propanal in the oesophageal cancer. An increase in pentanal was observed with gastric cancer.

### EXAMPLE 7

### Combined Amino Acids (Tyrosine, Phenylalanine, Glutamic Acid)

Results

Patients

Thirteen patients were recruited (oesophageal cancer n=6, gastric cancer n=1, healthy controls n=6). All cancers included were histologically confirmed as adenocarcinomas.

TABLE 18
Demographics and clinical information of participants.

| | Oesophagogastric | |
|---|---|---|
| | Cancer | Controls |
| | (n = 7) | (n = 6) |
| Age (years) * | 65 | 70 |
| Male | 6 | 3 |
| Ethnicity | | |
| White | 6 | 6 |
| Asian | 1 | 0 |
| Black | 0 | 0 |
| Arabic | 0 | 0 |
| Metastatic disease | 4 | — |
| Neoadjuvant therapy | 5 | — |
| Co-morbidities | | |
| Diabetes | 2 | 0 |
| Benign UGI disease | — | 2 |
| Healthy | — | 0 |

* median

Volatile Organic Compound Analysis

Short Chain Fatty Acids

TABLE 19
Volatile short chain fatty acids were not altered after amino acid consumption.
Median

| | Control | | | Oesophagogastric Cancer | | | | | | |
|---|---|---|---|---|---|---|---|---|---|---|
| | | Post-amino | | | Post-amino | | | | | |
| | Baseline | acids: | | Baseline | acids: | | Time | | | |
| | Concentration | Concentration | Fold | Concentration | Concentration | Fold | point | Increase/ | P value* | P value |
| | (ppbv) | (ppbv) | change | (ppbv) | (ppbv) | change | (mins) | decrease | ppbv | Fold change |
| Acetic acid | 25.65 | 24.74 | 0.67 | 24.57 | 29.15 | 0.99 | 30 | — | — | — |
| Butanoic acid | 5.10 | 3.74 | 0.88 | 8.13 | 7.76 | 0.15 | 30 | — | — | — |
| Hexanoic acid | 0.71 | 0.65 | 0.94 | 1.10 | 0.89 | 0.80 | 30 | — | — | — |
| Pentanoic acid | 2.08 | 1.74 | 1.01 | 2.24 | 1.53 | 0.84 | 30 | — | — | — |
| Propanoic acid | 13.77 | 9.95 | 1.10 | 11.70 | 11.46 | 0.87 | 30 | — | — | — |

*Mann Whitney U analysis p < 0.05 considered statistically significant

Aldehydes

TABLE 20
Volatile decanal demonstrated an increase in the oesophagogastric cancer
group at 30 minutes after consumption of the combined amino acid drink.

| | Control | | | Oesophagogastric Cancer | | | | | | |
|---|---|---|---|---|---|---|---|---|---|---|
| | | Post-amino | | | Post-amino | | | | | |
| | Baseline | acids: | | Baseline | acids: | | Time | | | |
| | Concentration | Concentration | Fold | Concentration | Concentration | Fold | point | Increase/ | P value* | P value* |
| | (ppbv) | (ppbv) | change | (ppbv) | (ppbv) | change | (mins) | decrease | ppbv | Fold change |
| Acetaldehyde | 16.95 | 16.08 | 0.93 | 30.05 | 19.80 | 0.68 | 30 | — | — | — |
| Butanal | 2.39 | 1.73 | 1.01 | 2.48 | 2.42 | 1.08 | 30 | — | — | — |
| Decanal | 0.64 | 0.59 | 1.05 | 0.69 | 0.83 | 1.41 | 30 | ↑ | <0.001 | <0.001 |
| Heptanal | 1.23 | 0.75 | 1.10 | 1.20 | 0.94 | 0.81 | 30 | — | — | — |
| Hexanal | 2.73 | 3.16 | 1.31 | 2.52 | 3.42 | 1.24 | 30 | — | — | — |
| Nonanal | 1.68 | 1.88 | 0.98 | 2.21 | 2.55 | 1.16 | 30 | — | — | — |
| Octanal | 0.82 | 0.56 | 0.90 | 0.88 | 0.59 | 0.78 | 30 | — | — | — |
| Pentanal | 1.06 | 0.72 | 1.00 | 0.98 | 1.14 | 0.97 | 30 | — | — | — |
| Propanal | 12.15 | 9.54 | 0.95 | 8.55 | 8.78 | 1.06 | 30 | — | — | — |

*Mann Whitney U analysis p < 0.05 considered statistically significant

Phenol-Alkanes

TABLE 21
Volatile phenols demonstrated an increase of p-cresol in exhaled breath concentrations between the cancer
and non-cancer groups. The increase in phenol concentrations were comparable between both groups.

| | Control | | | Oesophagogastric Cancer | | | | | | |
|---|---|---|---|---|---|---|---|---|---|---|
| | | Post-amino | | | Post-amino | | | | | |
| | Baseline | acids: | | Baseline | acids: | | Time | | | |
| | Concentration | Concentration | Fold | Concentration | Concentration | Fold | point | Increase/ | P value * | P value * |
| | (ppbv) | (ppbv) | change | (ppbv) | (ppbv) | change | (mins) | decrease | ppbv | Fold change |
| 1-hydroxy-4- | 0.64 | 0.44 | 0.98 | 0.65 | 0.45 | 0.87 | 30 | — | 0.289 | 0.003 |
| ethylbenzene | | | | | | | | | | |
| Decane | 3.59 | 4.45 | 1.25 | 3.87 | 4.78 | 1.44 | 30 | ↑ | 0.303 | 0.066 |
| Dodecane | 0.52 | 0.92 | 1.38 | 0.76 | 0.77 | 1.09 | 30 | — | 0.147 | 0.089 |
| P-cresol | 1.26 | 1.22 | 0.84 | 0.93 | 1.25 | 1.37 | 40 | ↑ | 0.073 | <0.001 |
| Phenol | 3.15 | 4.82 | 1.79 | 1.59 | 2.27 | 1.83 | 30 | ↑ | <0.001 | 0.051 |

* Mann Whitney U analysis p < 0.05 considered statistically significant

Discussion

Two chemical classes, aldehydes and phenol-alkanes, demonstrated an increase in volatile organic compound levels after the consumption of three combined amino acids, as illustrated in FIGS. 33 and 34A to 34E. In contrast, only decanal was slightly elevated in the oesophageal cancer group when tyrosine alone was administered.

Decanal, an aldehyde, demonstrated a more significant increase in detected levels with this combination amino acid drink (FIG. 33). A fold increase of 1.41 was observed in the cancer group (baseline=0.69 ppbv, 30 minutes=0.83 ppbv) compared to a fold increase of 1.05 in the control group. The maximum concentrations occurred at 30 minutes after consuming the nutrient drink.

Phenol-alkanes are the primary target of this nutrient group. Pathways have been detailed describing the metabolism of tyrosine by tyrosine phenol lyase to produce phenols. More recently, Saito et al have delineated a pathway involving metabolism by the enzyme tyrosine lyase to produce p-cresol. This metabolic pathway has been proven within bacteria, not the human cells [4]. P-cresol was significantly increased at 40 minutes after consumption of the amino acid drink from baseline levels of 0.93 ppbv to 1.25 ppbv translating to a 1.37-fold increase. No change was observed in the control group. Phenol showed a global increase across both cancer (1.79-fold increase) and non-cancer groups (1.83-fold increase), with no significant differences between the two. Decane also increased at 30 minutes with a 1.44-fold increase in the cancer group.

There were no significant alterations in the remainder of the aldehydes and short chain fatty acid groups. Further work needs to be done to explain the increase in decanal.

Key Points:

Consumption of combined amino acids potentially activates a metabolic pathway associated with bacteria. This is detected with:
A significant fold increase in decanal (aldehyde).
A global increase in phenol (enzyme tyrosine phenol lyase), with no differences between cancer and non-cancer groups.
A new finding of a significant increase in P-cresol, potentially produced by the enzymatic metabolism using tyrosine lyase.

### EXAMPLE 8

### Combined Glucose and Citric Acid

TABLE 22
Demographics and clinical information of participants.

| | Control group 1 | Control group 2 |
|---|---|---|
| | (glucose only) | (glucose + citric acid) |
| | (n = 6) | (n = 6) |
| Age (years) * | 69 | 67.5 |
| Male | 3 | 2 |
| Ethnicity | | |
| White | 5 | 5 |
| Asian | 1 | 0 |
| Black | 0 | 1 |
| Arabic | 0 | 0 |
| Co-morbidities | | |
| Diabetes | 0 | 0 |
| Benign UGI disease | 2 | 3 |
| Healthy | 4 | 3 |

* median

Volatile Organic Compound Analysis

Short Chain Fatty Acids

TABLE 23
Volatile short chain fatty acids (butanoic- and propanoic acid) had increased concentrations detected in control group 2 (glucose + citric acid).
Median

| | Control 1 (glucose only) | | | Control 2 (glucose + citric acid) | | | | | | |
|---|---|---|---|---|---|---|---|---|---|---|
| | Baseline | Post-drink: | | Baseline | Post-drink: | | Time | | | |
| | Concentration | Concentration | Fold | Concentration | Concentration | Fold | point | Increase/ | P value* | P value |
| | (ppbv) | (ppbv) | change | (ppbv) | (ppbv) | change | (mins) | decrease | ppbv | Fold change |
| Acetic acid | 38.03 | 31.37 | 0.66 | 19.02 | 17.93 | 0.94 | 5 | — | <0.001 | 0.032 |
| Butanoic acid | 5.57 | 9.64 | 2.72 | 2.88 | 14.90 | 5.36 | 5 | ↑ | 0.960 | 0.093 |
| Hexanoic acid | 0.64 | 0.52 | 0.87 | 1.06 | 0.63 | 0.62 | 5 | — | 0.008 | 0.246 |
| Pentanoic acid | 2.45 | 2.38 | 0.73 | 3.11 | 2.65 | 0.99 | 5 | — | 0.003 | 0.142 |
| Propanoic acid | 15.89 | 17.14 | 1.17 | 8.19 | 15.33 | 1.96 | 5-10 | ↑ | 0.024 | 0.093 |

*Mann Whitney U analysis p < 0.05 considered statistically significant

Aldehydes

TABLE 24
Volatile decanal demonstrated an increase in the oesophagogastric cancer group at 30 minutes after consumption of the combined amino acid drink.
Median

| | Control 1 (glucose only) | | | Control 2 (glucose + citric acid) | | | | | | |
|---|---|---|---|---|---|---|---|---|---|---|
| | Baseline | Post-drink: | | Baseline | Post-drink: | | Time | | | |
| | Concentration | Concentration | Fold | Concentration | Concentration | Fold | point | Increase/ | P value* | P value* |
| | (ppbv) | (ppbv) | change | (ppbv) | (ppbv) | change | (mins) | decrease | ppbv | Fold change |
| Acetaldehyde | 16.02 | 15.25 | 1.04 | 19.72 | 21.09 | 0.94 | 5 | — | — | — |
| Butanal | 2.46 | 2.21 | 1.03 | 2.82 | 2.37 | 0.87 | 5 | — | — | — |
| Decanal | 0.66 | 0.59 | 0.97 | 0.87 | 0.77 | 1.13 | 5 | — | — | — |
| Heptanal | 0.60 | 0.53 | 0.86 | 1.80 | 1.22 | 0.77 | 5 | — | — | — |
| Hexanal | 2.71 | 3.09 | 1.06 | 4.04 | 4.86 | 1.07 | 5 | — | — | — |
| Nonanal | 1.07 | 1.56 | 1.23 | 2.30 | 2.49 | 1.08 | 5 | — | — | — |
| Octanal | 0.43 | 0.58 | 1.40 | 1.43 | 1.12 | 0.81 | 5 | — | — | — |
| Pentanal | 1.17 | 1.05 | 1.00 | 1.32 | 1.43 | 0.89 | 5 | — | — | — |
| Propanal | 9.67 | 9.29 | 1.06 | 6.96 | 9.41 | 1.29 | 10-15 | ↑ | 0.001 | 0.912 |

*Mann Whitney U analysis p < 0.05 considered statistically significant

Discussion

Two chemical classes, short chain fatty acids (SCFA) and aldehydes, demonstrated an increase in volatile organic compound levels after the consumption of glucose and citric acid combined, as can be seen in FIGS. 35A to 35E and 36. The results of Example 1 demonstrate a significant increase in these groups within 5-10 minutes of glucose consumption alone. The hypothesis states that glucose is metabolised via the glycolytic pathway which occurs in human cells and bacterial cells. The glycolytic pathway feeds into the citric acid cycle and therefore, the objective was to assess a further increase in VOCs with the addition of citric acid.

Butanoic acid demonstrated the largest increase from 2.72-fold with glucose alone to 5.36-fold with the addition of citric acid (FIG. 35B). The maximal concentrations were achieved within 5-10 of consumption of the drink. Propanoic acid also demonstrated an increase of 1.96-fold with the addition of citric acid (FIG. 35E). No remarkable changes were observed with the remainder of the SCFA group.

Propanal appeared to be the only aldehyde to show an increase of 1.29-fold in the citric acid group within 10-15 minutes, albeit a small change (FIG. 36). No significant alterations were observed in the remainder of the aldehydes group.

Clear alterations in VOCs have been shown in two control groups, with citric acid as the differentiating factor. We hypothesis these nutrients may feed into the glycolytic and citric acid intrinsic metabolic pathways.

Key Points:

Consumption of combined glucose and citric acid activates known metabolic pathways associated with cell metabolism. This is detected by:
A significant fold increase in volatile short chain fatty acids (butanoic- and propanoic acid) within 5-10 minutes of consumption of the nutrient drink.
A significant fold increase in propanal within 10-15 minutes.

The next steps will involve recruiting patients with oesophago-gastric cancer to assess breath VOC alterations in response to additional nutritional substrates.

### REFERENCES

1. Markar, S. R., et al., Assessment of a Noninvasive Exhaled Breath Test for the Diagnosis of Oesophagogastric Cancer. JAMA Oncol, 2018. 4(7): p. 970-976.

2. Kumar K, H. J., Abbassi-Ghadi N, Mackenzie H A, Veselkov K A, Hoare J M, Lovat L B, Spanel P, Smith D and Hanna G B, Mass Spectrometric Analysis of Exhaled Breath for the Identification of Volatile Organic Compound Biomarkers in Esophageal and Gastric Adenocarcinoma. Annals of Surgery, 2015. 262(6): p. 981-990.

3. Excellence, N. I. o. C., Gastrointestinal tract (upper) cancers—recognition and referral. 2016.

4. Saito Y, Sato T, Nomoto K, Tsuji H. Identification of phenol- and p-cresol-producing intestinal bacteria by using media supplemented with tyrosine and its metabolites. FEMS Microbiol Ecol. 2018. 94(9)

1. A method for diagnosing a subject suffering from cancer, or a pre-disposition thereto, or for providing a prognosis of the subject's condition, the method comprising:
(i) detecting, in a bodily sample from a test subject, the concentration of a signature compound resulting from the metabolism of at least one sugar and/or at least one amino acid or a precursor thereof and/or at least one polyol present in a composition previously administered to the subject, wherein the sugar is present in the composition at a concentration of more than 20,000 mg/100 ml, the amino acid or a precursor thereof is present in the composition at a concentration of at least 500 mg/ml and the polyol is present in the composition at a concentration of more than 25,000 mg/100 ml; and
(ii) comparing this concentration with a reference for the concentration of the signature compound in an individual who does not suffer from cancer, wherein an increase or a decrease in the concentration of the signature compound compared to the reference, suggests that the subject is suffering from cancer, or has a pre-disposition thereto, or provides a negative prognosis of the subject's condition.

2. The method according to claim 1, wherein the detection step (i) comprises detecting a signature compound up to 30 minutes, up to 25 minutes, up to 20 minutes, up to 15 minutes, up to 10 minutes or up to 5 minutes from administration of the composition comprising at least one sugar and/or an amino acid or a precursor thereof and/or at least one polyol.

3. The method according to claim 1 or claim 2, wherein detection step (i) comprises detecting a signature compound at between 30 and 60, or between 30 and 55 minutes, or between 30 and 50 minutes, or between 30 and 45 minutes, or between 30 and 40 minutes, or between 35 and 60 minutes, or between 35 and 55 minutes, or between 35 and 50 minutes, or between 35 and 45 minutes, or between 35 and 40 30 minutes from administration of the composition comprising at least one sugar and/or an amino acid or a precursor thereof and/or at least one polyol.

4. The method according to any preceding claim, wherein an increase in the concentration of the signature compound compared to the reference suggests that the subject is suffering from cancer, or has a pre-disposition thereto, or provides a negative prognosis of the subject's condition.

5. The method according to claim 4, wherein the increase in the concentration of the signature compound is at least a 10%, 20%, 30%, 40%, 50%, 100%, 200%, 300%, 400%, 500%, 600%, 700%, 800%, 900% or 1000% increase in the concentration of signature compound when compared to the reference.

6. The method according to any preceding claim, wherein the sugar is present in the composition previously administered to the subject at a concentration of at least 20,500 mg/100 ml.

7. The method according to any preceding claim, wherein the sugar is glucose, sorbitol, mannose or lactose.

8. The method according to any preceding claim, wherein the sugar is glucose and is present in the composition previously administered to the subject at a concentration of at least 25,000 mg/100 ml and the signature compound is detected up to 10 minutes from administration of the composition comprising glucose.

9. The method according to any preceding claim, wherein the composition administered to the subject comprises citric acid in combination with the sugar, wherein the citric acid is present in the composition at a concentration of at least 1,000 mg/100 ml, optionally wherein the sugar is glucose.

10. The method according to any preceding claim, wherein the amino acid is selected from a group consisting of: tyrosine, glutamic acid, glutamate, phenylalanine, tryptophan, proline and histidine, optionally wherein the composition comprises tyrosine, phenylalanine and glutamic acid.

11. The method according to claim 10, wherein the amino acid is tyrosine and is present in the composition previously administered to the subject at a concentration of at least 2,000 mg/100 ml, optionally wherein the signature compound is detected between 35 and 45 minutes from administration of the composition comprising tyrosine.

12. The method according to any preceding claim, wherein the amino acid precursor is phenylalanine, optionally present at a concentration of at least 3000 mg/100 ml.

13. The method according to any preceding claim, wherein the polyol is glycerol.

14. The method according to any preceding claim, wherein the polyol is present in the composition at a concentration of more than 30,000 mg/100 ml.

15. The method according to any preceding claim, wherein the cancer is oesophago-gastric junction cancer, gastric cancer, oesophageal cancer, oesophageal squamous-cell carcinoma (ESCC) or oesophageal adenocarcinoma (EAC).

16. The method according to any preceding claim, wherein the cancer is gastric cancer, oesophageal cancer or a metastasised cancer.

17. The method according to any preceding claim, wherein the signature compound is a short chain fatty acid, aldehyde, alcohol or any combination thereof.

18. The method according to claim 17, wherein the signature compound is a C1-C3 aldehyde, a C1-C3 alcohol, a C2-C10 alkane wherein a first carbon atom is substituted with the ═O group and a second carbon atom is substituted with an —OH group, a C1-C20 alkane, a C4-C10 alcohol, a C1-C6 carboxylic acid, a C4-C20 aldehyde, phenol optionally substituted with a C1-C6 alkyl group, a C2 aldehyde, a C3 aldehyde, a C8 aldehyde, a C9 aldehyde, a C10 aldehyde, a C11 aldehyde, an analogue or derivative of any aforementioned species, or any combination thereof.

19. The method according to either claim 17 or claim 18, wherein the signature compound is selected from a group consisting of: acetic acid, butanoic acid, hexanoic acid, pentanoic acid, propanoic acid, acetaldehyde, decanal, heptanal, hexanal, nonanal, octanal, pentanal, butanal, propanal, 1-hydroxy-4-ethylbenzene, decane, dodecane, P-cresol, and phenol, or any combination thereof.

20. The method according to any one of claims 17 to 19, wherein the substrate is a sugar, preferably glucose, and the signature compound is acetic acid, butanoic acid, pentanoic acid, propanoic acid, hexanoic acid, acetaldehyde, propanal, butanal, hexanal, pentanal, decanal, 1-hydoxytheylbenzene and/or P-cresol.

21. The method according to any one of claims 17 to 19, wherein the substrate is an amino acid or precursor thereof, and the signature compound is butanal, decanal, heptanal, hexanal, phenol, decane, P-cresol, 1-hydoxytheylbenzene and/or dodecane.

22. The method according to claim 21, wherein the amino acid or precursor thereof is tyrosine, and the signature compound is decanal and/or dodecane.

23. The method according to any one of claims 17 to 19, wherein the substrate is a polyol, preferably glycerol, and the signature compound is butanoic acid, acetic acid, hexanoic acid, pentanoic acid, propanoic acid, butanal, hexanal, pentanal, and/or propanal.

24. A method for detecting a signature compound in a test subject, the method comprising:
(i) providing the subject with a composition comprising at least one substrate according to any preceding claim into a signature compound; and
(ii) detecting the concentration of the signature compound in a bodily sample from the subject.

25. The method according to claim 24, wherein the signature compound is as defined in any one of claims 17 to 23.

26. A composition comprising at least one sugar and/or at least one amino acid or a precursor thereof and/or at least one polyol present suitable for metabolism into a signature compound, wherein the sugar is present in the composition at a concentration of more than 20,000 mg/100 ml and the amino acid is present in the composition at a concentration of at least 500 mg/ml and the polyol is present in the composition at a concentration of more than 25,000 mg/100 ml, for use in a method of diagnosis or prognosis.

27. A composition comprising at least one sugar and/or at least one amino acid or a precursor thereof and/or at least one polyol present suitable for metabolism into a signature compound, wherein the sugar is present in the composition at a concentration of more than 20,000 mg/100 ml and the amino acid is present in the composition at a concentration of at least 500 mg/ml and the polyol is present in the composition at a concentration of more than 25,000 mg/100 ml for use in a method of diagnosing or prognosing cancer, optionally wherein the cancer is oesophago-gastric junction cancer, gastric cancer, oesophageal cancer, oesophageal squamous-cell carcinoma (ESCC) or oesophageal adenocarcinoma (EAC).

28. A composition comprising at least one substrate according to any one of claims 1 to 14, for use in the method according to any one of claims 1 to 23.

29. A kit for diagnosing a subject suffering from cancer, or a pre-disposition thereto, or for providing a prognosis of the subject's condition, the kit comprising:
(a) a composition comprising at least one substrate as defined in any one of claims 1 to 14;
(b) means for determining the concentration of a signature compound in a sample from a test subject; and
(c) a reference for the concentration of the signature compound in a sample from an individual who does not suffer from cancer,
wherein the kit is used to identify an increase or a decrease in the concentration of the signature compound in the bodily sample from the test subject, compared to the reference, thereby suggesting that the subject suffers from cancer, or has a pre-disposition thereto, or provides a negative prognosis of the subject's condition.

30. The kit according to claim 29, wherein the signature compound is as defined in any one of claims 17 to 23.

31. A method for determining the efficacy of treating a subject suffering from cancer with a therapeutic agent or a specialised diet or chemotherapy or chemoradiotherapy, the method comprising:
(i) providing the subject with a composition comprising at least one substrate according to any one of claims 1 to 14; and
(ii) analysing the concentration of the signature compound resulting from metabolism of the at least one substrate in a bodily sample from a test subject, and comparing this concentration with a reference for the concentration of the signature compound in an individual who does not suffer from cancer,
wherein an increase or a decrease in the concentration of the signature compound in the bodily sample from the test subject compared to the reference suggests that the treatment regime with the therapeutic agent or the specialised diet or chemotherapy or chemoradiotherapy is effective or ineffective.

32. The method according to claim 31, wherein the signature compound is as defined in any one of claims 17 to 23.

33. The method according to either claim 31 or claim 32, wherein the cancer is oesophago-gastric junction cancer, gastric cancer, oesophageal cancer, oesophageal squamous-cell carcinoma (ESCC) or oesophageal adenocarcinoma (EAC).
